(19) United States
(12) Patent Application Publication
(10) Pub. No.: US 2011/0224170 A1
(43) Pub. Date: Sep. 15, 2011

(54) Pyranopyranone derivatives as antimicrobial agents

(75) Inventors: Barbara Wagner, Lorrach (DE); Janina Purschwitz, Freiburg (DE); Karin Bieler, Lorrach (DE)

(73) Assignee: BASF SE, Ludwigshafen (DE)

(21) Appl. No.: 13/028,576

(22) Filed: Feb. 16, 2011

Related U.S. Application Data
(60) Provisional application No. 61/309,449, filed on Mar. 2, 2010.

(51) Int. Cl. A61K 31/366; A61K 31/695; A01N 43/90; A01N 55/10; C07D 493/04; A61P 31/00; A01P 1/00

(52) U.S. Cl. 514/63; 514/456; 514/455; 514/453; 549/290

(57) ABSTRACT

Disclosed is the use of pyranopyranone derivatives of formula

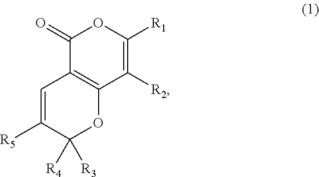

wherein
R₁, R₂, R₃, R₄ and R₅ independently of one another are hydrogen; C₁-C₃₀alkyl, C₂-C₃₀alkenyl, or C₃-C₁₂cycloalkyl, which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₇-C₂₅aralkyl, or —CO—R₆; —SiR₈R₉R₁₀; or
R₄ and R₅ together form a five or six membered ring; or
R₃ and R₄ together form a five or six membered ring;
D is —CO—; —COO—; —S—; —SO—; —SO₂—; —O—; —NR₇—; —SiR₈R₉—; —POR₁₀—; —CR₁₂═CR₁₃—; or —C≡C—; and
E is —OR₆; —SR₆; —NR₁₄R₁₅; —NR₁₄COR₁₅; —COR₆; —COOR₆; —CONR₁₄R₁₅; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈ perfluoroalkyl; C₁-C₁₈alkoxy, which is optionally substituted by E and/or interrupted by D; wherein
R₆ is H; C₆-C₁₈aryl which is optionally substituted by OH, C₁-C₃₀alkyl or C₁-C₃₀alkoxy; or C₁-C₃₀alkyl which is optionally interrupted by —O—;
R₇ is H; C₆-C₁₈aryl which is optionally substituted by OH, C₁-C₃₀alkyl or C₁-C₃₀alkoxy; or C₁-C₃₀alkyl which is optionally interrupted by —O—;
R₈, R₉ and R₁₀ independently of each other are hydrogen; C₁-C₃₀alkyl; C₆-C₁₈aryl which is optionally substituted by C₁-C₃₀alkyl; and
R₁₁ is hydrogen; C₆-C₁₈aryl, which is optionally substituted by OH, C₁-C₃₀alkyl or C₁-C₃₀alkoxy; or C₁-C₃₀alkyl, which is optionally interrupted by —O—;
R₁₂, R₁₃, R₁₄ and R₁₅ independently of each other are hydrogen; C₆-C₁₈aryl which is optionally substituted by OH, C₁-C₃₀alkyl or C₁-C₃₀alkoxy; C₁-C₃₀alkyl, which is optionally interrupted by —O—; or
R₁₄ and R₁₅ together form a five or six membered ring;
   as antimicrobial and/or preserving agents.

This application claims the benefit of U.S. Provisional Application No. 61/309,449 filed Mar. 2, 2010 herein incorporated entirely by reference.

### DESCRIPTION

The present invention relates to the use of specific pyranopyranone derivatives for the antimicrobial treatment of surfaces, as antimicrobial active substances against gram-positive and gram-negative bacteria, yeasts and fungi and also for the preservation of cosmetics, household products, textiles and plastics and for use as disinfectants.

Therefore, the present invention relates to the use of pyranopyranone derivatives of formula

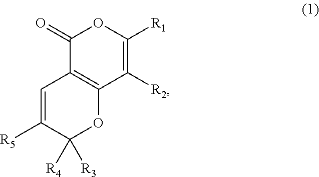

wherein
R₁, R₂, R₃, R₄ and R₅ independently of one another are hydrogen; C₁-C₃₀alkyl, C₂-C₃₀alkenyl, or C₃-C₁₂cycloalkyl, which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₁-C₂₅aralkyl, or —CO—R₆; —SiR₈R₉R₁₀; or
R₄ and R₅, together with the carbon atom they are attached to, form a five or six membered ring; or
R₃ and R₄, together with the carbon atoms they are attached to, form a five or six membered ring;
D is —CO—; —COO—; —S—; —SO—; —SO₂—; —O—; —NR₇; —SiR₈R₉—; —POR₁₀—; —CR₁₂═CR₁₃—; or —C≡C—; and
E is —OR₆; —SR₆; —NR₁₄R₁₅; —NR₁₄COR₁₅; —COR₆; —COOR₆; —CONR₁₄R₁₅; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈ perfluoroalkyl; C₁-C₁₈alkoxy, which is optionally substituted by E and/or interrupted by D; wherein
R₆ is H; O₆—C₁₈aryl which is optionally substituted by OH, C₁-C₃₀alkyl or C₁-C₃₀alkoxy; or C₁-C₃₀alkyl which is optionally interrupted by —O—;
R₆ is H; C₆-C₁₈aryl which is optionally substituted by OH, C₁-C₃₀alkyl or C₁-C₃₀alkoxy; or C₁-C₃₀alkyl which is optionally interrupted by —O—;
R₈, R₉ and R₁₀ independently of each other are hydrogen; C₁-C₃₀alkyl; C₆-C₁₈aryl which is optionally substituted by C₁-C₃₀alkyl; and
R₁₁ is hydrogen; C₆-C₁₈aryl, which is optionally substituted by OH, C₁-C₃₀alkyl or C₁-C₃₀alkoxy; or C₁-C₃₀alkyl, which is optionally interrupted by —O—;
R₁₂, R₁₃, R₁₄ and R₁₅ independently of each other are hydrogen; C₆-C₁₈aryl which is optionally substituted by OH, C₁-C₃₀alkyl or C₁-C₃₀alkoxy; C₁-C₃₀alkyl, which is optionally interrupted by —O—; or
R₁₄ and R₁₅ together form a five or six membered ring;
as antimicrobial and/or preserving agents (collectively named “antimicrobial compound” in the following).

C₁-C₃₀alkyl are straight chain or branched radicals like methyl, ethyl, n-propyl, iso-propyl, n-butyl, sek-butyl, tert-butyl, amyl, isoamyl or tert-amyl, hexyl, 2-ethylhexyl, heptyl, octyl, isooctyl, nonyl, decyl, undecyl, dodecyl, tetradecyl, undecyl, eicosyl, tetracosyl, pentacosyl, heptacosyl, octacosyl or triacontyl.

C₁-C₃₀alkoxy are straight chain or branched radicals like methoxy, ethoxy, n-propoxy, isopropoxy, n-butyloxy, sek-butyloxy, tert-butyloxy, amyloxy, isoamyloxy, tert-amyloxy, hexyloxy, 2-ethylhexyloxy, heptyloxy, octyloxy, isooctyloxy, nonyloxy, decyloxy, undecyloxy, dodecyloxy, tetradecyloxy, undecyloxy eicosyloxy tetracosyloxy, pentacosyloxy, heptacosyloxy, octacosyloxy or triacontyloxy.

C₂-C₃₀alkenyl are straight chain or branched radicals like allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, iso-dodecenyl, n-dodec-2-enyl, n-octadec-4-enyl, eicosenyl, tetracosenyl, pentacoensyl, heptacosenyl, octacosenyl or triacontenyl.

C₃-C₁₂cycloalkyl is for example cyclopropyl, cyclobutyl, cyclopentyl, cycloheptyl, cyclooctyl, cyclononyl, cyclodecyl, cycloundecyl, cyclododecyl and preferably cyclohexyl.

Each alkyl or cycloalkyl can be saturated or unsaturated.

Each alkyl, cycloalkyl or alkoxy can preferably be substituted by one or more E and/or interrupted by one or more D.

Each aryl can be preferably substituted by G; adjacent residues G may be interconnected to form, for example, C₃-C₄alkylene, or a residue —O—CH₂—O—.

Each heteroaryl can be preferably substituted by G, C₂-C₁₈alkenyl, C₂-C₁₈alkynyl, C₇-C₂₅aralkyl, CN, or —CO—R₆.

Hetero-C₃-C₁₂aryl is preferably pyridinyl, pyrimidinyl, triazinyl, pyrrolyl, furanyl, thio-phenyl or chinolinyl.

C₆-C₂₀aryl is for example naphthyl and preferably phenyl or biphenyl.

Preference is given to compounds wherein R₁ is C₁-C₃₀alkyl and even more preferred are compounds wherein R₁ is methyl.

Further preference is given to compounds of formula (I), wherein
R₁ is C₁-C₆alkyl;
R₂ is hydrogen;
R₃, R₄ and R₅ independently of one another are hydrogen; C₁-C₃₀alkyl, C₂-C₃₀alkenyl, or C₃-C₁₂cycloalkyl, which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₇-C₂₅aralkyl, or —CO—R₆; or
R₄ and R₅ together form a five or six membered ring;

D is —CO—; —COO—; —O—; —NR₇—; —CR₁₂═CR₁₃—; or —C≡C—; and
E is —OR₆; —NR₁₄R₁₅; —NR₁₄COR₁₅; —COR₆; —COOR₆; —CONR₁₄R₁₅; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈ perfluoroalkyl; C₁-C₁₈alkoxy, which is optionally substituted by E and/or interrupted by D; wherein
R₆, R₇, R₁₁, R₁₂, R₁₃, R₁₄ and R₁₅ are defined as in formula (1).

Further preference is given to compounds of formula (1), wherein
R₁ is C₁-C₆alkyl;
R₂ is hydrogen;
R₃, R₄ and R₅ independently of one another are hydrogen; C₁-C₃₀alkyl, C₂-C₃₀alkenyl, or C₃-C₁₂cycloalkyl, which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₇-C₂₅aralkyl, or —CO—R₆; or
R₄ and R₅ together form a five or six membered ring;
D is —CO—; —COO—; —O—; —NR₇—; —CR₁₂═CR₁₃—; or —C≡C—; and
E is —OR₆; —NR₁₄R₁₅; —NR₁₄COR₁₅; —COR₆; —COOR₆; —CONR₁₄R₁₅; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈ perfluoroalkyl; C₁-C₁₈alkoxy, which is optionally substituted by E and/or interrupted by D; wherein
R₆, R₇, R₁₁, R₁₂, R₁₃, R₁₄ and R₁₅ are defined as in formula (I).

More preferred are compounds of formula (1), wherein
R₁ is C₁-C₄alkyl;
R₂ is hydrogen;
R₃, R₄ and R₅ independently of one another are hydrogen; C₁-C₃₀alkyl, or C₂-C₃₀alkenyl, which may be substituted by one or more E and/or interrupted by one or more D; C₆-C₂₀aryl, which may be substituted by one or more G; C₄-C₂₀heteroaryl, which may be substituted by one or more G; or
R₄ and R₅ together form a five or six membered ring;
D is —CO—; —COO—; —O—; —NR₇—; —CR₁₂═CR₁₃—; or —C≡C—; and
E is —OR₆; —NR₁₄R₁₅; —NR₁₄COR₁₅; —COR₆; —COOR₆; or —CONR₁₄R₁₅;
G is E; C₁-C₁₈alkyl, which is optionally interrupted by D; C₁-C₁₈alkoxy, which is optionally substituted by E and/or interrupted by D; wherein
R₆, R₇, R₁₂, R₁₃, R₁₄ and R₁₅ are defined as in formula (I).

Most preferred are compounds of formula (1), wherein
R₁ is methyl;
R₂ is hydrogen;
R₃, R₄ and R₅ independently of one another are hydrogen; C₁-C₃₀alkyl, or C₂-C₃₀alkenyl, or

R₃ and R₄ together form a five or six membered ring, which may be interrupted by —O—; or
R₄ and R₅ together form a five or six membered ring.
   R₃ and R₄, or R₄ and R₅, together with the carbon atom(s) they are attached to may form a five or six membered ring as noted above; thus, R₃ and R₄, or R₄ and R₅, together may, for example, be C2-C8alkylene, C2-C8alkenylene, C2-C8oxaalkylene, C2-C8oxaalkenylene, C4-C8cycloalkylene, C4-C8cycloalkenylene. In consequence, the rings formed may be carbocyclic or heterocyclic, and mono- or bicyclic; examples are C5-C6cycloalkyl, C5-C6cycloalkenyl, C6-C7bicycloalkyl, C6-C7bicycloalkenyl, C4-C5oxacycloalkyl, C4-C5oxacycloalkenyl, each of which being unsubstituted or substituted by G, such as in the examples for R3/R4 and R4/R5 listed below.

Even more preferred are compounds of formula (1), wherein

R₁ is methyl

R₂ is hydrogen

R₃ is hydrogen; C₁-C₁₂alkyl; C₂-C₄alkenyl; or a radical of formula

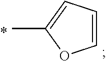

R₄ is hydrogen; C₂-C₄alkenyl;

R₅ is hydrogen; C₁-C₄alkyl; phenyl; —SiR₈, R₉R₁₈; or a radical of formula

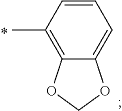

or

R₃ and R₄ together form the rings

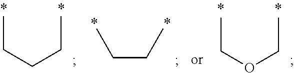

or

R₄ and R₅ together form the rings

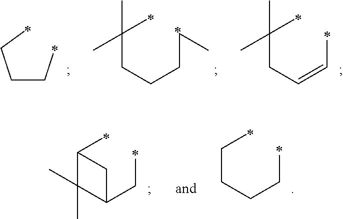

Examples of pyranopyranone compounds according to the present invention are listed in Table 1 below:

TABLE 1
Representatives of pyranopyranones according to the present invention

| | |
|---|---|
| P-1 | 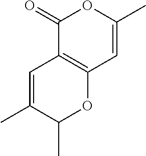 |
| P-2 | 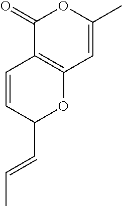 |
| P-3 | 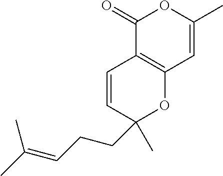 |
| P-4 | 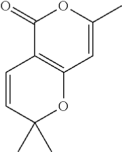 |
| P-5 | 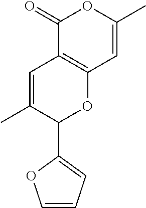 |
| P-6 | 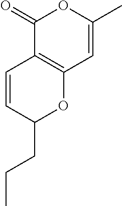 |
| P-7 | 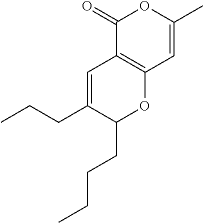 |
| P-8 | 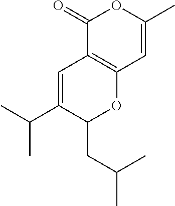 |
| P-9 | 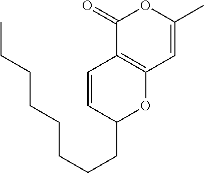 |
| P-10 | 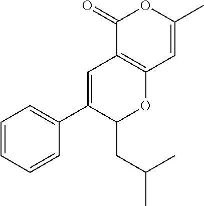 |
| P-11 | 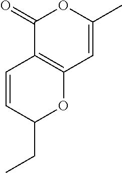 |
| P-12 | 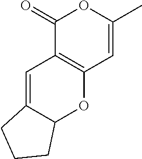 |
| P-13 | 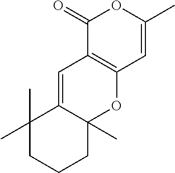 |
| P-14 | 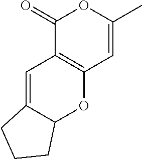 |
| P-15 | 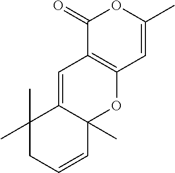 |
| P-16 | 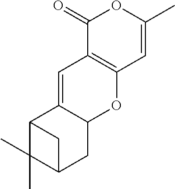 |
| P-17 | 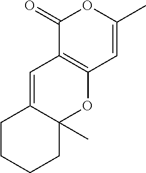 |
| P-18 | 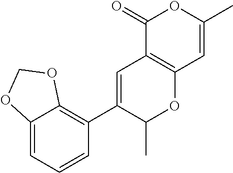 |
| P-19 | 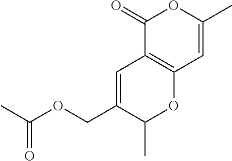 |
| P-20 | 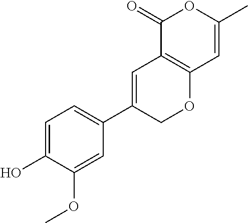 |
| P-21 | 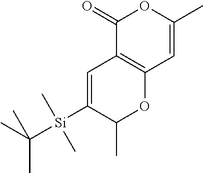 |
| P-22 | 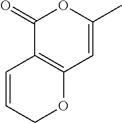 |
| P-23 | 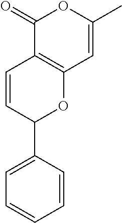 |
| P-24 | 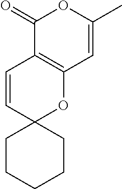 |
| P-25 | 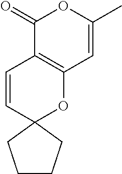 |
| P-26 | 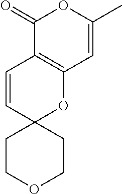 |

Selected compounds are

(P-1); (P-2); (P-3); (P-4); (P-6); and (P-7).

Most preferred compounds are (P-3, (P-4) and (P-6).

One of the pyranopyranones defined above is novel and is also a subject of the present invention.

The novel compound correspond to the formula

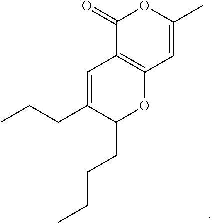

The pyranopyranones corresponding to formula (1) can be prepared according to standard procedures known per se, generally by reacting a pyranone compound corresponding to formula (1a) with an aldehyde corresponding to formula (1b) to form the pyranopyranone compound corresponding to formula (1) wherein the substituents are as defined above.

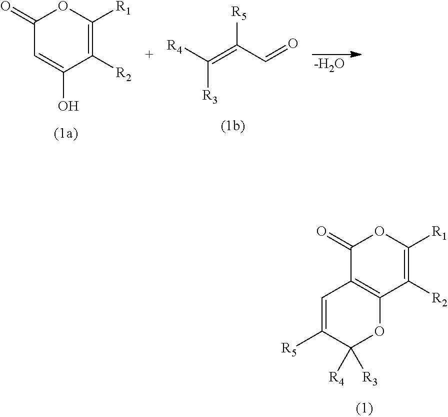

This condensation reaction is generally performed in suitable solvents such as water, ethyl acetate, acetone, acetonitrile, dioxane, chloroform, tetrahydrofurane, dimethyl-formamide, diethylether or tert-butylmethylether, in the presence of an organic or inorganic base, preferably calcium sulfate or potassium carbonate.

Such procedures are described for example in the journals Bioorganic & Medicinal Chemistry Letter, volume 17 (2007) on the pages 978 to 982 or in QSAR & Combinatorial Science, 23(10), (2004), on the pages 895-898; or in Carbohydrate Research, 340(7), (2005), on the pages 1287-1300; or in Organic Letters, volume 8(2), (2006), on the pages 191-193; or in Journal of Organic Chemistry, volume 68(5), (2003), on the pages 1729-1735; or in Journal of Organic Chemistry, volume 64(3), (1999) on the pages 690-691; or in Journal of Organic Chemistry, volume 62(20), (1997) on the pages 6888-6896.

The compounds of the present invention also exhibit pronounced antimicrobial action, for example against pathogenic gram-positive and gram-negative bacteria and against bacteria of the skin and oral flora, and also against yeasts and molds. They are accordingly suitable for disinfection, deodorization, and for general and antimicrobial treatment of the skin and mucosa and of integumentary appendages (hair), for example, for the disinfection of hands and wounds. Compounds of the invention thus may be used for the preparation of a medicament for skin, mucosal, oral, or wound disinfection. The invention thus includes a method, or a use, for the preparation of a medicament for protecting skin, mucosa and/or integumentary appendages against the action of microbes, including protecting the scalp from dandruff, which method comprises incorporation of an effective amount of a pyranopyranone derivative of formula (1) into said medicament.

Of special interest is the non-therapeutic use of the present compounds as antimicrobial and/or preserving agents.

They are accordingly suitable as antimicrobial active substances and preservatives in personal care preparations, for example shampoos, bath additives, hair care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, wet wipes, oils or powders.

For example, the antimicrobial compounds of the invention are effective as anti-dandruff agents in shampoos or other products used in hair treatment such as oils, sprays, hair gels.

Moreover they can be used as agent against dermatophytes for use in products against seborrheic dermatitis, psoriasis and athletes foot in formulation types such as creams, lotions, gels, powders, oils, tonics, sprays, wet wipes etc. And as anti-acne agents in facial care products such as creams, lotions, gels, tonics, powders etc.

The invention accordingly relates also to a personal care preparation comprising at least one antimicrobial compound of the invention and cosmetically tolerable carriers or adjuvants.

The personal care preparation according to the invention, generally contains from 0.01 to 15% by weight, for example, from 0.1 to 10% by weight, based on the total weight of the inventive composition, of the antimicrobial compounds of the invention, and cosmetically tolerable adjuvants and/or carrier. Likewise, the medicament generally contains from 0.01 to 15% by weight, for example, from 0.1 to 10% by weight, based on the total weight of the inventive composition, of the antimicrobial compounds of the invention, and pharmaceutically tolerable adjuvants and/or carrier. Pharmaceutically tolerable adjuvants and/or carriers are usually selected from suitable ingredients, or classes of ingredients, listed below for personal care preparations of the invention.

Depending upon the form of the personal care preparation, it comprises, in addition to the antimicrobial compounds of the invention, further constituents, for example sequestering agents, colorings, perfume oils, thickening or solidifying agents (consistency regulators), emulsifiers, emollients, UV-absorbers, skin protective agents, anti-oxidants, additives that improve the mechanical properties, such as dicarboxylic acids and/or aluminium, zinc, calcium or magnesium salts of C₁₄-C₂₂fatty acids, and, optionally, preservatives.

The personal care preparation according to the invention may be in the form of a water-in-oil or oil-in-water emulsion, an alcoholic or alcohol-containing formulation, a vesicular dispersion of an ionic or non-ionic ampiphilic lipid, a gel, a solid stick or an aerosol formulation.

As a water-in-oil or oil-in-water emulsion, the cosmetically tolerable adjuvant contains preferably from 5 to 50% of an oil phase, from 5 to 20% of an emulsifier and from 30 to 90% water. The oil phase may comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in various fields. There come into consideration, for example, the following preparations:
skin-care preparations, e.g. skin-washing and cleansing preparations in the form of tablet-form or liquid soaps, synthetic detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eye shadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams or oils, sun-blocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, after-shaves or aftershave lotions;
fragrance preparations, e.g. fragrances (eau de Cologne, eau de toilette, eau de parfum, parfum de toilette, perfume), perfume oils or perfume creams;
dental care, denture-care and mouth-care preparations, e.g. toothpastes, gel tooth-pastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or chamomile.

The invention relates also to oral compositions containing from 0.01 to 15% by weight, based on the total weight of the composition, of the antimicrobial compound, and orally tolerable adjuvants.

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name OLAFLUOR.

The anti-microbial compounds of this invention are also suitable for treating, especially preserving, textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose.

The antimicrobial compounds of this invention are suitable also for treating, especially imparting antimicrobial properties to or preserving, plastics, e.g. polyethylene, polypropylene, polyurethane, polyester, polyamide, polycarbonate, latex etc. Fields of use therefore are, for example, floor coverings, plastics coatings, plastics containers and packaging materials; kitchen and bathroom utensils (e.g. brushes, shower curtains, sponges, bathmats), latex, filter materials (air and water filters), plastics articles used in the field of medicine, e.g. dressing materials, syringes, catheters etc., so-called “medical devices”, gloves and mattresses.

The antimicrobial compounds of this invention are suitable also for treating, especially imparting antimicrobial properties to or preserving industrial formulations such as coatings, lubricants etc.

Paper, for example papers used for hygiene purposes, may also be provided with anti-microbial properties using the present anti-microbial compounds.

It is also possible for nonwovens, e.g. nappies/diapers, sanitary towels, panty liners, and cloths for hygiene and household uses, to be provided with antimicrobial properties in accordance with the invention.

The antimicrobial compounds of this invention are also used in washing and cleaning formulations, e.g. in liquid or powder washing agents or softeners.

The antimicrobial compounds of this invention can also be used in household and general-purpose cleaners for cleaning and disinfecting hard surfaces.

In addition to preserving cosmetic and household products, the preservation of technical products, the provision of technical products with antimicrobial properties and use as a biocide in technical processes are also possible, for example in paper treatment, especially in paper treatment liquors, printing thickeners of starch or cellulose derivatives, surface-coatings and paints.

The antimicrobial compounds of the invention are also suitable for the antimicrobial treatment of wood and for the antimicrobial treatment of leather, the preserving of leather and the provision of leather with antimicrobial properties.

The compounds according to the invention are also suitable for the protection of cosmetic products, pharmaceutical products and household products from microbial dam-age.

The compounds claimed in the present invention can be combined with biocides used as antimicrobial actives in cosmetic and pharmaceutical products and household products and/or preservatives used in cosmetic and pharmaceutical products or household products in order to improve the antimicrobial activity of the cosmetic product, the pharmaceutical product or household product.

In addition, the compounds are capable of penetrating biofilms on living and non-living surfaces, of preventing the adhesion of bacteria to surfaces and any further build-up of the biofilm, of detaching such biofilm and/or inhibiting the further growth of the biofilm-forming micro-organisms in the biological matrix, or of killing such micro-organisms.

Biofilms are understood, very generally, to be aggregations of living and dead micro-organisms, especially bacteria, that adhere to living and non-living surfaces, together with their metabolites in the form of extracellular polymeric substances (EPS matrix), e.g. polysaccharides. The activity of antimicrobial substances that normally exhibit a pronounced growth-inhibiting or lethal action with respect to planktonic cells may be greatly reduced with respect to microorganisms that are organized in biofilms, for example because of inadequate penetration of the active substance into the biological matrix.

In the present invention, this may relate to biofilms on human tooth surfaces and oral mucosa, which play a crucial role in the onset of degenerative diseases in the oral cavity, e.g. caries or periodontitis, as a result of the biofilm-forming micro-organisms or their metabolites.

Action against bio-films in the present invention also relates to biofilms on non-human surfaces. The antimicrobial compounds of this invention are useful in coatings or films in protecting surfaces from bio-fouling. Such surfaces include surfaces in contact with marine environments (including fresh water, brackish water and salt water environments), for example, the hulls of ships, surfaces of docks or the inside of pipes in circulating or pass-through water systems. Other surfaces are susceptible to similar bio-fouling, for example walls exposed to rain water, walls of showers, roofs, gutters, pool areas, saunas, floors and walls exposed to damp environs such as basements or garages and even the housing of tools and outdoor furniture.

The antimicrobial compounds of this invention are also useful in preventing bio-fouling, or eliminating or controlling microbe accumulation on the surfaces either by incorporating the antimicrobial compounds into the article or surface of the article in question or by applying the antimicrobial to these surfaces as part of a coating or film.

When applied as a part of a film or coating, the antimicrobial compounds of this invention are part of a composition which also comprises a binder.

The binder may be any polymer or oligomer compatible with the present antimicrobials. The binder may be in the form of a polymer or oligomer prior to preparation of the anti-fouling composition, or may form by polymerization during or after preparation, including after application to the substrate. In certain applications, such as certain coating applications, it will be desirable to crosslink the oligomer or polymer of the anti fouling composition after application.

The term binder as used in the present invention also includes materials such as glycols, oils, waxes and surfactants commercially used in the care of wood, plastic, glass and other surfaces. Examples include water proofing materials for wood, vinyl protectants, protective waxes and the like.

The composition may be a coating or a film. When the composition is a thermoplastic film which is applied to a surface, for example, by the use of an adhesive or by melt applications including calendaring and co-extrusion, the binder is the thermoplastic polymer matrix used to prepare the film.

When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer.

Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g. from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention.

Alkyd resins, polyesters, polyurethanes, epoxy resins, silicone containing polymers, polyacrylates, polyacrylamides, fluorinated polymers and polymers of vinyl acetate, vinyl alcohol and vinyl amine are non-limiting examples of common coating binders useful in the present invention. Other coating binders, of course, are part of the present invention.

Coatings are frequently crosslinked with, for example, melamine resins, urea resins, isocyanates, isocyanurates, polyisocyanates, epoxy resins, anhydrides, poly acids and amines, with or without accelerators.

The compositions of present invention are for example a coating applied to a surface which is exposed to conditions favorable for bioaccumulation. The presence of the antimicrobial compounds of this invention in said coating will prevent the adherence of organisms to the surface.

The anti-microbial compounds of the present invention may be part of a complete coating or paint formulation, such as a marine gel-coat, shellac, varnish, lacquer or paint, or the anti fouling composition may comprise only a polymer of the instant invention and binder, or a polymer of the instant invention, binder and a carrier substance. It is anticipated that other additives encountered in such coating formulations or applications will find optional use in the present applications as well.

The coating may be solvent borne or aqueous. Aqueous coatings are typically considered more environmentally friendly.

The coating is, for example, aqueous dispersion of a polymer of the instant invention and a binder or a water based coating or paint. For example, the coating comprises an aqueous dispersion of a polymer of the instant invention and an acrylic, methacrylic or acrylamide polymers or co-polymers or a polky[-alkoxyalkanoate-co-3-hydroxyalkenoate] polyester.

The coating may be applied to a surface which has already been coated, such as a protective coating, a clear coat or a protective wax applied over a previously coated article.

Coating systems include marine coatings, wood coatings, other coatings for metals and coatings over plastics and ceramics. Exemplary of marine coatings are gel coats comprising an unsaturated polyester, a styrene and a catalyst.

The coating is, for example a house paint, or other decorative or protective paint. It may be a paint or other coating that is applied to cement, concrete or other masonry article. The coating may be a water proofer as for a basement or foundation.

The coating composition is applied to a surface by any conventional means including spin coating, dip coating, spray coating, draw down, or by brush, roller or other applicator. A drying or curing period will typically be needed.

Coating or film thickness will vary depending on application and will become apparent to one skilled in the art after limited testing.

The composition may be in the form of a protective laminate film.

Such a film typically comprises thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyl resins, epoxy resins, unsaturated polyesters, unsaturated polyamides, polyimides, fluorinated polymers, silicon containing and carbamate polymers. The polymers may also be blends and copolymers of the preceding chemistries.

When the anti-fouling composition is a preformed film it is applied to the surface by, for example, the use of an adhesive, or co-extruded onto the surface. It may also be mechanically affixed via fasteners which may require the use of a sealant or caulk wherein the esters of the instant invention may also be advantageously employed.

A plastic film may also be applied with heat which includes calendaring, melt applications and shrink wrapping.

The composition may be part of a polish, such a furniture polish, or a dispersant or surfactant formulation such as a glycol or mineral oil dispersion or other formulation as used in for example wood protection.

Examples of useful surfactants include, but are not limited to, polyoxyethylene-based surface-active substances, including polyoxyethylene sorbitan tetraoleate (PST), poly-oxyethylene sorbitol hexaoleate (PSH), polyoxyethylene 6 tridecyl ether, polyoxyethyl-ene 12 tridecyl ether, polyoxyethylene 18 tridecyl ether, TWEEN® surfactants, TRITON® surfactants, and the polyoxyethlene-polyoxypropylene copolymers such as the PLURONIC® and POLOXAMER® product series (from BASF). Other matrix-forming components include dextrans, linear PEG molecules (MW 500 to 5,000,000), star-shaped PEG molecules, comb-shaped and dendrimeric, hyperbranched PEG molecules, as well as the analogous linear, star, and dendrimer polyamine polymers, and various carbonated, perfluorinated (e.g., DUPONT ZONYL® fluorosurfactants) and siliconated (e.g., dimethylsiloxane-ethylene oxide block copolymers) surfactants.

Given the wide array of applications for the present anti-microbial compositions, the composition may contain other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

The substrate can be an inorganic or organic substrate, for example, a metal or metal alloy; a thermoplastic, elastomeric, inherently crosslinked or crosslinked polymer as described above; a natural polymer such as wood or rubber; a ceramic material; glass; leather or other textile.

The substrate may be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

The inorganic or organic substrate is, for example, a metal or metal alloy, a thermoplastic, elastomeric, inherently crosslinked or crosslinked polymer, a ceramic material or a glass.

The substrate may be a multi-layered article comprised of the same or different components in each layer. The surface coated or laminated may be the exposed surface of an already applied coating or laminate.

The inorganic or organic substrate to be coated or laminated can be in any solid form.

For example, polymer substrates may be plastics in the form of films, injection-molded articles, extruded workpieces, fibres, felts or woven fabrics.

For example molded or extruded polymeric articles used in construction or the manufacture of durable goods such as siding, fascia and mailboxes can all benefit from the present method for stabilizer replenishment.

Plastics which would benefit from the present method include, but are not limited to, plastics used in construction or the manufacture of durable goods or machine parts, including outdoor furniture, boats, siding, roofing, glazing, protective films, decals, sealants, composites like plastic lumber and fiber reinforced composites, functional films including films used in displays as well as articles constructed from synthetic fibers such as awnings, fabrics such as used in canvas or sails and rubber articles such as outdoor matting and other uses cited in this disclosure. Exemplary of such plastics are polypropylene, polyethylene, PVC, POM, polysulfones, styrenics, polyamides, urethanes, polyesters, polycarbonate, acrylics, butadiene, thermoplastic polyolefins, ionomers, unsaturated polyesters and blends of polymer resins including ABS, SAN and PC/ABS.

The anti-microbial compounds of the invention are also effective in protecting useful plants, such as plants in agriculture, in horticulture and in forests, plant parts and seeds from disease and spoilage. For example, the present invention also provides a method which comprises applying to useful plants, the locus thereof or propagation material thereof a composition which comprises at least one of the polyglycerol polymers and co-polymers of the invention. Said compositions can be used as foliar, soil and seed treatment fungicides.

The compositions of the invention it is possible to inhibit or destroy the phytopathogenic microorganisms which occur in plants or in parts of plants (fruit, blossoms, leaves, stems, tubers, roots) in different useful plants. The present compositions are applied by treating the fungi, the useful plants, the locus thereof, the propagation material thereof, the natural substances of plant origin, which have been taken from the natural life cycle, and/or their processed forms, or the industrial materials threatened by fungus attack with the compositions in an effective amount.

The compositions according to the invention may be applied before or after infection of the useful plants, the propagation material thereof, the natural substances of plant and/or animal origin, which have been taken from the natural life cycle, and/or their processed forms, or the industrial materials by the fungi.

The compositions of the present invention are of particular interest for controlling a large number of fungi in various useful plants or their seeds, especially in field crops such as potatoes, tobacco and sugarbeets, and wheat, rye, barley, oats, rice, maize, lawns, cotton, soybeans, oil seed rape, pulse crops, sunflower, coffee, sugarcane, fruit and ornamentals in horticulture and viticulture, in vegetables such as cucumbers, beans and cucurbits.

When applied to plants, the anti-microbial compounds of the invention are applied at a rate of 1 to 5000 g a.i./ha, for example 2 to 2000 g a.i./ha, for example, 5 to 2000 g a.i./ha, for example, 10 to 1000 g a.i./ha, e.g. 50, 75, 100, 200, 250, 500, 800, 1000, 1500 g a.i./ha of polymer or co-polymers.

In agricultural practice the application rates depend on the type of effect desired, and typically range from 20 to 4000 g of total antimicrobials per hectare.

When treating seed, rates of 0.001 to 50 g of the present anti-microbial compounds, for example 0.01 to 10 g, per kg of seed, are generally sufficient.

The composition comprising the anti-microbial compounds of the invention may be employed in any conventional form, for example in the form a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). For example, formulations to be applied in spraying forms, such as water dispersible concentrates (e.g. EC, SC, DC, OD, SE, EW, EO and the like), wettable powders and granules, typically contain surfactants such as wetting and dispersing agents and other compounds that provide adjuvancy effects.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of at least one of the anti-microbial compounds, from 0 to 20% agriculturally acceptable surfactant and 10 to 99.99% solid or liquid formulation inerts and adjuvant(s), and optionally other active agents, particularly microbiocides or conservatives or the like. Concentrated forms of compositions generally contain in between about 2 and 80%, for example, between about 5 and 70% by weight of total active agent. Application forms of formulation may for example contain from 0.01 to 20% by weight, for example from 0.01 to 5% by weight of active agent.

Methods of preparing the above plant protection formulations are well known, for ex-ample, in US 20070265267, already incorporated by reference.

Particular embodiments of the invention therefore relate to methods for protecting plastics, coatings, other materials of construction, home or personal care formulations, plants, agricultural products, industrial formulations or technical process against the action of microbes which comprises adding an effective amount of the anti-microbial compounds of the present invention;

a method for protecting skin, mucosa and integumentary appendages against the action of microbes including protecting the scalp from dandruff, which comprises applying a preparation comprising an effective amount of the anti-microbial compounds of the present invention;

a method for protecting paper, wood, leather, synthetic textile materials or natural textile materials such as cotton against the action of microbes comprising incorporating or applying an effective amount of the present polymer or copolymer or a composition comprising an effective amount the anti-microbial compounds of the present invention;

a method for cleaning and disinfecting hard surfaces which comprises applying a preparation comprising an effective amount of the anti-microbial compounds of the present invention;

a method for preventing bio-fouling of an article comprising incorporating anti-microbial compounds of the present invention into the article or surface of the article or by applying the anti-microbial compounds of the present invention to these surfaces either directly or as part of a coating or film.

Other materials of construction include, in addition to wood, metals, paper, glass, ceramics, coatings, plastics and textiles, materials such as concrete, cement, adhesives, caulking materials, composites of natural and synthetic materials etc.

In the following examples for combinations of the compounds claimed in the present invention and biocides and/or preservatives are shown:

Table 2: Examples of combinations of biocides and/or preservatives used as antimicrobial actives in cosmetic products and household products

TABLE 2

| Combination name | | A | B | C | D | E | F | G | H | I | K | L | M | N | O | P | Q | R | S | T | U | V | W |
|---|---|---|---|---|---|---|---|---|---|---|---|---|---|---|---|---|---|---|---|---|---|---|---|
| BIOCIDE/PRESERVATIVE | 1 | x | | | | | | | | | | | | | | | | | | | | | |
| BIOCIDE/PRESERVATIVE | 2 | | x | | | | | | | | | | | | | | | | | | | | |
| BIOCIDE/PRESERVATIVE | 3 | | | x | | | | | | | | | | | | | | | | | | | |
| BIOCIDE/PRESERVATIVE | 4 | | | | x | | | | | | | | | | | | | | | | | | |
| BIOCIDE/PRESERVATIVE | 5 | | | | | x | | | | | | | | | | | | | | | | | |
| BIOCIDE/PRESERVATIVE | 6 | | | | | | x | | | | | | | | | | | | | | | | |
| BIOCIDE/PRESERVATIVE | 7 | | | | | | | x | | | | | | | | | | | | | | | |
| BIOCIDE/PRESERVATIVE | 8 | | | | | | | | x | | | | | | | | | | | | | | |
| BIOCIDE/PRESERVATIVE | 9 | | | | | | | | | x | | | | | | | | | | | | | |
| BIOCIDE/PRESERVATIVE | 10 | | | | | | | | | | x | | | | | | | | | | | | |
| BIOCIDE/PRESERVATIVE | 11 | | | | | | | | | | | x | | | | | | | | | | | |
| BIOCIDE/PRESERVATIVE | 12 | | | | | | | | | | | | x | | | | | | | | | | |
| BIOCIDE/PRESERVATIVE | 13 | | | | | | | | | | | | | x | | | | | | | | | |
| BIOCIDE/PRESERVATIVE | 14 | | | | | | | | | | | | | | x | | | | | | | | |
| BIOCIDE/PRESERVATIVE | 15 | | | | | | | | | | | | | | | x | | | | | | | |
| BIOCIDE/PRESERVATIVE | 16 | | | | | | | | | | | | | | | | x | | | | | | |
| BIOCIDE/PRESERVATIVE | 17 | | | | | | | | | | | | | | | | | x | | | | | |
| BIOCIDE/PRESERVATIVE | 18 | | | | | | | | | | | | | | | | | | x | | | | |
| BIOCIDE/PRESERVATIVE | 19 | | | | | | | | | | | | | | | | | | | x | | | |
| BIOCIDE/PRESERVATIVE | 20 | | | | | | | | | | | | | | | | | | | | x | | |
| BIOCIDE/PRESERVATIVE | 21 | | | | | | | | | | | | | | | | | | | | | x | |
| BIOCIDE/PRESERVATIVE | 22 | | | | | | | | | | | | | | | | | | | | | | x |
| BIOCIDE/PRESERVATIVE | 23 | x | x | | | | | | | | | | | | | | | | | | | | |
| BIOCIDE/PRESERVATIVE | 24 | x | | x | | | | | | | | | | | | | | | | | | | |
| BIOCIDE/PRESERVATIVE | 25 | x | | | x | | | | | | | | | | | | | | | | | | |
| BIOCIDE/PRESERVATIVE | 26 | x | | | | x | | | | | | | | | | | | | | | | | |
| BIOCIDE/PRESERVATIVE | 27 | x | | | | | x | | | | | | | | | | | | | | | | |
| BIOCIDE/PRESERVATIVE | 28 | x | | | | | | x | | | | | | | | | | | | | | | |
| BIOCIDE/PRESERVATIVE | 29 | x | | | | | | | x | | | | | | | | | | | | | | |
| BIOCIDE/PRESERVATIVE | 30 | x | | | | | | | | x | | | | | | | | | | | | | |
| BIOCIDE/PRESERVATIVE | 31 | x | | | | | | | | | x | | | | | | | | | | | | |
| BIOCIDE/PRESERVATIVE | 32 | x | | | | | | | | | | x | | | | | | | | | | | |
| BIOCIDE/PRESERVATIVE | 33 | x | | | | | | | | | | | x | | | | | | | | | | |
| BIOCIDE/PRESERVATIVE | 34 | x | | | | | | | | | | | | x | | | | | | | | | |
| BIOCIDE/PRESERVATIVE | 35 | x | | | | | | | | | | | | | x | | | | | | | | |
| BIOCIDE/PRESERVATIVE | 36 | x | | | | | | | | | | | | | | x | | | | | | | |
| BIOCIDE/PRESERVATIVE | 37 | x | | | | | | | | | | | | | | | x | | | | | | |
| BIOCIDE/PRESERVATIVE | 38 | x | | | | | | | | | | | | | | | | x | | | | | |
| BIOCIDE/PRESERVATIVE | 39 | x | | | | | | | | | | | | | | | | | x | | | | |
| BIOCIDE/PRESERVATIVE | 40 | x | | | | | | | | | | | | | | | | | | x | | | |
| BIOCIDE/PRESERVATIVE | 41 | x | | | | | | | | | | | | | | | | | | | x | | |
| BIOCIDE/PRESERVATIVE | 42 | x | | | | | | | | | | | | | | | | | | | | x | |
| BIOCIDE/PRESERVATIVE | 43 | x | | | | | | | | | | | | | | | | | | | | | x |
| BIOCIDE/PRESERVATIVE | 44 | | x | x | | | | | | | | | | | | | | | | | | | |
| BIOCIDE/PRESERVATIVE | 45 | | x | | x | | | | | | | | | | | | | | | | | | |
| BIOCIDE/PRESERVATIVE | 46 | | x | | | x | | | | | | | | | | | | | | | | | |
| BIOCIDE/PRESERVATIVE | 47 | | x | | | | x | | | | | | | | | | | | | | | | |
| BIOCIDE/PRESERVATIVE | 48 | | x | | | | | x | | | | | | | | | | | | | | | |
| BIOCIDE/PRESERVATIVE | 49 | | x | | | | | | x | | | | | | | | | | | | | | |
| BIOCIDE/PRESERVATIVE | 50 | | x | | | | | | | x | | | | | | | | | | | | | |
| BIOCIDE/PRESERVATIVE | 51 | | x | | | | | | | | x | | | | | | | | | | | | |
| BIOCIDE/PRESERVATIVE | 52 | | x | | | | | | | | | x | | | | | | | | | | | |
| BIOCIDE/PRESERVATIVE | 53 | | x | | | | | | | | | | x | | | | | | | | | | |
| BIOCIDE/PRESERVATIVE | 54 | | x | | | | | | | | | | | x | | | | | | | | | |
| BIOCIDE/PRESERVATIVE | 55 | | x | | | | | | | | | | | | x | | | | | | | | |
| BIOCIDE/PRESERVATIVE | 56 | | x | | | | | | | | | | | | | x | | | | | | | |
| BIOCIDE/PRESERVATIVE | 57 | | x | | | | | | | | | | | | | | x | | | | | | |
| BIOCIDE/PRESERVATIVE | 58 | | x | | | | | | | | | | | | | | | x | | | | | |
| BIOCIDE/PRESERVATIVE | 59 | | x | | | | | | | | | | | | | | | | x | | | | |
| BIOCIDE/PRESERVATIVE | 60 | | x | | | | | | | | | | | | | | | | | x | | | |
| BIOCIDE/PRESERVATIVE | 61 | | x | | | | | | | | | | | | | | | | | | x | | |
| BIOCIDE/PRESERVATIVE | 62 | | x | | | | | | | | | | | | | | | | | | | x | |
| BIOCIDE/PRESERVATIVE | 63 | | x | | | | | | | | | | | | | | | | | | | | x |
| BIOCIDE/PRESERVATIVE | 64 | | | x | x | | | | | | | | | | | | | | | | | | |
| BIOCIDE/PRESERVATIVE | 65 | | | x | | x | | | | | | | | | | | | | | | | | |
| BIOCIDE/PRESERVATIVE | 66 | | | x | | | x | | | | | | | | | | | | | | | | |
| BIOCIDE/PRESERVATIVE | 67 | | | x | | | | x | | | | | | | | | | | | | | | |
| BIOCIDE/PRESERVATIVE | 68 | | | x | | | | | x | | | | | | | | | | | | | | |
| BIOCIDE/PRESERVATIVE | 69 | | | x | | | | | | x | | | | | | | | | | | | | |
| BIOCIDE/PRESERVATIVE | 70 | | | x | | | | | | | x | | | | | | | | | | | | |
| BIOCIDE/PRESERVATIVE | 71 | | | x | | | | | | | | x | | | | | | | | | | | |
| BIOCIDE/PRESERVATIVE | 72 | | | x | | | | | | | | | x | | | | | | | | | | |
| BIOCIDE/PRESERVATIVE | 73 | | | x | | | | | | | | | | x | | | | | | | | | |
| BIOCIDE/PRESERVATIVE | 74 | | | x | | | | | | | | | | | x | | | | | | | | |
| BIOCIDE/PRESERVATIVE | 75 | | | x | | | | | | | | | | | | x | | | | | | | |
| BIOCIDE/PRESERVATIVE | 76 | | | x | | | | | | | | | | | | | x | | | | | | |
| BIOCIDE/PRESERVATIVE | 77 | | | x | | | | | | | | | | | | | | x | | | | | |
| BIOCIDE/PRESERVATIVE | 78 | | | x | | | | | | | | | | | | | | | x | | | | |
| BIOCIDE/PRESERVATIVE | 79 | | | x | | | | | | | | | | | | | | | | x | | | |
| BIOCIDE/PRESERVATIVE | 80 | | | x | | | | | | | | | | | | | | | | | x | | |
| BIOCIDE/PRESERVATIVE | 81 | | | x | | | | | | | | | | | | | | | | | | x | |
| BIOCIDE/PRESERVATIVE | 82 | | | x | | | | | | | | | | | | | | | | | | | x |
| BIOCIDE/PRESERVATIVE | 83 | | | | x | x | | | | | | | | | | | | | | | | | |
| BIOCIDE/PRESERVATIVE | 84 | | | | x | | x | | | | | | | | | | | | | | | | |
| BIOCIDE/PRESERVATIVE | 85 | | | | x | | | x | | | | | | | | | | | | | | | |
| BIOCIDE/PRESERVATIVE | 86 | | | | x | | | | x | | | | | | | | | | | | | | |
| BIOCIDE/PRESERVATIVE | 87 | | | | x | | | | | x | | | | | | | | | | | | | |
| BIOCIDE/PRESERVATIVE | 88 | | | | x | | | | | | x | | | | | | | | | | | | |
| BIOCIDE/PRESERVATIVE | 89 | | | | x | | | | | | | x | | | | | | | | | | | |
| BIOCIDE/PRESERVATIVE | 90 | | | | x | | | | | | | | x | | | | | | | | | | |
| BIOCIDE/PRESERVATIVE | 91 | | | | x | | | | | | | | | x | | | | | | | | | |
| BIOCIDE/PRESERVATIVE | 92 | | | | x | | | | | | | | | | x | | | | | | | | |
| BIOCIDE/PRESERVATIVE | 93 | | | | x | | | | | | | | | | | x | | | | | | | |
| BIOCIDE/PRESERVATIVE | 94 | | | | x | | | | | | | | | | | | x | | | | | | |
| BIOCIDE/PRESERVATIVE | 95 | | | | x | | | | | | | | | | | | | x | | | | | |
| BIOCIDE/PRESERVATIVE | 96 | | | | x | | | | | | | | | | | | | | x | | | | |
| BIOCIDE/PRESERVATIVE | 97 | | | | x | | | | | | | | | | | | | | | x | | | |
| BIOCIDE/PRESERVATIVE | 98 | | | | x | | | | | | | | | | | | | | | | x | | |
| BIOCIDE/PRESERVATIVE | 99 | | | | x | | | | | | | | | | | | | | | | | x | |
| BIOCIDE/PRESERVATIVE | 100 | | | | x | | | | | | | | | | | | | | | | | | x |
| BIOCIDE/PRESERVATIVE | 101 | | | | | x | x | | | | | | | | | | | | | | | | |
| BIOCIDE/PRESERVATIVE | 102 | | | | | x | | x | | | | | | | | | | | | | | | |
| BIOCIDE/PRESERVATIVE | 103 | | | | | x | | | x | | | | | | | | | | | | | | |
| BIOCIDE/PRESERVATIVE | 104 | | | | | x | | | | x | | | | | | | | | | | | | |
| BIOCIDE/PRESERVATIVE | 105 | | | | | x | | | | | x | | | | | | | | | | | | |
| BIOCIDE/PRESERVATIVE | 106 | | | | | x | | | | | | x | | | | | | | | | | | |
| BIOCIDE/PRESERVATIVE | 107 | | | | | x | | | | | | | x | | | | | | | | | | |
| BIOCIDE/PRESERVATIVE | 108 | | | | | x | | | | | | | | x | | | | | | | | | |
| BIOCIDE/PRESERVATIVE | 109 | | | | | x | | | | | | | | | x | | | | | | | | |
| BIOCIDE/PRESERVATIVE | 110 | | | | | x | | | | | | | | | | x | | | | | | | |
| BIOCIDE/PRESERVATIVE | 111 | | | | | x | | | | | | | | | | | x | | | | | | |
| BIOCIDE/PRESERVATIVE | 112 | | | | | x | | | | | | | | | | | | x | | | | | |
| BIOCIDE/PRESERVATIVE | 113 | | | | | x | | | | | | | | | | | | | x | | | | |
| BIOCIDE/PRESERVATIVE | 114 | | | | | x | | | | | | | | | | | | | | x | | | |
| BIOCIDE/PRESERVATIVE | 115 | | | | | x | | | | | | | | | | | | | | | x | | |
| BIOCIDE/PRESERVATIVE | 116 | | | | | x | | | | | | | | | | | | | | | | x | |
| BIOCIDE/PRESERVATIVE | 117 | | | | | x | | | | | | | | | | | | | | | | | x |
| BIOCIDE/PRESERVATIVE | 118 | | | | | | x | x | | | | | | | | | | | | | | | |
| BIOCIDE/PRESERVATIVE | 119 | | | | | | x | | x | | | | | | | | | | | | | | |
| BIOCIDE/PRESERVATIVE | 120 | | | | | | x | | | x | | | | | | | | | | | | | |
| BIOCIDE/PRESERVATIVE | 121 | | | | | | x | | | | x | | | | | | | | | | | | |
| BIOCIDE/PRESERVATIVE | 122 | | | | | | x | | | | | x | | | | | | | | | | | |
| BIOCIDE/PRESERVATIVE | 123 | | | | | | x | | | | | | x | | | | | | | | | | |
| BIOCIDE/PRESERVATIVE | 124 | | | | | | x | | | | | | | x | | | | | | | | | |
| BIOCIDE/PRESERVATIVE | 125 | | | | | | x | | | | | | | | x | | | | | | | | |
| BIOCIDE/PRESERVATIVE | 126 | | | | | | x | | | | | | | | | x | | | | | | | |
| BIOCIDE/PRESERVATIVE | 127 | | | | | | x | | | | | | | | | | x | | | | | | |
| BIOCIDE/PRESERVATIVE | 128 | | | | | | x | | | | | | | | | | | x | | | | | |
| BIOCIDE/PRESERVATIVE | 129 | | | | | | x | | | | | | | | | | | | x | | | | |
| BIOCIDE/PRESERVATIVE | 130 | | | | | | x | | | | | | | | | | | | | x | | | |
| BIOCIDE/PRESERVATIVE | 131 | | | | | | x | | | | | | | | | | | | | | x | | |
| BIOCIDE/PRESERVATIVE | 132 | | | | | | x | | | | | | | | | | | | | | | x | |
| BIOCIDE/PRESERVATIVE | 133 | | | | | | x | | | | | | | | | | | | | | | | x |
| BIOCIDE/PRESERVATIVE | 134 | | | | | | | x | x | | | | | | | | | | | | | | |
| BIOCIDE/PRESERVATIVE | 135 | | | | | | | x | | x | | | | | | | | | | | | | |
| BIOCIDE/PRESERVATIVE | 136 | | | | | | | x | | | x | | | | | | | | | | | | |
| BIOCIDE/PRESERVATIVE | 137 | | | | | | | x | | | | x | | | | | | | | | | | |
| BIOCIDE/PRESERVATIVE | 138 | | | | | | | x | | | | | x | | | | | | | | | | |
| BIOCIDE/PRESERVATIVE | 139 | | | | | | | x | | | | | | x | | | | | | | | | |
| BIOCIDE/PRESERVATIVE | 140 | | | | | | | x | | | | | | | x | | | | | | | | |
| BIOCIDE/PRESERVATIVE | 141 | | | | | | | x | | | | | | | | x | | | | | | | |
| BIOCIDE/PRESERVATIVE | 142 | | | | | | | x | | | | | | | | | x | | | | | | |
| BIOCIDE/PRESERVATIVE | 143 | | | | | | | x | | | | | | | | | | x | | | | | |
| BIOCIDE/PRESERVATIVE | 144 | | | | | | | x | | | | | | | | | | | x | | | | |
| BIOCIDE/PRESERVATIVE | 145 | | | | | | | x | | | | | | | | | | | | x | | | |
| BIOCIDE/PRESERVATIVE | 146 | | | | | | | x | | | | | | | | | | | | | x | | |
| BIOCIDE/PRESERVATIVE | 147 | | | | | | | x | | | | | | | | | | | | | | x | |
| BIOCIDE/PRESERVATIVE | 148 | | | | | | | x | | | | | | | | | | | | | | | x |
| BIOCIDE/PRESERVATIVE | 149 | | | | | | | | x | x | | | | | | | | | | | | | |
| BIOCIDE/PRESERVATIVE | 150 | | | | | | | | x | | x | | | | | | | | | | | | |
| BIOCIDE/PRESERVATIVE | 151 | | | | | | | | x | | | x | | | | | | | | | | | |
| BIOCIDE/PRESERVATIVE | 152 | | | | | | | | x | | | | x | | | | | | | | | | |
| BIOCIDE/PRESERVATIVE | 153 | | | | | | | | x | | | | | x | | | | | | | | | |
| BIOCIDE/PRESERVATIVE | 154 | | | | | | | | x | | | | | | x | | | | | | | | |
| BIOCIDE/PRESERVATIVE | 155 | | | | | | | | x | | | | | | | x | | | | | | | |
| BIOCIDE/PRESERVATIVE | 156 | | | | | | | | x | | | | | | | | x | | | | | | |
| BIOCIDE/PRESERVATIVE | 157 | | | | | | | | x | | | | | | | | | x | | | | | |
| BIOCIDE/PRESERVATIVE | 158 | | | | | | | | x | | | | | | | | | | x | | | | |
| BIOCIDE/PRESERVATIVE | 159 | | | | | | | | x | | | | | | | | | | | x | | | |
| BIOCIDE/PRESERVATIVE | 160 | | | | | | | | x | | | | | | | | | | | | x | | |
| BIOCIDE/PRESERVATIVE | 161 | | | | | | | | x | | | | | | | | | | | | | x | |
| BIOCIDE/PRESERVATIVE | 162 | | | | | | | | x | | | | | | | | | | | | | | x |
| BIOCIDE/PRESERVATIVE | 163 | | | | | | | | | x | x | | | | | | | | | | | | |
| BIOCIDE/PRESERVATIVE | 164 | | | | | | | | | x | | x | | | | | | | | | | | |
| BIOCIDE/PRESERVATIVE | 165 | | | | | | | | | x | | | x | | | | | | | | | | |
| BIOCIDE/PRESERVATIVE | 166 | | | | | | | | | x | | | | x | | | | | | | | | |
| BIOCIDE/PRESERVATIVE | 167 | | | | | | | | | x | | | | | x | | | | | | | | |
| BIOCIDE/PRESERVATIVE | 168 | | | | | | | | | x | | | | | | x | | | | | | | |
| BIOCIDE/PRESERVATIVE | 169 | | | | | | | | | x | | | | | | | x | | | | | | |
| BIOCIDE/PRESERVATIVE | 170 | | | | | | | | | x | | | | | | | | x | | | | | |
| BIOCIDE/PRESERVATIVE | 171 | | | | | | | | | x | | | | | | | | | x | | | | |
| BIOCIDE/PRESERVATIVE | 172 | | | | | | | | | x | | | | | | | | | | x | | | |
| BIOCIDE/PRESERVATIVE | 173 | | | | | | | | | x | | | | | | | | | | | x | | |
| BIOCIDE/PRESERVATIVE | 174 | | | | | | | | | x | | | | | | | | | | | | x | |
| BIOCIDE/PRESERVATIVE | 175 | | | | | | | | | x | | | | | | | | | | | | | x |
| BIOCIDE/PRESERVATIVE | 176 | | | | | | | | | | x | x | | | | | | | | | | | |
| BIOCIDE/PRESERVATIVE | 177 | | | | | | | | | | x | | x | | | | | | | | | | |
| BIOCIDE/PRESERVATIVE | 178 | | | | | | | | | | x | | | x | | | | | | | | | |
| BIOCIDE/PRESERVATIVE | 179 | | | | | | | | | | x | | | | x | | | | | | | | |
| BIOCIDE/PRESERVATIVE | 180 | | | | | | | | | | x | | | | | x | | | | | | | |
| BIOCIDE/PRESERVATIVE | 181 | | | | | | | | | | x | | | | | | x | | | | | | |
| BIOCIDE/PRESERVATIVE | 182 | | | | | | | | | | x | | | | | | | x | | | | | |
| BIOCIDE/PRESERVATIVE | 183 | | | | | | | | | | x | | | | | | | | x | | | | |
| BIOCIDE/PRESERVATIVE | 184 | | | | | | | | | | x | | | | | | | | | x | | | |
| BIOCIDE/PRESERVATIVE | 185 | | | | | | | | | | x | | | | | | | | | | x | | |
| BIOCIDE/PRESERVATIVE | 186 | | | | | | | | | | x | | | | | | | | | | | x | |
| BIOCIDE/PRESERVATIVE | 187 | | | | | | | | | | x | | | | | | | | | | | | x |
| BIOCIDE/PRESERVATIVE | 188 | | | | | | | | | | | x | x | | | | | | | | | | |
| BIOCIDE/PRESERVATIVE | 189 | | | | | | | | | | | x | | x | | | | | | | | | |
| BIOCIDE/PRESERVATIVE | 190 | | | | | | | | | | | x | | | x | | | | | | | | |
| BIOCIDE/PRESERVATIVE | 191 | | | | | | | | | | | x | | | | x | | | | | | | |
| BIOCIDE/PRESERVATIVE | 192 | | | | | | | | | | | x | | | | | x | | | | | | |
| BIOCIDE/PRESERVATIVE | 193 | | | | | | | | | | | x | | | | | | x | | | | | |
| BIOCIDE/PRESERVATIVE | 194 | | | | | | | | | | | x | | | | | | | x | | | | |
| BIOCIDE/PRESERVATIVE | 195 | | | | | | | | | | | x | | | | | | | | x | | | |
| BIOCIDE/PRESERVATIVE | 196 | | | | | | | | | | | x | | | | | | | | | x | | |
| BIOCIDE/PRESERVATIVE | 197 | | | | | | | | | | | x | | | | | | | | | | x | |
| BIOCIDE/PRESERVATIVE | 198 | | | | | | | | | | | x | | | | | | | | | | | x |
| BIOCIDE/PRESERVATIVE | 199 | | | | | | | | | | | | x | x | | | | | | | | | |
| BIOCIDE/PRESERVATIVE | 200 | | | | | | | | | | | | x | | x | | | | | | | | |
| BIOCIDE/PRESERVATIVE | 201 | | | | | | | | | | | | x | | | x | | | | | | | |
| BIOCIDE/PRESERVATIVE | 202 | | | | | | | | | | | | x | | | | x | | | | | | |
| BIOCIDE/PRESERVATIVE | 203 | | | | | | | | | | | | x | | | | | x | | | | | |
| BIOCIDE/PRESERVATIVE | 204 | | | | | | | | | | | | x | | | | | | x | | | | |
| BIOCIDE/PRESERVATIVE | 205 | | | | | | | | | | | | x | | | | | | | x | | | |
| BIOCIDE/PRESERVATIVE | 206 | | | | | | | | | | | | x | | | | | | | | x | | |
| BIOCIDE/PRESERVATIVE | 207 | | | | | | | | | | | | x | | | | | | | | | x | |
| BIOCIDE/PRESERVATIVE | 208 | | | | | | | | | | | | x | | | | | | | | | | x |
| BIOCIDE/PRESERVATIVE | 209 | | | | | | | | | | | | | x | x | | | | | | | | |
| BIOCIDE/PRESERVATIVE | 210 | | | | | | | | | | | | | x | | x | | | | | | | |
| BIOCIDE/PRESERVATIVE | 211 | | | | | | | | | | | | | x | | | x | | | | | | |
| BIOCIDE/PRESERVATIVE | 212 | | | | | | | | | | | | | x | | | | x | | | | | |
| BIOCIDE/PRESERVATIVE | 213 | | | | | | | | | | | | | x | | | | | x | | | | |
| BIOCIDE/PRESERVATIVE | 214 | | | | | | | | | | | | | x | | | | | | x | | | |
| BIOCIDE/PRESERVATIVE | 215 | | | | | | | | | | | | | x | | | | | | | x | | |
| BIOCIDE/PRESERVATIVE | 216 | | | | | | | | | | | | | x | | | | | | | | x | |
| BIOCIDE/PRESERVATIVE | 217 | | | | | | | | | | | | | x | | | | | | | | | x |
| BIOCIDE/PRESERVATIVE | 218 | | | | | | | | | | | | | | x | x | | | | | | | |
| BIOCIDE/PRESERVATIVE | 219 | | | | | | | | | | | | | | x | | x | | | | | | |
| BIOCIDE/PRESERVATIVE | 220 | | | | | | | | | | | | | | x | | | x | | | | | |
| BIOCIDE/PRESERVATIVE | 221 | | | | | | | | | | | | | | x | | | | x | | | | |
| BIOCIDE/PRESERVATIVE | 222 | | | | | | | | | | | | | | x | | | | | x | | | |
| BIOCIDE/PRESERVATIVE | 223 | | | | | | | | | | | | | | x | | | | | | x | | |
| BIOCIDE/PRESERVATIVE | 224 | | | | | | | | | | | | | | x | | | | | | | x | |
| BIOCIDE/PRESERVATIVE | 225 | | | | | | | | | | | | | | x | | | | | | | | x |
| BIOCIDE/PRESERVATIVE | 226 | | | | | | | | | | | | | | | x | x | | | | | | |
| BIOCIDE/PRESERVATIVE | 227 | | | | | | | | | | | | | | | x | | x | | | | | |
| BIOCIDE/PRESERVATIVE | 228 | | | | | | | | | | | | | | | x | | | x | | | | |
| BIOCIDE/PRESERVATIVE | 229 | | | | | | | | | | | | | | | x | | | | x | | | |
| BIOCIDE/PRESERVATIVE | 230 | | | | | | | | | | | | | | | x | | | | | x | | |
| BIOCIDE/PRESERVATIVE | 231 | | | | | | | | | | | | | | | x | | | | | | x | |
| BIOCIDE/PRESERVATIVE | 232 | | | | | | | | | | | | | | | x | | | | | | | x |
| BIOCIDE/PRESERVATIVE | 233 | | | | | | | | | | | | | | | | x | x | | | | | |
| BIOCIDE/PRESERVATIVE | 234 | | | | | | | | | | | | | | | | x | | x | | | | |
| BIOCIDE/PRESERVATIVE | 235 | | | | | | | | | | | | | | | | x | | | x | | | |
| BIOCIDE/PRESERVATIVE | 236 | | | | | | | | | | | | | | | | x | | | | x | | |
| BIOCIDE/PRESERVATIVE | 237 | | | | | | | | | | | | | | | | x | | | | | x | |
| BIOCIDE/PRESERVATIVE | 238 | | | | | | | | | | | | | | | | x | | | | | | x |
| BIOCIDE/PRESERVATIVE | 239 | | | | | | | | | | | | | | | | | x | x | | | | |
| BIOCIDE/PRESERVATIVE | 240 | | | | | | | | | | | | | | | | | x | | x | | | |
| BIOCIDE/PRESERVATIVE | 241 | | | | | | | | | | | | | | | | | x | | | x | | |
| BIOCIDE/PRESERVATIVE | 242 | | | | | | | | | | | | | | | | | x | | | | x | |
| BIOCIDE/PRESERVATIVE | 243 | | | | | | | | | | | | | | | | | x | | | | | x |
| BIOCIDE/PRESERVATIVE | 244 | | | | | | | | | | | | | | | | | | x | x | | | |
| BIOCIDE/PRESERVATIVE | 245 | | | | | | | | | | | | | | | | | | x | | x | | |
| BIOCIDE/PRESERVATIVE | 246 | | | | | | | | | | | | | | | | | | x | | | x | |
| BIOCIDE/PRESERVATIVE | 247 | | | | | | | | | | | | | | | | | | x | | | | x |
| BIOCIDE/PRESERVATIVE | 248 | | | | | | | | | | | | | | | | | | | x | x | | |
| BIOCIDE/PRESERVATIVE | 249 | | | | | | | | | | | | | | | | | | | x | | x | |
| BIOCIDE/PRESERVATIVE | 250 | | | | | | | | | | | | | | | | | | | x | | | x |
| BIOCIDE/PRESERVATIVE | 251 | | | | | | | | | | | | | | | | | | | | x | x | |
| BIOCIDE/PRESERVATIVE | 252 | | | | | | | | | | | | | | | | | | | | x | | x |
| BIOCIDE/PRESERVATIVE | 253 | | | | | | | | | | | | | | | | | | | | | x | x |

| | INCI Name | CAS No. |
|---|---|---|
| A | Benzoic acid or/and its salts | 65-85-0 |
| B | 2-bromo-2-nitropropane-1,3-diol | 52-51-7 |
| C | Benzyl alcohol | 100-51-6 |
| D | Benzisothiazolinone | 2634-33-5 |
| E | Dehydroacetic acid | 16807-48-0 |
| F | Dichlorobenzyl alcohol | 12041-76-8 |
| G | DMDM hydantoin | 6440-58-0 |
| H | Diazolidinyl urea | 78491-02-8 |
| I | Ethylparaben | 120-47-8 |
| K | Iodopropynyl butylcarbamate | 55406-53-6 |
| L | Imidazolidinyl urea | 39236-46-9 |
| M | Methylchloroisothiazolinone | 26172-55-4 |
| N | Methyldibromo glutaronitrile | 35691-65-7 |
| O | Methylparaben | 99-76-3 |
| P | Methylisothiazolinone | 2682-20-4 |
| Q | 2-phenoxyethanol | 122-99-6 |
| R | Propylparaben | 94-13-3 |
| S | Salicylic acid or/and its salts | 69-72-7 |
| T | Sorbic acid and/or its salts | 110-44-1 |
| U | Triclosan | 3380-34-5 |
| V | Undecylenic acid and/or its salts | 1333-28-4 |
| W | Silver Citrate and Citric acid | 36701-38-9 |

In all of the biocide combinations listed before in Table 2, “X” represents a specific biocide and/or preservative. The concentrations of these biocidal/preservative substances in the blends are between 1% and 99%, preferably between 10% and 90%. The use concentration of these blends in household and personal care products is typically in the range of 0.1% to 5% and preferred 0.2% to 2%.

Further biocide combination examples are the following:

In all the biocide combinations listed before the weight ratio of the biocides and or preservatives (based on the weight of all biocides/preservatives in the combination) is for combinations of two biocides/preservatives 1:1 or 1:2 or 2:1 or 1:3 or 3:1.

### FORMULATION EXAMPLES

The compounds of the present invention from table 1 can be compound with the BIOCIDES/PRESERVATIVES from Table 2 as shown in the following formulation examples:

“BIOCIDE/PRESERVATIVE” may be (as described in Table 2) BIOCIDE/PRESERVATIVE 1, or BIOCIDE/PRESERVATIVE 2, or BIOCIDE/PRESERVATIVE 3, or BIOCIDE/PRESERVATIVE 4, or BIOCIDE/PRESERVATIVE 5, or BIOCIDE/PRESERVATIVE 6, or BIOCIDE/PRESERVATIVE 7, or BIOCIDE/PRESERVATIVE 8, or BIOCIDE/PRESERVATIVE 9, or BIOCIDE/PRESERVATIVE 10, or BIOCIDE/PRESERVATIVE 11, or BIOCIDE/PRESERVATIVE 12, or BIOCIDE/PRESERVATIVE 13, or BIOCIDE/PRESERVATIVE 14, or BIOCIDE/PRESERVATIVE 15, or BIOCIDE/PRESERVATIVE 16, or BIOCIDE/PRESERVATIVE 17, or BIOCIDE/PRESERVATIVE 18, or BIOCIDE/PRESERVATIVE 19, or BIOCIDE/PRESERVATIVE 20, or BIOCIDE/PRESERVATIVE 21, or BIOCIDE/PRESERVATIVE 22, or BIOCIDE/PRESERVATIVE 23, or BIOCIDE/PRESERVATIVE 24, or BIOCIDE/PRESERVATIVE 25, or BIOCIDE/PRESERVATIVE 26, or BIOCIDE/PRESERVATIVE 27, or BIOCIDE/PRESERVATIVE 28, or BIOCIDE/PRESERVATIVE 29, or BIOCIDE/PRESERVATIVE 30, or BIOCIDE/PRESERVATIVE 31, or BIOCIDE/PRESERVATIVE 32, or BIOCIDE/PRESERVATIVE 33, or BIOCIDE/PRESERVATIVE 34, or BIOCIDE/PRESERVATIVE 35, or BIOCIDE/PRESERVATIVE 36, or BIOCIDE/PRESERVATIVE 37, or BIOCIDE/PRESERVATIVE 38, or BIOCIDE/PRESERVATIVE 39, or BIOCIDE/PRESERVATIVE 40, or BIOCIDE/PRESERVATIVE 41, or BIOCIDE/PRESERVATIVE 42, or BIOCIDE/PRESERVATIVE 43, or BIOCIDE/-PRESERVATIVE 44, or BIOCIDE/PRESERVATIVE 45, or BIOCIDE/PRESERVATIVE 46, or BIOCIDE/PRESERVATIVE 47, or BIOCIDE/PRESERVATIVE 48, or BIOCIDE/PRESERVATIVE 49, or BIOCIDE/PRESERVATIVE 50, or BIOCIDE/PRESERVATIVE 51, or BIOCIDE/PRESERVATIVE 52, or BIOCIDE/PRESERVATIVE 53, or BIOCIDE/PRESERVATIVE 54, or BIOCIDE/PRESERVATIVE 55, or BIOCIDE/PRESERVATIVE 56, or BIOCIDE/PRESERVATIVE 57, or BIOCIDE/PRESERVATIVE 58, or BIOCIDE/PRESERVATIVE 59, or BIOCIDE/PRESERVATIVE 60, or BIOCIDE/PRESERVATIVE 61, or BIOCIDE/PRESERVATIVE 62, or BIOCIDE/PRESERVATIVE 63, or BIOCIDE/PRESERVATIVE 64, or BIOCIDE/PRESERVATIVE 65, or BIOCIDE/PRESERVATIVE 66, or BIOCIDE/PRESERVATIVE 67, or BIOCIDE/-PRESERVATIVE 68, or BIOCIDE/PRESERVATIVE 69, or BIOCIDE/PRESERVATIVE 70, or BIOCIDE/PRESERVATIVE 71, or BIOCIDE/PRESERVATIVE 72, or BIOCIDE/PRESERVATIVE 73, or BIOCIDE/PRESERVATIVE 74, or BIOCIDE/PRESERVATIVE 75, or BIOCIDE/PRESERVATIVE 76, or BIOCIDE/PRESERVATIVE 77, or BIOCIDE/PRESERVATIVE 78, or BIOCIDE/PRESERVATIVE 79, or BIOCIDE/PRESERVATIVE 80, or BIOCIDE/PRESERVATIVE 81, or BIOCIDE/PRESERVATIVE 82, or BIOCIDE/PRESERVATIVE 83, or BIOCIDE/PRESERVATIVE 84, or BIOCIDE/PRESERVATIVE 85, or BIOCIDE/PRESERVATIVE 86, or BIOCIDE/PRESERVATIVE 87, or BIOCIDE/PRESERVATIVE 88, or BIOCIDE/PRESERVATIVE 89, or BIOCIDE/PRESERVATIVE 90, or BIOCIDE/PRESERVATIVE 91, or BIOCIDE/PRESERVATIVE 92, or BIOCIDE/PRESERVATIVE 93, or BIOCIDE/PRESERVATIVE 94, or BIOCIDE/PRESERVATIVE 95, or BIOCIDE/PRESERVATIVE 96, or BIOCIDE/PRESERVATIVE 97, or BIOCIDE/PRESERVATIVE 98, or BIOCIDE/PRESERVATIVE 99, or BIOCIDE/PRESERVATIVE 100, or BIOCIDE/PRESERVATIVE 101, or BIOCIDE/PRESERVATIVE 102, or BIOCIDE/PRESERVATIVE 103, or BIOCIDE/PRESERVATIVE 104, or BIOCIDE/PRESERVATIVE 105, or BIOCIDE/PRESERVATIVE 106, or BIOCIDE/PRESERVATIVE 107, or BIOCIDE/PRESERVATIVE 108, or BIOCIDE/PRESERVATIVE 109, or BIOCIDE/PRESERVATIVE 110, or BIOCIDE/PRESERVATIVE 111, or BIOCIDE/PRESERVATIVE 112, or BIOCIDE/PRESERVATIVE 113, or BIOCIDE/PRESERVATIVE 114, or BIOCIDE/PRESERVATIVE 115, or BIOCIDE/PRESERVATIVE 116, or BIOCIDE/PRESERVATIVE 117, or BIOCIDE/PRESERVATIVE 118, or BIOCIDE/PRESERVATIVE 119, or BIOCIDE/PRESERVATIVE 120, or BIOCIDE/PRESERVATIVE 121, or BIOCIDE/PRESERVATIVE 122, or BIOCIDE/PRESERVATIVE 123, or BIOCIDE/PRESERVATIVE 124, or BIOCIDE/PRESERVATIVE 125, or BIOCIDE/PRESERVATIVE 126, or BIOCIDE/PRESERVATIVE 127, or BIOCIDE/PRESERVATIVE 128, or BIOCIDE/PRESERVATIVE 129, or BIOCIDE/PRESERVATIVE 130, or BIOCIDE/PRESERVATIVE 131, or BIOCIDE/PRESERVATIVE 132, or BIOCIDE/PRESERVATIVE 133, or BIOCIDE/PRESERVATIVE 134, or BIOCIDE/PRESERVATIVE 135, or BIOCIDE/PRESERVATIVE 136, or BIOCIDE/PRESERVATIVE 137, or BIOCIDE/PRESERVATIVE 138, or BIOCIDE/PRESERVATIVE 139, or BIOCIDE/PRESERVATIVE 140, or BIOCIDE/PRESERVATIVE 141, or BIOCIDE/PRESERVATIVE 142, or BIOCIDE/PRESERVATIVE 143, or BIOCIDE/PRESERVATIVE 144, or BIOCIDE/PRESERVATIVE 145, or BIOCIDE/PRESERVATIVE 146, or BIOCIDE/PRESERVATIVE 147, or BIOCIDE/PRESERVATIVE 148, or BIOCIDE/PRESERVATIVE 149, or BIOCIDE/PRESERVATIVE 150, or BIOCIDE/PRESERVATIVE 151, or BIOCIDE/PRESERVATIVE 152, or BIOCIDE/PRESERVATIVE 153, or BIOCIDE/PRESERVATIVE 154, or BIOCIDE/PRESERVATIVE 155, or BIOCIDE/PRESERVATIVE 156, or BIOCIDE/PRESERVATIVE 157, or BIOCIDE/PRESERVATIVE 158, or BIOCIDE/PRESERVATIVE 159, or BIOCIDE/PRESERVATIVE 160, or BIOCIDE/PRESERVATIVE 161, or BIOCIDE/PRESERVATIVE 162, or BIOCIDE/PRESERVATIVE 163, or BIOCIDE/PRESERVATIVE 164, or BIOCIDE/PRESERVATIVE 165, or BIOCIDE/PRESERVATIVE 166, or BIOCIDE/PRESERVATIVE 167, or BIOCIDE/PRESERVATIVE 168, or BIOCIDE/PRESERVATIVE 169, or BIOCIDE/PRESERVATIVE 170, or BIOCIDE/PRESERVATIVE 171, or BIOCIDE/-PRESERVATIVE 172, or BIOCIDE/PRESERVATIVE 173, or BIOCIDE/PRESERVATIVE 174, or BIOCIDE/PRESERVATIVE 175, or BIOCIDE/PRESERVATIVE 176, or BIOCIDE/PRESERVATIVE 177, or BIOCIDE/PRESERVATIVE 178, or BIOCIDE/PRESERVATIVE 179, or BIOCIDE/PRESERVATIVE 180, or BIOCIDE/PRESERVATIVE 181, or BIOCIDE/PRESERVATIVE 182, or BIOCIDE/PRESERVATIVE 183, or BIOCIDE/PRESERVATIVE 184, or BIOCIDE/PRESERVATIVE 185, or BIOCIDE/PRESERVATIVE 186, or BIOCIDE/-PRESERVATIVE 187, or BIOCIDE/PRESERVATIVE 188, or BIOCIDE/PRESERVATIVE 189, or BIOCIDE/PRESERVATIVE 190, or BIOCIDE/PRESERVATIVE 191, or BIOCIDE/PRESERVATIVE 192, or BIOCIDE/PRESERVATIVE 193, or BIOCIDE/PRESERVATIVE 194, or BIOCIDE/PRESERVATIVE 195, or BIOCIDE/PRESERVATIVE 196, or BIOCIDE/PRESERVATIVE 197, or BIOCIDE/PRESERVATIVE 198, or BIOCIDE/PRESERVATIVE 199, or BIOCIDE/PRESERVATIVE 200, or BIOCIDE/PRESERVATIVE 201, or BIOCIDE/-PRESERVATIVE 202, or BIOCIDE/PRESERVATIVE 203, or BIOCIDE/PRESERVATIVE 204, or BIOCIDE/PRESERVATIVE 205, or BIOCIDE/PRESERVATIVE 206, or BIOCIDE/PRESERVATIVE 207, or BIOCIDE/PRESERVATIVE 208, or BIOCIDE/PRESERVATIVE 209, or BIOCIDE/PRESERVATIVE 210, or BIOCIDE/PRESERVATIVE 211, or BIOCIDE/PRESERVATIVE 212, or BIOCIDE/PRESERVATIVE 213, or BIOCIDE/PRESERVATIVE 214, or BIOCIDE/PRESERVATIVE 215, or BIOCIDE/PRESERVATIVE 216, or BIOCIDE/PRESERVATIVE 217, or BIOCIDE/PRESERVATIVE 218, or BIOCIDE/PRESERVATIVE 219, or BIOCIDE/PRESERVATIVE 220, or BIOCIDE/PRESERVATIVE 221, or BIOCIDE/PRESERVATIVE 222, or BIOCIDE/PRESERVATIVE 223, or BIOCIDE/PRESERVATIVE 224, or BIOCIDE/PRESERVATIVE 225, or BIOCIDE/PRESERVATIVE 226, or BIOCIDE/PRESERVATIVE 227, or BIOCIDE/PRESERVATIVE 228, or BIOCIDE/PRESERVATIVE 229, or BIOCIDE/PRESERVATIVE 230, or BIOCIDE/PRESERVATIVE 231, or BIOCIDE/PRESERVATIVE 232, or BIOCIDE/PRESERVATIVE 233, or BIOCIDE/PRESERVATIVE 234, or BIOCIDE/PRESERVATIVE 235, or BIOCIDE/PRESERVATIVE 236, or BIOCIDE/PRESERVATIVE 237, or BIOCIDE/PRESERVATIVE 238, or BIOCIDE/PRESERVATIVE 239, or BIOCIDE/PRESERVATIVE 240, or BIOCIDE/PRESERVATIVE 241, or BIOCIDE/PRESERVATIVE 242, or BIOCIDE/PRESERVATIVE 243, or BIOCIDE/PRESERVATIVE 244, or BIOCIDE/PRESERVATIVE 245, or BIOCIDE/PRESERVATIVE 246, or BIOCIDE/PRESERVATIVE 247, or BIOCIDE/PRESERVATIVE 248, or BIOCIDE/PRESERVATIVE 249, or BIOCIDE/PRESERVATIVE 250, or BIOCIDE/PRESERVATIVE 251, or BIOCIDE/PRESERVATIVE 252, or BIOCIDE/PRESERVATIVE 253.

In the following some examples of formulations combining the compounds of the present invention with other cosmetic and household product ingredients including the BIOCIDES/PRESERVATIVES of table 2 are shown:

Sun Milk

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| C12-15 Alkyl Benzoate | 2.00 | 2.00 | 2.00 | 2.00 | 2.00 | 2.00 | 2.00 | 2.00 | 2.00 |
| Phenethyl Benzoate | 4.00 | 4.00 | 4.00 | 4.00 | 4.00 | 4.00 | 4.00 | 4.00 | 4.00 |
| Tridecyl Trimellitate | 3.00 | 3.00 | 3.00 | 3.00 | 3.00 | 3.00 | 3.00 | 3.00 | 3.00 |
| Dimethyl Capramide | 1.00 | 1.00 | 1.00 | 1.00 | 1.00 | 1.00 | 1.00 | 1.00 | 1.00 |
| Isohexadecane | 3.00 | 3.00 | 3.00 | 3.00 | 3.00 | 3.00 | 3.00 | 3.00 | 3.00 |
| Cyclopentasiloxane | 4.00 | 4.00 | 4.00 | 4.00 | 4.00 | 4.00 | 4.00 | 4.00 | 4.00 |
| Stearic Acid | 0.50 | 0.50 | 0.50 | 0.50 | 0.50 | 0.50 | 0.50 | 0.50 | 0.50 |
| PEG-100 Stearate (and) Glyceryl | 0.80 | 0.80 | 0.80 | 0.80 | 0.80 | 0.80 | 0.80 | 0.80 | 0.80 |
| Stearate | | | | | | | | | |
| Potassium Cetyl Phosphate | 1.30 | 1.30 | 1.30 | 1.30 | 1.30 | 1.30 | 1.30 | 1.30 | 1.30 |
| PVP/Eicosene Copolymer | 1.00 | 1.00 | 1.00 | 1.00 | 1.00 | 1.00 | 1.00 | 1.00 | 1.00 |
| Bis-Ethylhexyloxyphenol Methoxyphenyl | 0.00 | 2.50 | 5.00 | 10.00 | 0.00 | 2.50 | 5.00 | 10.00 | 0.00 |
| Triazine (Tinosorb S) | | | | | | | | | |
| Butyl Methoxydibenzoylmethane | 0.00 | 5.00 | 2.50 | 0.00 | 5.00 | 2.50 | 0.00 | 5.00 | 2.50 |
| Methylene Bis-Benzotriazolyl | 10.00 | 5.00 | 0.00 | 10.00 | 5.00 | 0.00 | 10.00 | 5.00 | 0.00 |
| Tetramethylbutylphenol (and) | | | | | | | | | |
| water (and) decyl glucoside | | | | | | | | | |
| (and) propylene glycol (and) | | | | | | | | | |
| xanthan gum (Tinosorb M) | | | | | | | | | |
| Water | Qs to 100 | Qs to 100 | Qs to 100 | Qs to 100 | Qs to 100 | Qs to 100 | Qs to 100 | Qs to 100 | Qs to 100 |
| Propylene Glycol | 5.00 | 5.00 | 5.00 | 5.00 | 5.00 | 5.00 | 5.00 | 5.00 | 5.00 |
| Xanthan Gum | 0.20 | 0.20 | 0.20 | 0.20 | 0.20 | 0.20 | 0.20 | 0.20 | 0.20 |
| Acrylates/C10-30 Alkyl Acrylate | 0.15 | 0.15 | 0.15 | 0.15 | 0.15 | 0.15 | 0.15 | 0.15 | 0.15 |
| Crosspolymer | | | | | | | | | |
| Disodium EDTA | 0.15 | 0.15 | 0.15 | 0.15 | 0.15 | 0.15 | 0.15 | 0.15 | 0.15 |
| Triethanolamine | Qs | Qs | Qs | Qs | Qs | Qs | Qs | Qs | Qs |
| Dimethicone | 0.40 | 0.40 | 0.40 | 0.40 | 0.40 | 0.40 | 0.40 | 0.40 | 0.40 |
| Tocopheryl Acetate | 0.90 | 0.90 | 0.90 | 0.90 | 0.90 | 0.90 | 0.90 | 0.90 | 0.90 |
| pyranopyranone derivative (Table | 0.10 | 0.25 | 0.50 | 0.10 | 0.25 | 0.50 | 0.10 | 0.25 | 0.50 |
| 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Water resistant Sunscreen Emulsion

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Polyglyceryl-10 Pentastearate (and) | 2.50 | 2.50 | 2.50 | 2.50 | 2.50 | 2.50 | 2.50 | 2.50 | 2.50 |
| Behenyl Alcohol (and) Sodium | | | | | | | | | |
| Stearoyl Lactylate | | | | | | | | | |
| Phenethyl Benzoate | 4.00 | 4.00 | 4.00 | 4.00 | 4.00 | 4.00 | 4.00 | 4.00 | 4.00 |
| Triisodecyl Trimellitate | 3.00 | 3.00 | 3.00 | 3.00 | 3.00 | 3.00 | 3.00 | 3.00 | 3.00 |
| Dimethyl Capramide | 1.00 | 1.00 | 1.00 | 1.00 | 1.00 | 1.00 | 1.00 | 1.00 | 1.00 |
| VP/Eicosene Copolymer | 1.50 | 1.50 | 1.50 | 1.50 | 1.50 | 1.50 | 1.50 | 1.50 | 1.50 |
| Stearyl Alcohol | 1.50 | 1.50 | 1.50 | 1.50 | 1.50 | 1.50 | 1.50 | 1.50 | 1.50 |
| Squalane | 2.50 | 2.50 | 2.50 | 2.50 | 2.50 | 2.50 | 2.50 | 2.50 | 2.50 |
| C12-15 Alkyl Benzoate | 2.50 | 2.50 | 2.50 | 2.50 | 2.50 | 2.50 | 2.50 | 2.50 | 2.50 |
| Bis-Ethylhexyloxyphenol Methoxy- | 0.00 | 2.50 | 5.00 | 10.00 | 0.00 | 2.50 | 5.00 | 10.00 | 0.00 |
| phenyl Triazine (Tinosorb S) | | | | | | | | | |
| Diethylamino Hydroxy Benzoyl | 0.00 | 5.00 | 10.00 | 0.00 | 5.00 | 10.00 | 0.00 | 5.00 | 10.00 |
| Hexyl Benzoate | | | | | | | | | |
| Methylene Bis-Benzotriazolyl Tetra- | 10.00 | 5.00 | 0.00 | 10.00 | 5.00 | 0.00 | 10.00 | 5.00 | 0.00 |
| methylbutylphenol (and) water (and) | | | | | | | | | |
| decyl glucoside (and) propylene glycol | | | | | | | | | |
| (and) xanthan gum (Tinosorb M) | | | | | | | | | |
| Water | qs to 100 | qs to 100 | qs to 100 | qs to 100 | qs to 100 | qs to 100 | qs to 100 | qs to 100 | qs to 100 |
| Glycerin | 1.80 | 1.80 | 1.80 | 1.80 | 1.80 | 1.80 | 1.80 | 1.80 | 1.80 |
| Steareth-10 Allyl Ether/Acrylates | 0.80 | 0.80 | 0.80 | 0.80 | 0.80 | 0.80 | 0.80 | 0.80 | 0.80 |
| Copolymer | | | | | | | | | |
| VP/Hexadecene Copolymer | 2.70 | 2.70 | 2.70 | 2.70 | 2.70 | 2.70 | 2.70 | 2.70 | 2.70 |
| Cyclomethicone | 1.50 | 1.50 | 1.50 | 1.50 | 1.50 | 1.50 | 1.50 | 1.50 | 1.50 |
| Water (and) Tocopheryl Acetate | 3.50 | 3.50 | 3.50 | 3.50 | 3.50 | 3.50 | 3.50 | 3.50 | 3.50 |
| (and) Caprylic/Capric Triglyceride | | | | | | | | | |
| (and) Polysorbate 80 (and) Lecithin | | | | | | | | | |
| Fragrance | qs | qs | qs | qs | qs | qs | qs | qs | qs |
| Water (and) Sodium Hydroxide | qs | qs | qs | qs | qs | qs | qs | qs | qs |
| pyranopyranone derivative (Table | 0.10 | 0.25 | 0.50 | 0.10 | 0.25 | 0.50 | 0.10 | 0.25 | 0.50 |
| 1) | | | | | | | | | |
| BIOCIDE/PRESERVATIVE (Table | 0.00 | 0.00 | 0.00 | 0.25 | 0.25 | 0.25 | 0.50 | 0.50 | 0.50 |
| 2) | | | | | | | | | |

| | Every Day Lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Stearyl Phosphate | 5.00 | 5.00 | 5.00 | 5.00 | 5.00 | 5.00 | 5.00 | 5.00 | 5.00 |
| Phenethyl Benzoate | 3.00 | 3.00 | 3.00 | 3.00 | 3.00 | 3.00 | 3.00 | 3.00 | 3.00 |
| Triisodecyl Trimellitate | 3.00 | 3.00 | 3.00 | 3.00 | 3.00 | 3.00 | 3.00 | 3.00 | 3.00 |
| Dimethyl Capramide | 1.00 | 1.00 | 1.00 | 1.00 | 1.00 | 1.00 | 1.00 | 1.00 | 1.00 |
| Tricontanyl PVP | 1.00 | 1.00 | 1.00 | 1.00 | 1.00 | 1.00 | 1.00 | 1.00 | 1.00 |
| Ethoxydiglycol Oleate | 1.50 | 1.50 | 1.50 | 1.50 | 1.50 | 1.50 | 1.50 | 1.50 | 1.50 |
| Squalane | 3.00 | 3.00 | 3.00 | 3.00 | 3.00 | 3.00 | 3.00 | 3.00 | 3.00 |
| C12-15 Alkyl Benzoate | 2.00 | 2.00 | 2.00 | 2.00 | 2.00 | 2.00 | 2.00 | 2.00 | 2.00 |
| Glyceryl Stearate | 2.00 | 2.00 | 2.00 | 2.00 | 2.00 | 2.00 | 2.00 | 2.00 | 2.00 |
| Cetyl Alcohol | 2.00 | 2.00 | 2.00 | 2.00 | 2.00 | 2.00 | 2.00 | 2.00 | 2.00 |
| Bis-Ethylhexyloxyphenol Methoxy- | 0.00 | 2.50 | 5.00 | 10.00 | 0.00 | 2.50 | 5.00 | 10.00 | 0.00 |
| phenyl Triazine (Tinosorb S) | | | | | | | | | |
| Diethylhexyl Butamido Triazone | 0.00 | 5.00 | 10.00 | 0.00 | 5.00 | 10.00 | 0.00 | 5.00 | 10.00 |
| Methylene Bis-Benzotriazolyl | 10.00 | 5.00 | 0.00 | 10.00 | 5.00 | 0.00 | 10.00 | 5.00 | 0.00 |
| Tetramethylbutylphenol (and) | | | | | | | | | |
| water (and) decyl glucoside (and) | | | | | | | | | |
| propylene glycol (and) xanthan | | | | | | | | | |
| gum (Tinosorb M) | | | | | | | | | |
| Water | qs | qs | qs | qs | qs | qs | qs | qs | qs |
| | to | to | to | to | to | to | to | to | to |
| | 100 | 100 | 100 | 100 | 100 | 100 | 100 | 100 | 100 |
| Steareth-10 Allyl Ether/Acrylates | 0.50 | 0.50 | 0.50 | 0.50 | 0.50 | 0.50 | 0.50 | 0.50 | 0.50 |
| Copolymer | | | | | | | | | |
| Glycerin | 2.50 | 2.50 | 2.50 | 2.50 | 2.50 | 2.50 | 2.50 | 2.50 | 2.50 |
| Diazolidinyl Urea (and) Iodopropynyl | 0.15 | 0.15 | 0.15 | 0.15 | 0.15 | 0.15 | 0.15 | 0.15 | 0.15 |
| Butylcarbamate | | | | | | | | | |
| Sodium Lauroyl Glutamate | 0.70 | 0.70 | 0.70 | 0.70 | 0.70 | 0.70 | 0.70 | 0.70 | 0.70 |
| Cyclopentasiloxane (and) | 1.50 | 1.50 | 1.50 | 1.50 | 1.50 | 1.50 | 1.50 | 1.50 | 1.50 |
| Dimethiconol | | | | | | | | | |
| Triethanolamine | 1.85 | 1.85 | 1.85 | 1.85 | 1.85 | 1.85 | 1.85 | 1.85 | 1.85 |
| Citric Acid (and) Silver Citrate | 0.10 | 0.10 | 0.10 | 0.10 | 0.10 | 0.10 | 0.10 | 0.10 | 0.10 |
| Sclerotium Gum | 0.10 | 0.10 | 0.10 | 0.10 | 0.10 | 0.10 | 0.10 | 0.10 | 0.10 |
| pyranopyranone derivative (Table 1) | 0.10 | 0.25 | 0.50 | 0.10 | 0.25 | 0.50 | 0.10 | 0.25 | 0.50 |
| BIOCIDE/PRESERVATIVE (Table 2) | 0.00 | 0.00 | 0.00 | 0.25 | 0.25 | 0.25 | 0.50 | 0.50 | 0.50 |

| | Everyday Moisturizing Cream | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Water | qs. | qs. | qs. | qs. | qs. | qs. | qs. | qs. | qs. |
| | 100 | 100 | 100 | 100 | 100 | 100 | 100 | 100 | 100 |
| Hydroxyethylcellulose | 0.50 | 0.50 | 0.50 | 0.50 | 0.50 | 0.50 | 0.50 | 0.50 | 0.50 |
| Panthenol | 0.50 | 0.50 | 0.50 | 0.50 | 0.50 | 0.50 | 0.50 | 0.50 | 0.50 |
| Preservative | 0.20 | 0.20 | 0.20 | 0.20 | 0.20 | 0.20 | 0.20 | 0.20 | 0.20 |
| Tocopheryl acetate | 0.50 | 0.50 | 0.50 | 0.50 | 0.50 | 0.50 | 0.50 | 0.50 | 0.50 |
| Grape seed oil | 4.00 | 4.00 | 4.00 | 4.00 | 4.00 | 4.00 | 4.00 | 4.00 | 4.00 |
| Almond | 5.00 | 5.00 | 5.00 | 5.00 | 5.00 | 5.00 | 5.00 | 5.00 | 5.00 |
| Caprilyc/Capric triglyceride | 6.00 | 6.00 | 6.00 | 6.00 | 6.00 | 6.00 | 6.00 | 6.00 | 6.00 |
| Caprylyl methicone | 2.00 | 2.00 | 2.00 | 2.00 | 2.00 | 2.00 | 2.00 | 2.00 | 2.00 |
| Dimethicone | 0.50 | 0.50 | 0.50 | 0.50 | 0.50 | 0.50 | 0.50 | 0.50 | 0.50 |
| Glyceryl stearate (and) cetearyl | 5.00 | 5.00 | 5.00 | 5.00 | 5.00 | 5.00 | 5.00 | 5.00 | 5.00 |
| alcohol (and) sodium stearoyl lactylate | | | | | | | | | |
| Evening primrose oil | 1.00 | 1.00 | 1.00 | 1.00 | 1.00 | 1.00 | 1.00 | 1.00 | 1.00 |
| Sodium hyaluronate, 1% aq. soln | 0.50 | 0.50 | 0.50 | 0.50 | 0.50 | 0.50 | 0.50 | 0.50 | 0.50 |
| Hydrocotyl extract | 2.00 | 2.00 | 2.00 | 2.00 | 2.00 | 2.00 | 2.00 | 2.00 | 2.00 |
| pyranopyranone derivative (Table 1) | 0.10 | 0.25 | 0.50 | 0.10 | 0.25 | 0.50 | 0.10 | 0.25 | 0.50 |
| BIOCIDE/PRESERVATIVE (Table 2) | 0.00 | 0.00 | 0.00 | 0.25 | 0.25 | 0.25 | 0.50 | 0.50 | 0.50 |

| | O/W nonionic formula | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Cetyl Alcohol | 1.50 | 1.50 | 1.50 | 1.50 | 1.50 | 1.50 | 1.50 | 1.50 | 1.50 |
| C12-15 Alkyl Benzoate | 5.00 | 5.00 | 5.00 | 5.00 | 5.00 | 5.00 | 5.00 | 5.00 | 5.00 |
| Cetaryl isononoate | 5.00 | 5.00 | 5.00 | 5.00 | 5.00 | 5.00 | 5.00 | 5.00 | 5.00 |
| Ethoxydiglycol Oleate | 2.00 | 2.00 | 2.00 | 2.00 | 2.00 | 2.00 | 2.00 | 2.00 | 2.00 |
| Dioctyl Ether | 3.50 | 3.50 | 3.50 | 3.50 | 3.50 | 3.50 | 3.50 | 3.50 | 3.50 |
| Isohexadecane | 3.00 | 3.00 | 3.00 | 3.00 | 3.00 | 3.00 | 3.00 | 3.00 | 3.00 |
| Ceteareth-15 (and) | 3.00 | 3.00 | 3.00 | 3.00 | 3.00 | 3.00 | 3.00 | 3.00 | 3.00 |
| Glyceryl Sterate | | | | | | | | | |
| Stearyl Alcohol | 1.00 | 1.00 | 1.00 | 1.00 | 1.00 | 1.00 | 1.00 | 1.00 | 1.00 |
| Xanthan Gum | 0.25 | 0.25 | 0.25 | 0.25 | 0.25 | 0.25 | 0.25 | 0.25 | 0.25 |
| Water | qs | qs | qs | qs | qs | qs | qs | qs | qs |
| | to | to | to | to | to | to | to | to | to |
| | 100 | 100 | 100 | 100 | 100 | 100 | 100 | 100 | 100 |
| Pyranopyranone | 0.10 | 0.25 | 0.50 | 0.10 | 0.25 | 0.50 | 0.10 | 0.25 | 0.50 |
| derivative (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

| | O/W Lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Stearic Acid | 4.00 | 4.00 | 4.00 | 4.00 | 4.00 | 4.00 | 4.00 | 4.00 | 4.00 |
| Phenethyl Benzoate | 2.00 | 2.00 | 2.00 | 2.00 | 2.00 | 2.00 | 2.00 | 2.00 | 2.00 |
| Tridecyl Trimellitate | 1.00 | 1.00 | 1.00 | 1.00 | 1.00 | 1.00 | 1.00 | 1.00 | 1.00 |
| Dimethyl Capramide | 0.50 | 0.50 | 0.50 | 0.50 | 0.50 | 0.50 | 0.50 | 0.50 | 0.50 |
| Myristyl Alcohol | 1.50 | 1.50 | 1.50 | 1.50 | 1.50 | 1.50 | 1.50 | 1.50 | 1.50 |
| Cetearyl Alcohol | 0.50 | 0.50 | 0.50 | 0.50 | 0.50 | 0.50 | 0.50 | 0.50 | 0.50 |
| PEG-100 Stearate | 4.00 | 4.00 | 4.00 | 4.00 | 4.00 | 4.00 | 4.00 | 4.00 | 4.00 |
| Kaolin | 0.05 | 0.05 | 0.05 | 0.05 | 0.05 | 0.05 | 0.05 | 0.05 | 0.05 |
| Hydroxyethlycellulose | 0.05 | 0.05 | 0.05 | 0.05 | 0.05 | 0.05 | 0.05 | 0.05 | 0.05 |
| Magnesium Aluminium | 0.20 | 0.20 | 0.20 | 0.20 | 0.20 | 0.20 | 0.20 | 0.20 | 0.20 |
| Silicate | | | | | | | | | |
| Mineral Oil | 6.00 | 6.00 | 6.00 | 6.00 | 6.00 | 6.00 | 6.00 | 6.00 | 6.00 |
| Cyclomethicone | 4.00 | 4.00 | 4.00 | 4.00 | 4.00 | 4.00 | 4.00 | 4.00 | 4.00 |
| Dimethicone | 3.00 | 3.00 | 3.00 | 3.00 | 3.00 | 3.00 | 3.00 | 3.00 | 3.00 |
| PEG-180/Laureth-50/ | 0.50 | 0.50 | 0.50 | 0.50 | 0.50 | 0.50 | 0.50 | 0.50 | 0.50 |
| TMMG Copolymer | | | | | | | | | |
| Glycerin | 3.00 | 3.00 | 3.00 | 3.00 | 3.00 | 3.00 | 3.00 | 3.00 | 3.00 |
| Bis-Ethylhexyloxyphenol | 0.00 | 2.50 | 5.00 | 10.00 | 0.00 | 2.50 | 5.00 | 10.00 | 0.00 |
| Methoxyphenyl Triazine | | | | | | | | | |
| (Tinosorb S) | | | | | | | | | |
| Zinc Oxide | 0.00 | 5.00 | 10.00 | 0.00 | 5.00 | 10.00 | 0.00 | 5.00 | 10.00 |
| Methylene Bis-Benzotriazolyl | 10.00 | 5.00 | 0.00 | 10.00 | 5.00 | 0.00 | 10.00 | 5.00 | 0.00 |
| Tetramethylbutylphenol (and) | | | | | | | | | |
| water (and) decyl glucoside | | | | | | | | | |
| (and) propylene glycol (and) | | | | | | | | | |
| xanthan gum (Tinosorb M) | | | | | | | | | |
| Hydrogenated Coco- | 5.00 | 5.00 | 5.00 | 5.00 | 5.00 | 5.00 | 5.00 | 5.00 | 5.00 |
| Glycerides | | | | | | | | | |
| Parfume | q.s. | q.s. | q.s. | q.s. | q.s. | q.s. | q.s. | q.s. | q.s. |
| Water | ad | ad | ad | ad | ad | ad | ad | ad | ad |
| | 100.00 | 100.00 | 100.00 | 100.00 | 100.00 | 100.00 | 100.00 | 100.00 | 100.00 |
| Propellent | Qs | Qs | Qs | Qs | Qs | Qs | Qs | Qs | Qs |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| pH-value adjusted to 5.0-6.5 | | | | | | | | | |

Solid-stabilizing O/W Emulsions

| | Examples | | | | |
|---|---|---|---|---|---|
| Component [% b.w.] | a | b | c | d | e |
| Mineral Oil | — | — | 16.0 | 16.0 | — |
| Octyldodecanol | 9.0 | 9.0 | 5.0 | — | — |
| Caprylic/Capric.Triglyceride | 9.0 | 9.0 | 6.0 | — | — |
| C12-15- Alkyl Benzoate | — | — | — | 5.0 | 8.0 |
| Butylen Glycol Dicaprylate/Dicaprate | — | — | — | — | 8.0 |
| Dicaprylyl Ether | 9.0 | — | — | 4.0 | — |
| Dicaprylyl Carbonate | — | 9.0 | — | — | — |
| Hydroxyoctacosanyl Hydroxystearate | 2.0 | 2.0 | 2.0 | 2.0 | 1.5 |
| Disteardimonium Hectorit | 1.0 | 0.75 | 0.5 | 0.5 | 0.25 |
| Cera Microcristallina + Paraffinum Liquidum | — | — | — | — | 5.0 |
| Hydroxypropyl Methylcellulose | — | — | — | — | 0.05 |
| Dimethicone | — | — | — | — | 3.0 |
| Ethylhexylmethoxycinnamate | — | — | — | — | 3.0 |
| 4-Methylbenzylidene Camphor | — | — | — | — | 4.0 |
| Diethylhexyl Butamido Triazone | — | — | — | — | 4.0 |
| 2,2′-Methylene-bis-(6-(2H-benzotriazole-2-yl)- | — | — | — | — | 4.0 |
| 4-(1,1,3,3-tetramethylbutyl)-phenol | | | | | |
| (Tinosorb M) | | | | | |
| 2,4-bis{[4-(2-ethylhexyloxy)-2-hydroxy]- | — | 0.5 | — | 2.00 | 1.00 |
| phenyl}-6-(4-methoxyphenyl)-(1,3,5)-triazine | | | | | |
| (Tinosorb S) | | | | | |
| Drometrizol Trisiloxane | — | 0.50 | — | 1.00 | — |
| Terephthaliden Dicamphor Sulfonic Acid | — | 1.00 | 0.50 | — | 1.50 |
| Phenyl Dibenzimidazol Tetrasulfonic Acid | — | — | — | 1.50 | 0.5 |
| Titanium Dioxide + Alumina + Simethicone + | — | 2.0 | 4.0 | 2.0 | 4.0 |
| Aqua | | | | | |
| Titanium Dioxid + Trimethoxycaprylylsilan | — | — | — | — | 3.0 |
| Zinc Oxide | — | — | — | 6.0 | — |
| Silica Dimethyl Silylate | — | — | 1.0 | — | — |
| 2,4,6-Tris(p-biphenylyl)-s-triazine | 1 | 2 | 3 | 4 | 4 |
| Starch/-Sodiummetaphosphate-Polymer | — | 0.5 | — | — | — |
| Tapioca Starch | — | — | — | 1.0 | — |
| Dioic Acid | 0.80 | 0.10 | 0.40 | 0.50 | 1.00 |
| NaCl | 1.0 | 1.0 | 1.0 | 1.0 | 1.0 |
| Glycerine | 5.0 | 10.0 | 3.0 | 6.0 | 10.0 |
| Trisodium EDTA | — | 1.0 | — | 1.0 | — |
| Methylparabene | 0.21 | — | — | — | 0.2 |
| Propylparabene | 0.07 | — | — | — | — |
| Phenoxyethanol | 0.5 | — | 0.4 | 1.0 | 0.5 |
| Hexamidin Diisethionate | — | — | — | — | 0.08 |
| Diazolidinyl Urea | — | — | 0.28 | 0.28 | — |
| Alcohol | — | — | — | 2.5 | — |
| Water | ad 100 | ad 100 | ad 100 | ad 100 | ad 100 |
| Pyranopyranone derivative (Table 1) | 0.10 | 0.20 | 0.30 | 0.40 | 0.50 |
| BIOCIDE/PRESERVATIVE (Table 2) | 0.40 | 0.30 | 0.20 | 0.10 | 0.50 |
| Vitamins A, C or E (free or derivative forms) | 0.80 | 0.50 | — | 1.00 | — |
| Fragrance | qs | qs | qs | qs | qs |

Pickering O/W Emulsions

| | Examples | | | |
|---|---|---|---|---|
| Component [% b.w.] | a | b | c | d |
| Octyldodecanol | 5.50 | 5.50 | 5.00 | 5.50 |
| C12-15- Alkyl Benzoate | 6.50 | 6.50 | 6.00 | 6.50 |
| Butylene Glycol Caprylate/Caprate | — | — | 4.50 | — |
| Dicaprylyl Ether | 5.50 | 5.50 | 3.50 | 5.50 |
| Hydroxyoctacosanyl Hydroxystearate | 2.00 | 2.00 | 2.50 | 2.00 |
| Disteardimonium Hectorite | 1.00 | 1.00 | 0.50 | 1.00 |
| Butyl Methoxydibenzoylmethane | — | — | 1.00 | — |
| Methylbenzylidene Camphor | — | — | 2.00 | — |
| Ethylhexyltriazone | — | — | 1.00 | — |
| 2.2′-Methylene-bis-(6-(2H-benzotriazole-2-yl)-4- | 2.00 | 1.00 | — | 4.00 |
| (1.1.3.3-tetramethylbutyl)-phenol (Tinosorb M) | | | | |
| Titanium Dioxide + Alumina + Simethicone + Aqua | — | — | 0.50 | — |
| Titanium Dioxide + Trimethoxycaprylylsilane | 4.00 | 4.00 | 2.00 | 4.00 |
| Zinc Oxide | 2.00 | 2.00 | — | 2.00 |
| Bariumsulfate | 0.50 | 0.50 | — | 0.50 |
| Boron Nitride | 2.00 | 2.00 | 1.50 | 2.00 |
| NaCl | 1.00 | 1.00 | — | 1.00 |
| MgSO₄ | — | — | 0.70 | — |
| DHA | — | — | 5.00 | — |
| Glycerine | 10.00 | 10.00- | 5.00 | 10.00 |
| Trisodium EDTA | 1.00 | 1.00 | 1.00 | 1.00 |
| Porpylene Carbonate | 0.33 | 0.33 | — | 0.33 |
| Repellent 3535 | — | — | 2.00 | — |
| Hexamidine Diisethionate | 0.08 | 0.08 | 0.08 | 0.08 |
| Water | Qsp 100 | Qsp 100 | Qsp 100 | Qsp 100 |
| Pyranopyranone derivative (Table 1) | 0.10 | 0.20 | 0.30 | 0.40 |
| BIOCIDE/PRESERVATIVE (Table 2) | 0.40 | 0.30 | 0.20 | 0.10 |
| Vitamins A, C or E (free or derivative forms) | 0.80 | 0.50 | — | 1.00 |
| Fragrance | qs | qs | qs | qs |

| | Emulsifier free lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Ceteareth-20 | 1.00 | 1.00 | 1.00 | 1.00 | 1.00 | 1.00 | 1.00 | 1.00 | 1.00 |
| Xanthan Gum | 0.50 | 0.50 | 0.50 | 0.50 | 0.50 | 0.50 | 0.50 | 0.50 | 0.50 |
| Bis-Ethylhexyloxyphenol | 0.00 | 2.50 | 5.00 | 10.00 | 0.00 | 2.50 | 5.00 | 10.00 | 0.00 |
| Methoxyphenyl Triazine | | | | | | | | | |
| (Tinosorb S) | | | | | | | | | |
| Homosalate | 0.00 | 5.00 | 10.00 | 0.00 | 5.00 | 10.00 | 0.00 | 5.00 | 10.00 |
| Methylene Bis- | 10.00 | 5.00 | 0.00 | 10.00 | 5.00 | 0.00 | 10.00 | 5.00 | 0.00 |
| Benzotriazolyl Tetramethyl- | | | | | | | | | |
| butylphenol (and) water | | | | | | | | | |
| (and) decyl glucoside (and) | | | | | | | | | |
| propylene glycol (and) | | | | | | | | | |
| xanthan gum (Tinosorb M) | | | | | | | | | |
| Phenethyl Benzoate | 4.00 | 4.00 | 4.00 | 4.00 | 4.00 | 4.00 | 4.00 | 4.00 | 4.00 |
| Triisodecyl Trimellitate | 2.00 | 2.00 | 2.00 | 2.00 | 2.00 | 2.00 | 2.00 | 2.00 | 2.00 |
| Tridecyl Trimellitate | 1.00 | 1.00 | 1.00 | 1.00 | 1.00 | 1.00 | 1.00 | 1.00 | 1.00 |
| Dimethyl Capramide | 1.00 | 1.00 | 1.00 | 1.00 | 1.00 | 1.00 | 1.00 | 1.00 | 1.00 |
| C12-15 Alkyl Benzoate | 1.00 | 1.00 | 1.00 | 1.00 | 1.00 | 1.00 | 1.00 | 1.00 | 1.00 |
| Butylenglycol Dicapry- | 2.00 | 2.00 | 2.00 | 2.00 | 2.00 | 2.00 | 2.00 | 2.00 | 2.00 |
| lat/Dicaprate | | | | | | | | | |
| Cyclomethicone | 3.00 | 3.00 | 3.00 | 3.00 | 3.00 | 3.00 | 3.00 | 3.00 | 3.00 |
| PVP Hexadecene | 0.50 | 0.50 | 0.50 | 0.50 | 0.50 | 0.50 | 0.50 | 0.50 | 0.50 |
| Copolymer | | | | | | | | | |
| Glycerin | 3.00 | 3.00 | 3.00 | 3.00 | 3.00 | 3.00 | 3.00 | 3.00 | 3.00 |
| Vitamin E Acetate | 0.50 | 0.50 | 0.50 | 0.50 | 0.50 | 0.50 | 0.50 | 0.50 | 0.50 |
| Polyethylene Terephthalate | 0.40 | 0.40 | 0.40 | 0.40 | 0.40 | 0.40 | 0.40 | 0.40 | 0.40 |
| Ethanol | 3.00 | 3.00 | 3.00 | 3.00 | 3.00 | 3.00 | 3.00 | 3.00 | 3.00 |
| Parfume | 0.20 | 0.20 | 0.20 | 0.20 | 0.20 | 0.20 | 0.20 | 0.20 | 0.20 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Water | ad. | ad. | ad. | ad. | ad. | ad. | ad. | ad. | ad. |
| | 100 | 100 | 100 | 100 | 100 | 100 | 100 | 100 | 100 |

| | Emulsifier free face care | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Sodium Carbomer | 0.20 | 0.20 | 0.20 | 0.20 | 0.20 | 0.20 | 0.20 | 0.20 | 0.20 |
| Xanthan Gum | 0.30 | 0.30 | 0.30 | 0.30 | 0.30 | 0.30 | 0.30 | 0.30 | 0.30 |
| Bis-Ethylhexyloxyphenol | 0.00 | 2.50 | 5.00 | 10.00 | 0.00 | 2.50 | 5.00 | 10.00 | 0.00 |
| Methoxyphenyl Triazine | | | | | | | | | |
| (Tinosorb S) | | | | | | | | | |
| Isoamyl p-Methoxycinnamate | 0.00 | 5.00 | 10.00 | 0.00 | 5.00 | 10.00 | 0.00 | 5.00 | 10.00 |
| Methylene Bis-Benzotriazolyl | 10.00 | 5.00 | 0.00 | 10.00 | 5.00 | 0.00 | 10.00 | 5.00 | 0.00 |
| Tetramethylbutylphenol (and) | | | | | | | | | |
| water (and) decyl glucoside | | | | | | | | | |
| (and) propylene glycol (and) | | | | | | | | | |
| xanthan gum (Tinosorb M) | | | | | | | | | |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| C12-15 Alkyl Benzoate | 1.00 | 1.00 | 1.00 | 1.00 | 1.00 | 1.00 | 1.00 | 1.00 | 1.00 |
| Dicaprylyl Carbonate | 1.00 | 1.00 | 1.00 | 1.00 | 1.00 | 1.00 | 1.00 | 1.00 | 1.00 |
| Dimethicone | 0.50 | 0.50 | 0.50 | 0.50 | 0.50 | 0.50 | 0.50 | 0.50 | 0.50 |
| Shea Butter | 2.00 | 2.00 | 2.00 | 2.00 | 2.00 | 2.00 | 2.00 | 2.00 | 2.00 |
| Ethylhexylglycerine | 0.50 | 0.50 | 0.50 | 0.50 | 0.50 | 0.50 | 0.50 | 0.50 | 0.50 |
| Glycerin | 7.50 | 7.50 | 7.50 | 7.50 | 7.50 | 7.50 | 7.50 | 7.50 | 7.50 |
| Glycinc Soja | 1.50 | 1.50 | 1.50 | 1.50 | 1.50 | 1.50 | 1.50 | 1.50 | 1.50 |
| Vitamin E Acetate | 0.20 | 0.20 | 0.20 | 0.20 | 0.20 | 0.20 | 0.20 | 0.20 | 0.20 |
| -Glycosil Rutin | 0.30 | 0.30 | 0.30 | 0.30 | 0.30 | 0.30 | 0.30 | 0.30 | 0.30 |
| Polyethylene Terephthalate | 0.50 | 0.50 | 0.50 | 0.50 | 0.50 | 0.50 | 0.50 | 0.50 | 0.50 |
| Trisodium EDTA | 0.30 | 0.30 | 0.30 | 0.30 | 0.30 | 0.30 | 0.30 | 0.30 | 0.30 |
| Ethanol | 7.00 | 7.00 | 7.00 | 7.00 | 7.00 | 7.00 | 7.00 | 7.00 | 7.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Water | ad. 100 | ad. 100 | ad. 100 | ad. 100 | ad. 100 | ad. 100 | ad. 100 | ad. 100 | ad. 100 |

| | Skin care spray | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Butylene Glycol Dicapry- | 6.00 | 6.00 | 6.00 | 6.00 | 6.00 | 6.00 | 6.00 | 6.00 | 6.00 |
| late/Dicaprate | | | | | | | | | |
| C18-36 Acid Triglyceride | 0.70 | 0.70 | 0.70 | 0.70 | 0.70 | 0.70 | 0.70 | 0.70 | 0.70 |
| Ceteareth-20 | 0.60 | 0.60 | 0.60 | 0.60 | 0.60 | 0.60 | 0.60 | 0.60 | 0.60 |
| Phenethyl Benzoate | 4.00 | 4.00 | 4.00 | 4.00 | 4.00 | 4.00 | 4.00 | 4.00 | 4.00 |
| Triisodecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| C12-15 Alkyl Benzoate | 6.00 | 6.00 | 6.00 | 6.00 | 6.00 | 6.00 | 6.00 | 6.00 | 6.00 |
| Taurine | 0.40 | 0.40 | 0.40 | 0.40 | 0.40 | 0.40 | 0.40 | 0.40 | 0.40 |
| PVP/Hexadecene Copolymer | 0.50 | 0.50 | 0.50 | 0.50 | 0.50 | 0.50 | 0.50 | 0.50 | 0.50 |
| Bis-Ethylhexyloxyphenol Methoxy- | 0.00 | 2.50 | 5.00 | 10.00 | 0.00 | 2.50 | 5.00 | 10.00 | 0.00 |
| phenyl Triazine (Tinosorb S) | | | | | | | | | |
| Polysilicone-15 | 0.00 | 5.00 | 10.00 | 0.00 | 5.00 | 10.00 | 0.00 | 5.00 | 10.00 |
| Methylene Bis-Benzotriazolyl Tetra- | 10.00 | 5.00 | 0.00 | 10.00 | 5.00 | 0.00 | 10.00 | 5.00 | 0.00 |
| methylbutylphenol (and) water (and) | | | | | | | | | |
| decyl glucoside (and) propylene gly- | | | | | | | | | |
| col (and) xanthan gum (Tinosorb M) | | | | | | | | | |
| Water | qs. | qs. | qs. | qs. | qs. | qs. | qs. | qs. | qs. |
| | To | To | To | To | To | To | To | To | To |
| | 100 | 100 | 100 | 100 | 100 | 100 | 100 | 100 | 100 |
| Glycerin | 6.00 | 6.00 | 6.00 | 6.00 | 6.00 | 6.00 | 6.00 | 6.00 | 6.00 |
| Methylpropanediol | 1.00 | 1.00 | 1.00 | 1.00 | 1.00 | 1.00 | 1.00 | 1.00 | 1.00 |
| Acrylates/C10-30 Alkyl Acrylate | 0.18 | 0.18 | 0.18 | 0.18 | 0.18 | 0.18 | 0.18 | 0.18 | 0.18 |
| Crosspolymer | | | | | | | | | |
| Trisodium EDTA | 0.10 | 0.10 | 0.10 | 0.10 | 0.10 | 0.10 | 0.10 | 0.10 | 0.10 |
| Alcohol Denatured | 4.00 | 4.00 | 4.00 | 4.00 | 4.00 | 4.00 | 4.00 | 4.00 | 4.00 |
| Tocopheryl Acetate | 0.50 | 0.50 | 0.50 | 0.50 | 0.50 | 0.50 | 0.50 | 0.50 | 0.50 |
| Pyranopyranone derivative (Table 1) | 0.10 | 0.25 | 0.50 | 0.10 | 0.25 | 0.50 | 0.10 | 0.25 | 0.50 |
| BIOCIDE/PRESERVATIVE (Table 2) | 0.00 | 0.00 | 0.00 | 0.25 | 0.25 | 0.25 | 0.50 | 0.50 | 0.50 |

COLD PROCESS SPRAY

| | Ex. 1 | Ex. 2 | Ex. 3 | Ex. 4 |
|---|---|---|---|---|
| Atlas 2330/Sorbitan-30 (Uniqema) | 3.00 | 3.00 | 3.00 | 3.00 |
| Prisorine 3515/Isostearyl Alcohol | 6.00 | 6.00 | 6.00 | 6.00 |
| (Uniqema) | | | | |
| Sympatens W/4500/Sorbitan | 2.00 | 2.00 | 2.00 | 2.00 |
| Oleate + Polyglycerol | | | | |
| 3-Polyricinoleate (Kolb) | | | | |
| Tween 20/Polysorbate-20 | 1.00 | 1.00 | 1.00 | 1.00 |
| (Uniqema) | | | | |
| Dow Corning 200 (200 cps)/ | 1.00 | 1.00 | 1.00 | 1.00 |
| Dimethicone (Dow Corning) | | | | |
| Glydant Plus Liquid/DMDM | 1.00 | 1.00 | 1.00 | 1.00 |
| Hydantoin and 3-Iodo-2-propynyl | | | | |
| butylcarbamate (Lonza) | | | | |
| Deionized Water/Water (Aqua) | Qsp 100 | Qsp 100 | Qsp 100 | Qsp 100 |
| STRUCTURE XL/Hydroxypropyl | 4.00 | 4.00 | 4.00 | 4.00 |
| Starch Phosphate (National | | | | |
| Starch) | | | | |
| DERMACRYL AQF (45% soln.)/ | 3.35 | 3.35 | 3.35 | 3.35 |
| Acrylates Co-polymer (National | | | | |
| Starch) | | | | |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Sclerotium Gum | 0.50 | 2.50 | 5.00 | 10.00 |

SPRAY EMULSION WATER-RESISTANT

| | Ex. 1 | Ex. 2 | Ex. 3 | Ex. 4 |
|---|---|---|---|---|
| | Wt. % | | | |
| Deionized Water/Water (Aqua) | Qsp 100 | Qsp 100 | Qsp 100 | Qsp 100 |
| Carbopol ETD 2020/(Acrylates/ | 0.18 | 0.18 | 0.18 | 0.18 |
| C10-30 Alkyl Acrylate | | | | |
| Crosspolymer) (Noveon) | | | | |
| Versene 100/Tetrasodium EDTA | 0.15 | 0.15 | 0.15 | 0.15 |
| (Dow Chemical) | | | | |
| Phenonip/Phenoxyethanol, | 0.80 | 0.80 | 0.80 | 0.80 |
| Methylparaben, Butylparaben, | | | | |
| Ethylparaben, Propylparaben | | | | |
| (Nipa) | | | | |
| 1,4-Butanediol/Butylene Glycol | 4.00 | 4.00 | 4.00 | 4.00 |
| (BASF) | | | | |
| DERMACRYL 79/(Acrylates/ | 2.00 | 2.00 | 2.00 | 2.00 |
| Octylacrylamide Copolymer) | | | | |
| (National Starch) | | | | |
| Triethanolamine 99%/ | 0.71 | 0.71 | 0.71 | 0.71 |
| Triethanolamine | | | | |
| Tego Care 450/Polyglyceryl-3 | 3.00 | 3.00 | 3.00 | 3.00 |
| Methylglucose Distearate | | | | |
| (Goldschmidt) | | | | |
| Synchrowax ERL-C/C18-36 Acid | 0.60 | 0.60 | 0.60 | 0.60 |
| Glycol Ester (Croda) | | | | |
| Dow Corning 200/100 cst/ | 3.00 | 3.00 | 3.00 | 3.00 |
| Dimethicone (Dow Corning) | | | | |
| Vitamin E Acetate/Tocopheryl | 0.10 | 0.10 | 0.10 | 0.10 |
| Acetate (Roche Vitamin) | | | | |
| Fragrance/Fragrance (Perfume) | q.s. | q.s. | q.s. | q.s. |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Sclerotium Gum | 0.50 | 2.50 | 5.00 | 10.00 |

O/W Spray without Emulsifier

| Ingredient | a | b | c | d |
|---|---|---|---|---|
| TEGO ® Carbomer 341 ER | 0.3 | 0.3 | 0.3 | 0.3 |
| (Acrylates/C10-30 Alkyl Acrylates | | | | |
| Crosspolymer (proposed)) | | | | |
| ABIL ® Wax 9801 (Cetyl | 1.0 | 1.0 | 1.0 | 1.0 |
| Dimethicone) | | | | |
| ABIL ® B 8863 (PEG/ | 1.0 | 1.0 | 1.0 | 1.0 |
| PPG-20/20 Dimethicone) | | | | |
| TEGOSOFT ® CR (Cetyl | 1.0 | 1.0 | 1.0 | 1.0 |
| Ricinoleate) | | | | |
| Dicaprylyl Ether | 5.5 | 5.5 | 5.5 | 5.5 |
| Tocopheryl Acetate | 0.5 | 0.5 | 0.5 | 0.5 |
| Alcohol | 12.0 | 12.0 | 12.0 | 12.0 |
| D-Panthenol (Panthenol, | 1.0 | 1.0 | 1.0 | 1.0 |
| Roche-Vitamins) | | | | |
| EDTA | 0.1 | 0.1 | 0.1 | 0.1 |
| Glycerin | 5.0 | 5.0 | 5.0 | 5.0 |
| Water | Qsp 100 | Qsp 100 | Qsp 100 | Qsp 100 |
| Sodium Hydroxide (10% in water) | 0.4 | 0.4 | 0.4 | 0.4 |
| Scleroglucan Gum | 0.50% | 2.50% | 5.00% | 10.00% |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Vitamins A, C or E (free or | 0.80 | 0.50 | — | 1.00 |
| derivative forms) | | | | |
| Fragrance | qs | qs | qs | qs |

Hydrodispersions

| | Examples | | | | |
|---|---|---|---|---|---|
| Component [% b.w.] | a | b | c | d | e |
| Polyoxyethylene(20)- | 1.00 | — | — | 0.5 | — |
| cetylstearylether | | | | | |
| Cetylalcohol | — | — | 1.00 | — | — |
| Natriumpolyacrylate | — | 0.20 | — | 0.30 | — |
| Acrylate/C10-30-Alkyl- | 0.50 | | 0.40 | 0.10 | 0.10 |
| Acrylate Crosspolymer | | | | | |
| Xanthan Gum | — | 0.30 | 0.15 | — | 0.50 |
| 2-Ethylhexyl | — | — | — | 5.00 | 8.00 |
| Methoxycinnamate | | | | | |
| 2,4-bis{[4-(2-ethyl- | — | 1.50 | 2.00 | — | 2.50 |
| hexyloxy)-2-hydroxy]- | | | | | |
| phenyl}-6-(4-methoxy- | | | | | |
| phenyl)-(1,3,5)-triazine | | | | | |
| (Tinosorb S) | | | | | |
| Dimethicodiethyl- | — | 3.50 | — | — | — |
| benzalmalonate | | | | | |
| Butyl-dimethoxy- | 1.00 | — | 2.00 | — | — |
| dibenzoylmethane | | | | | |
| Diethylhexyl | — | 2.00 | — | 2.00 | 1.00 |
| Butamidotriazone | | | | | |
| Ethylhexyl Tdazone | 4.00 | — | 3.00 | 4.00 | — |
| 4-Methylbenzylidene | — | — | — | — | 2.00 |
| Camphor | | | | | |
| Methylene bis-benzo- | 2.00 | 1.40 | 0.50 | 4.60 | 5.20 |
| triazolyl tetramethyl- | | | | | |
| butylphenol | | | | | |
| (TinosorbM) | | | | | |
| Octocrylene | — | 4.00 | 4.00 | — | 2.50 |
| Phenylen-1,4-bis- | 1.00 | — | 0.50 | — | 2.00 |
| (monosodium-2- | | | | | |
| benzimidazyl-5,7- | | | | | |
| disulfonic Acid) | | | | | |
| Phenylbenzimidazol | 0.50 | — | — | 3.00 | — |
| Sulfonic Acid | | | | | |
| Titanium Dioxide | 0.50 | — | 2.00 | 3.00 | 1.00 |
| Zinc Oxide | 0.50 | 1.00 | 3.00 | — | 2.00 |
| C12-15 Alkyl Benzoate | 2.00 | 2.50 | — | — | — |
| Diethylhexyl-2.6- | 4.00 | — | — | — | — |
| naphthalate | | | | | |
| Dicaprylylether | — | 4.00 | — | — | — |
| Butylenglycol- | 4.00 | — | 2.00 | 6.00 | — |
| Dicaprylate/-Dicaprate | | | | | |
| Dicaprylylcarbonate | — | 2.00 | 6.00 | — | — |
| Dimethicone | — | 0.50 | 1.00 | — | — |
| Polydimethylsiloxane | | | | | |
| Phenylmethyl- | 2.00 | — | — | 0.50 | 2.00 |
| polysiloxane | | | | | |
| Shea Butter | — | 2.00 | — | — | — |
| PVP Hexadecene- | 0.50 | — | — | 0.50 | 1.00 |
| copolymer | | | | | |
| Octoxyglycerine | — | — | 1.00 | — | 0.50 |
| Glycerine | 3.00 | 7.50 | — | 7.50 | 2.50 |
| Glycinesoja | — | — | 1.50 | — | — |
| Dioic Acid | 0.3 | 0.10 | 0.50 | 1.00 | 0.20 |
| DMDM Hydantoine | — | 0.25 | — | 0.60 | 0.45 |
| Parabene | 0.15 | — | 0.50 | 0.30 | — |
| Trisodium EDTA | — | — | 0.70 | — | 1.00 |
| Ethanol | 3.00 | 2.00 | 1.50 | — | 1.00 |
| Water | Ad 100 | Ad 100 | Ad 100 | Ad 100 | ad 100 |
| Pyranopyranone | 0.10 | 0.20 | 0.30 | 0.40 | 0.50 |
| derivative (Table 1) | | | | | |
| BIOCIDE/ | 0.40 | 0.30 | 0.20 | 0.10 | 0.50 |
| PRESERVATIVE | | | | | |
| (Table 2) | | | | | |
| Vitamins A, C or E | 0.80 | 0.50 | — | 1.00 | — |
| (free or as derivative) | | | | | |
| Fragrance | qs | qs | qs | qs | qs |

| | Gel | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Alcohol Denatured | Qs to | Qs to | Qs to | Qs to | Qs to | Qs to | Qs to | Qs to | Qs to |
| | 100 | 100 | 100 | 100 | 100 | 100 | 100 | 100 | 100 |
| Phenethyl Benzoate | 2.00 | 2.00 | 2.00 | 2.00 | 2.00 | 2.00 | 2.00 | 2.00 | 2.00 |
| Triisodecyl Trimellitate | 1.00 | 1.00 | 1.00 | 1.00 | 1.00 | 1.00 | 1.00 | 1.00 | 1.00 |
| Dimethyl Capramide | 0.50 | 0.50 | 0.50 | 0.50 | 0.50 | 0.50 | 0.50 | 0.50 | 0.50 |
| Hydroxypropyl Cellulose | 1.00 | 1.00 | 1.00 | 1.00 | 1.00 | 1.00 | 1.00 | 1.00 | 1.00 |
| Acrylates/Octylacrylamide | 1.00 | 1.00 | 1.00 | 1.00 | 1.00 | 1.00 | 1.00 | 1.00 | 1.00 |
| Copolymer | | | | | | | | | |
| C12-15 Alkyl Benzoate | 10.00 | 10.00 | 10.00 | 10.00 | 10.00 | 10.00 | 10.00 | 10.00 | 10.00 |
| Cyclotetrasiloxane (and) | 3.00 | 3.00 | 3.00 | 3.00 | 3.00 | 3.00 | 3.00 | 3.00 | 3.00 |
| Cyclopentasiloxane | | | | | | | | | |
| PEG/PPG-4/12 Dimethi- | 1.00 | 1.00 | 1.00 | 1.00 | 1.00 | 1.00 | 1.00 | 1.00 | 1.00 |
| cone | | | | | | | | | |
| Bis-Ethylhexyloxyphenol | 0.00 | 2.50 | 5.00 | 10.00 | 0.00 | 2.50 | 5.00 | 10.00 | 0.00 |
| Methoxyphenyl Triazine | | | | | | | | | |
| (Tinosorb S) | | | | | | | | | |
| Ethylhexyl Salicylate | 0.00 | 5.00 | 2.50 | 0.00 | 5.00 | 2.50 | 0.00 | 5.00 | 2.50 |
| Methylene Bis-Benzotria- | 10.00 | 5.00 | 0.00 | 10.00 | 5.00 | 0.00 | 10.00 | 5.00 | 0.00 |
| zolyl Tetramethylbutylphe- | | | | | | | | | |
| nol (and) water (and) decyl | | | | | | | | | |
| glucoside (and) propylene | | | | | | | | | |
| glycol (and) xanthan gum | | | | | | | | | |
| (Tinosorb M) | | | | | | | | | |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

| | Clear Gel | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| SD Alcohol 40 | 44.07 | 44.07 | 44.07 | 44.07 | 44.07 | 44.07 | 44.07 | 44.07 | 44.07 |
| Phenethyl Benzoate | 2.00 | 2.00 | 2.00 | 2.00 | 2.00 | 2.00 | 2.00 | 2.00 | 2.00 |
| Tridecyl Trimellitate | 1.00 | 1.00 | 1.00 | 1.00 | 1.00 | 1.00 | 1.00 | 1.00 | 1.00 |
| Dimethyl Capramide | 0.50 | 0.50 | 0.50 | 0.50 | 0.50 | 0.50 | 0.50 | 0.50 | 0.50 |
| Hydroxypropyl Cellulose | 1.67 | 1.67 | 1.67 | 1.67 | 1.67 | 1.67 | 1.67 | 1.67 | 1.67 |
| Bis-Ethylhexyloxyphenol | 0.00 | 2.50 | 5.00 | 10.00 | 0.00 | 2.50 | 5.00 | 10.00 | 0.00 |
| Methoxyphenyl Triazine | | | | | | | | | |
| (Tinosorb S) | | | | | | | | | |
| Methylene Bis- | 0.00 | 2.00 | 4.00 | 2.00 | 4.00 | 2.00 | 4.00 | 2.00 | 4.00 |
| Benzotriazolyl Tetrameth- | | | | | | | | | |
| ylbutylphenol (and) water | | | | | | | | | |
| (and) decyl glucoside | | | | | | | | | |
| (and) propylene glycol | | | | | | | | | |
| (and) xanthan gum | | | | | | | | | |
| (Tinosorb M) | | | | | | | | | |
| Tris-Biphenyl Triazine | 10.00 | 5.00 | 0.00 | 10.00 | 5.00 | 0.00 | 10.00 | 5.00 | 0.00 |
| Isopropyl PPG-2- | 9.64 | 9.64 | 9.64 | 9.64 | 9.64 | 9.64 | 9.64 | 9.64 | 9.64 |
| Isodeceth-7 Carboxylate | | | | | | | | | |
| SD Alcohol 40 | 24.30 | 24.30 | 24.30 | 24.30 | 24.30 | 24.30 | 24.30 | 24.30 | 24.30 |
| Fragrance | 0.60 | 0.60 | 0.60 | 0.60 | 0.60 | 0.60 | 0.60 | 0.60 | 0.60 |
| Water | Qsp | Qsp | Qsp | Qsp | Qsp | Qsp | Qsp | Qsp | Qsp |
| | 100 | 100 | 100 | 100 | 100 | 100 | 100 | 100 | 100 |
| Sclerotium Gum | 0.10 | 0.10 | 0.10 | 0.10 | 0.10 | 0.10 | 0.10 | 0.10 | 0.10 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Waterproofed Gel

| | Ex. 1 | Ex. 2 | Ex. 3 | Ex. 4 |
|---|---|---|---|---|
| | Wt. % | | | |
| Anhydrous Ethanol | Qsp 100 | Qsp 100 | Qsp 100 | Qsp 100 |
| Hydroxypropyl Cellulose/Klucel | 1.00 | 1.00 | 1.00 | 1.00 |
| MF | | | | |
| Acrylates/Octylacrylamide | 1.00 | 1.00 | 1.00 | 1.00 |
| Copolymer/Dermacryl-79 | | | | |
| C12-15 Alkyl Benzoate/Finsolv | 10.00 | 10.00 | 10.00 | 10.00 |
| TN | | | | |
| Cyclomethicone/Dow Corning 344 | 3.00 | 3.00 | 3.00 | 3.00 |
| Fluid | | | | |
| PEG/PPG-4/12 Dimethicone/ | 1.00 | 1.00 | 1.00 | 1.00 |
| Abil B8852 | | | | |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Sclerotium Gum | 0.50 | 2.50 | 5.00 | 10.00 |

Nourishing Gel

| | Ex. 1 | Ex. 2 | Ex. 3 | Ex. 4 |
|---|---|---|---|---|
| Ingredient | Wt. % | | | |
| Acritamer 940 (Carbomer 940)/ | 1.00 | 1.00 | 1.00 | 1.00 |
| Gelling Agent | | | | |
| Deionized Water | Qsp 100 | Qsp 100 | Qsp 100 | Qsp 100 |
| Ritapan D (d-Panthenol)/ | 0.20 | 0.20 | 0.20 | 0.20 |
| Conditioner | | | | |
| SD Alcohol (Ethanol @ 95%)/ | 10.00 | 10.00 | 10.00 | 10.00 |
| Solvent, Solubilizer | | | | |
| Ritachol (Mineral Oil and Lanolin | 8.00 | 8.00 | 8.00 | 8.00 |
| Alcohol)/Emollient | | | | |
| Pationic ISL (Sodium Isostearoyl | 0.50 | 0.50 | 0.50 | 0.50 |
| Lactylate)/Solubilizer | | | | |
| Fragrance/Odor | q.s. | q.s. | q.s. | q.s. |
| NaOH (18% Soln.)/Neutralizer | 2.40 | 2.40 | 2.40 | 2.40 |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Sclerotium Gum | 0.50 | 2.50 | 5.00 | 10.00 |

| | O/W Foaming Lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Decyl Cocoate | 2.50 | 2.50 | 2.50 | 2.50 | 2.50 | 2.50 | 2.50 | 2.50 | 2.50 |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| Isopropyl Palmitate | 1.60 | 1.60 | 1.60 | 1.60 | 1.60 | 1.60 | 1.60 | 1.60 | 1.60 |
| Tocopheryl Acetate | 0.50 | 0.50 | 0.50 | 0.50 | 0.50 | 0.50 | 0.50 | 0.50 | 0.50 |
| Cetearyl Glucoside | 1.00 | 1.00 | 1.00 | 1.00 | 1.00 | 1.00 | 1.00 | 1.00 | 1.00 |
| Bis-Ethylhexyloxyphenol Methoxy- | 0.00 | 2.50 | 5.00 | 10.0 | 0.00 | 2.50 | 5.00 | 10.00 | 0.00 |
| phenyl Triazine (Tinosorb S) | | | | | | | | | |
| Terephthalylidene Dicamphor Sulfo- | 0.00 | 5.00 | 10.0 | 0.00 | 5.00 | 10.0 | 0.00 | 5.00 | 10.0 |
| nic Acid | | | | | | | | | |
| Methylene Bis-Benzotriazolyl | 10.0 | 5.00 | 0.00 | 10.0 | 5.00 | 0.00 | 10.0 | 5.00 | 0.00 |
| Tetramethylbutylphenol (and) water | | | | | | | | | |
| (and) decyl glucoside (and) propy- | | | | | | | | | |
| lene glycol (and) xanthan gum | | | | | | | | | |
| (Tinosorb M) | | | | | | | | | |
| Glycerin | 2.00 | 2.00 | 2.00 | 2.00 | 2.00 | 2.00 | 2.00 | 2.00 | 2.00 |
| EDTA | 0.10 | 0.10 | 0.10 | 0.10 | 0.10 | 0.10 | 0.10 | 0.10 | 0.10 |
| Water | Qsp | Qsp | Qsp | Qsp | Qsp | Qsp | Qsp | Qsp | Qsp |
| | 100 | 100 | 100 | 100 | 100 | 100 | 100 | 100 | 100 |
| Carbomer | 0.05 | 0.05 | 0.05 | 0.05 | 0.05 | 0.05 | 0.05 | 0.05 | 0.05 |
| Acrylates/C10-30 Alkyl Acrylates | 0.05 | 0.05 | 0.05 | 0.05 | 0.05 | 0.05 | 0.05 | 0.05 | 0.05 |
| Crosspolymer | | | | | | | | | |
| Sodium Hydroxide (10% in water) | q.s. | q.s. | q.s. | q.s. | q.s. | q.s. | q.s. | q.s. | q.s. |
| Pyranopyranone derivative (Table 1) | 0.10 | 0.25 | 0.50 | 0.10 | 0.25 | 0.50 | 0.10 | 0.25 | 0.50 |
| BIOCIDE/PRESERVATIVE (Table 2) | 0.00 | 0.00 | 0.00 | 0.25 | 0.25 | 0.25 | 0.50 | 0.50 | 0.50 |
| Undecylenamidopropyl Betain | 6.70 | 6.70 | 6.70 | 6.70 | 6.70 | 6.70 | 6.70 | 6.70 | 6.70 |
| Sclerotium Gum | 0.10 | 0.10 | 0.10 | 0.10 | 0.10 | 0.10 | 0.10 | 0.10 | 0.10 |

| | Self-tanning Lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Glyceryl stearate (and) | 3.00 | 3.00 | 3.00 | 3.00 | 3.00 | 3.00 | 3.00 | 3.00 | 3.00 |
| ceteth-20 | | | | | | | | | |
| Stearyl alcohol | 1.00 | 1.00 | 1.00 | 1.00 | 1.00 | 1.00 | 1.00 | 1.00 | 1.00 |
| Caprylic/Capric triglyceride | 1.00 | 1.00 | 1.00 | 1.00 | 1.00 | 1.00 | 1.00 | 1.00 | 1.00 |
| Cetearyl ethylhexanoate | 1.00 | 1.00 | 1.00 | 1.00 | 1.00 | 1.00 | 1.00 | 1.00 | 1.00 |
| Diethylhexyl carbonate | 2.00 | 2.00 | 2.00 | 2.00 | 2.00 | 2.00 | 2.00 | 2.00 | 2.00 |
| Glycerin | 2.00 | 2.00 | 2.00 | 2.00 | 2.00 | 2.00 | 2.00 | 2.00 | 2.00 |
| Capryl/capamidopropyl | 7.00 | 7.00 | 7.00 | 7.00 | 7.00 | 7.00 | 7.00 | 7.00 | 7.00 |
| betaine | | | | | | | | | |
| Dihydroxyacetone | 3.50 | 3.50 | 3.50 | 3.50 | 3.50 | 3.50 | 3.50 | 3.50 | 3.50 |
| Erythrulose | 1.50 | 1.50 | 1.50 | 1.50 | 1.50 | 1.50 | 1.50 | 1.50 | 1.50 |
| Water | ad | ad | ad | ad | ad | ad | ad | ad | ad |
| | 100 | 100 | 100 | 100 | 100 | 100 | 100 | 100 | 100 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Crackling Foam UV Protection

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Behenyl Alcohol (and) Glyceryl Stearate | 1.00 | 1.50 | 2.00 | 1.00 | 2.00 | 1.50 | 2.00 | 2.50 | 2.00 |
| (and) Glyceryl Stearate Citrate | | | | | | | | | |
| (and) Sodium Dicocoylethylenediamine | | | | | | | | | |
| PEG-15 Sulfate | | | | | | | | | |
| Dibutyl Adipate | 7.00 | 7.00 | 7.00 | 7.00 | 7.00 | 7.00 | 7.00 | 7.00 | 7.00 |
| Homosalate | 5.00 | 5.00 | 5.00 | 5.00 | 5.00 | 5.00 | 5.00 | 5.00 | 5.00 |
| Ethylhexyl Salicylate | 3.00 | 3.00 | 3.00 | 3.00 | 3.00 | 3.00 | 3.00 | 3.00 | 3.00 |
| Butyl Methoxydibenzoylmethane | 3.00 | 2.00 | 1.00 | 4.00 | 3.00 | 2.00 | 1.00 | 4.00 | 3.00 |
| Bis-Ethylhexyloxyphenol Methoxy- | 2.00 | 3.00 | 3.00 | 2.00 | 2.00 | 3.00 | 3.00 | 2.00 | 3.00 |
| phenyl Triazine | | | | | | | | | |
| Ethylhexyl Triazone | 2.00 | 1.00 | 2.00 | 2.00 | 2.00 | 1.00 | 2.00 | 2.00 | 3.00 |
| Water | qsp | qsp | qsp | qsp | qsp | qsp | qsp | qsp | qsp |
| | 100 | 100 | 100 | 100 | 100 | 100 | 100 | 100 | 100 |
| Disodium EDTA | 0.20 | 0.20 | 0.20 | 0.20 | 0.20 | 0.20 | 0.20 | 0.20 | 0.20 |
| Pentylene Glycol (and) Sclerotium | 2.00 | 2.00 | 2.00 | 2.00 | 2.00 | 2.00 | 2.00 | 2.00 | 2.00 |
| Gum | | | | | | | | | |
| Xanthane gum | 0.20 | 0.20 | 0.20 | 0.20 | 0.20 | 0.20 | 0.20 | 0.20 | 0.20 |
| Sodium Dicocoylethylenediamine | 2.50 | 2.50 | 2.50 | 2.50 | 2.50 | 2.50 | 2.50 | 2.50 | 2.50 |
| PEG-15 Sulfate (and) Sodium | | | | | | | | | |
| Lauroyl Lactylate | | | | | | | | | |
| Cyclopentasiloxane (and) Cyclo- | 3.00 | 3.00 | 3.00 | 3.00 | 3.00 | 3.00 | 3.00 | 3.00 | 3.00 |
| hexasiloxane | | | | | | | | | |
| Alcohol | 3.00 | 3.00 | 3.00 | 3.00 | 3.00 | 3.00 | 3.00 | 3.00 | 3.00 |
| Methylene Bis-Benzotriazolyl | 6.00 | 4.00 | 3.50 | 5.00 | 2.50 | 6.00 | 4.00 | 3.50 | 5.00 |
| Tetramethylbutylphenol (and) Aqua | | | | | | | | | |
| (and) Decyl Glucoside (and) Propylene | | | | | | | | | |
| Glycol (and) Xanthan Gum | | | | | | | | | |

PROPELLANT:

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Butane (N-Butan 1.2 Bar) | 40.00 | 40.00 | 40.00 | 40.00 | 40.00 | 40.00 | 40.00 | 40.00 | 40.00 |
| Dimethyl Ether | 20.00 | 20.00 | 20.00 | 20.00 | 20.00 | 20.00 | 20.00 | 20.00 | 20.00 |
| Pyranopyranone derivative (Table 1) | 0.10 | 0.25 | 0.50 | 0.10 | 0.25 | 0.50 | 0.10 | 0.25 | 0.50 |
| BIOCIDE/PRESERVATIVE (Table 2) | 0.00 | 0.00 | 0.00 | 0.25 | 0.25 | 0.25 | 0.50 | 0.50 | 0.50 |

Continuous Spray UV protection

| | INCI-Name | % w/w (as supplied) | | | |
|---|---|---|---|---|---|
| Part A | Diethylhexyl Adipate | 15.00 | 20.00 | 10.00 | 15.00 |
| | Dicaprylyl Carbonate | 5.00 | 5.00 | 5.00 | 5.00 |
| | Homosalate | 10.00 | 8.00 | 10.00 | 8.00 |
| | Octocrylene | 9.00 | 5.00 | 8.00 | 6.00 |
| | Ethylhexyl Salicylate | 5.00 | 5.00 | 5.00 | 5.00 |
| | Butyl Methoxy- | 4.00 | 2.00 | 2.00 | 3.00 |
| | dibenzoylmethane | | | | |
| | Bis-Ethylhexyloxyphenol | 1.50 | 2.00 | 3.00 | 2.00 |
| | Methoxyphenyl Triazine | | | | |
| Part B | Cyclomethicone | 5.00 | 5.00 | 5.00 | 5.00 |
| | Pyranopyranone | 0.10 | 0.20 | 0.30 | 0.40 |
| | derivative (Table 1) | | | | |
| | BIOCIDE/ | 0.40 | 0.30 | 0.20 | 0.10 |
| | PRESERVATIVE | | | | |
| | (Table 2) | | | | |
| Part C | Alcohol (Ethanol 96%) | Qsp 100 | Qsp 100 | Qsp 100 | Qsp 100 |
| | Butane (N-Butane 1.2 | 30.00 | 30.00 | 30.00 | 30.00 |
| | Bar) (Propellant) | | | | |

### Manufacturing Instruction

Mix part A and heat up to 80° C. to melt TINOSORB® S.

Cool down to 40° C., then add part B, then C under stirring.

Fill cans with the formulation (70%) and charge with propellant (30%)

W/O Emulsion

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Butylen Glycol Dicaprylate/ | 5.50 | 5.50 | 5.50 | 5.50 | 5.50 | 5.50 | 5.50 | 5.50 | 5.50 |
| Dicaprate | | | | | | | | | |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Dimethyl Capramide | 0.50 | 0.50 | 0.50 | 0.50 | 0.50 | 0.50 | 0.50 | 0.50 | 0.50 |
| C18-36 Acid Triglyceride | 0.50 | 0.50 | 0.50 | 0.50 | 0.50 | 0.50 | 0.50 | 0.50 | 0.50 |
| Isopropyl Stearate | 3.50 | 3.50 | 3.50 | 3.50 | 3.50 | 3.50 | 3.50 | 3.50 | 3.50 |
| C12-15 Alkyl Benzoate | 4.00 | 4.00 | 4.00 | 4.00 | 4.00 | 4.00 | 4.00 | 4.00 | 4.00 |
| Polyglyceryl-2 Dipolyhydroxystearate | 5.00 | 5.00 | 5.00 | 5.00 | 5.00 | 5.00 | 5.00 | 5.00 | 5.00 |
| Diethylhexyl Butamido Triazone | 0.50 | 0.50 | 0.50 | 0.50 | 0.50 | 0.50 | 0.50 | 0.50 | 0.50 |
| Cetyl Dimethicone | 0.50 | 0.50 | 0.50 | 0.50 | 0.50 | 0.50 | 0.50 | 0.50 | 0.50 |
| Bis-Ethylhexyloxyphenol Methoxy- | 0.00 | 2.50 | 5.00 | 10.00 | 0.00 | 2.50 | 5.00 | 10.00 | 0.00 |
| phenyl Triazine (Tinosorb S) | | | | | | | | | |
| Ethylhexyl Methoxycinnamate | 0.00 | 5.00 | 10.00 | 0.00 | 5.00 | 10.00 | 0.00 | 5.00 | 10.00 |
| Tris-Biphenyl Triazine | 10.00 | 5.00 | 0.00 | 10.00 | 5.00 | 0.00 | 10.00 | 5.00 | 0.00 |
| Water | Qs to | Qs to | Qs to | Qs to | Qs to | Qs to | Qs to | Qs to | Qs to |
| | 100 | 100 | 100 | 100 | 100 | 100 | 100 | 100 | 100 |
| Butylene Glycol | 3.50 | 3.50 | 3.50 | 3.50 | 3.50 | 3.50 | 3.50 | 3.50 | 3.50 |
| Glycerin | 3.50 | 3.50 | 3.50 | 3.50 | 3.50 | 3.50 | 3.50 | 3.50 | 3.50 |
| Aluminum Starch Octenylsuccinate | 0.40 | 0.40 | 0.40 | 0.40 | 0.40 | 0.40 | 0.40 | 0.40 | 0.40 |
| Magnesium Sulfate Heptahydrate | 0.30 | 0.30 | 0.30 | 0.30 | 0.30 | 0.30 | 0.30 | 0.30 | 0.30 |
| Alcohol Denatured | 3.00 | 3.00 | 3.00 | 3.00 | 3.00 | 3.00 | 3.00 | 3.00 | 3.00 |
| BHT | 0.05 | 0.05 | 0.05 | 0.05 | 0.05 | 0.05 | 0.05 | 0.05 | 0.05 |
| Tocopheryl Acetate | 1.00 | 1.00 | 1.00 | 1.00 | 1.00 | 1.00 | 1.00 | 1.00 | 1.00 |
| Pyranopyranone derivative (Table | 0.10 | 0.25 | 0.50 | 0.10 | 0.25 | 0.50 | 0.10 | 0.25 | 0.50 |
| 1) | | | | | | | | | |
| BIOCIDE/PRESERVATIVE (Table | 0.00 | 0.00 | 0.00 | 0.25 | 0.25 | 0.25 | 0.50 | 0.50 | 0.50 |
| 2) | | | | | | | | | |

Exfoliating w/o lotion with AHA

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Water | qs. 100 | qs. 100 | qs. 100 | qs. 100 | qs. 100 | qs. 100 | qs. 100 | qs. 100 | qs. 100 |
| Quaternium-82 | 2.00 | 2.00 | 2.00 | 2.00 | 2.00 | 2.00 | 2.00 | 2.00 | 2.00 |
| PEG-12 laurate | 0.50 | 0.50 | 0.50 | 0.50 | 0.50 | 0.50 | 0.50 | 0.50 | 0.50 |
| Cyclomethicone | 20.00 | 20.00 | 20.00 | 20.00 | 20.00 | 20.00 | 20.00 | 20.00 | 20.00 |
| Lauryl alcohol | 1.50 | 1.50 | 1.50 | 1.50 | 1.50 | 1.50 | 1.50 | 1.50 | 1.50 |
| Cetyl dimethicone copolyol | 1.00 | 1.00 | 1.00 | 1.00 | 1.00 | 1.00 | 1.00 | 1.00 | 1.00 |
| Glycolic acid | 7.20 | 7.20 | 7.20 | 7.20 | 7.20 | 7.20 | 7.20 | 7.20 | 7.20 |
| Color | qs | qs | qs | qs | qs | qs | qs | qs | qs |
| Fragance | qs | qs | qs | qs | qs | qs | qs | qs | qs |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

W/O Lotion

| | % w/w | | | |
|---|---|---|---|---|
| INCI-Name | (as supplied) | | | |
| PEG-7 Hydrogenated | 3.00 | 3.00 | 3.00 | 3.00 |
| Castor Oil | | | | |
| Polyglyceryl-3 | 4.00 | 4.00 | 4.00 | 4.00 |
| Diisostearate | | | | |
| Microcrystalline Wax | 1.00 | 1.00 | 1.00 | 1.00 |
| Magnesium Stearate | 1.50 | 1.50 | 1.50 | 1.50 |
| Mineral Oil | 15.00 | 15.00 | 15.00 | 5.00 |
| Octyldodecanol | 8.00 | 8.00 | 8.00 | 8.00 |
| Water | qs to 100 | qs to 100 | qs to 100 | qs to 100 |
| Water (and) Citric Acid | 0.05 | 0.05 | 0.05 | 0.05 |
| Magnesium Sulfate | 0.50 | 0.50 | 0.50 | 0.50 |
| Pyranopyranone | 0.10 | 0.20 | 0.30 | 0.40 |
| derivative (Table 1) | | | | |
| BIOCIDE/ | 0.40 | 0.30 | 0.20 | 0.10 |
| PRESERVATIVE | | | | |
| (Table 2) | | | | |
| Sclerotium Gum | 0.50 | 2.50 | 5.00 | 10.00 |
| Fragrance | qs | qs | qs | qs |

Water resistant W/O emulsion

| | % w/w | | | |
|---|---|---|---|---|
| INCI-Name | (as supplied) | | | |
| Polyglyceryl-10 | 2.50 | 3.00 | 3.00 | 3.00 |
| Pentastearate (and) | | | | |
| Behenyl Alcohol (and) | | | | |
| Sodium Stearoyl | | | | |
| Lactylate | | | | |
| VP/Eicosene Copolymer | 1.50 | 1.50 | 1.50 | 1.50 |
| Stearyl Alcohol | 1.50 | 1.50 | 1.50 | 1.50 |
| Squalane | 4.00 | 4.00 | 4.00 | 4.00 |
| C12-15 Alkyl Benzoate | 5.50 | 5.50 | 5.50 | 5.50 |
| Water | qs to 100 | qs to 100 | qs to 100 | qs to 100 |
| Glycerin | 1.80 | 1.80 | 1.80 | 1.80 |
| Steareth-10 Allyl Ether/ | 0.80 | 0.80 | 0.80 | 0.80 |
| Acrylates Copolymer | | | | |
| Pyranopyranone | 0.10 | 0.20 | 0.30 | 0.40 |
| derivative (Table 1) | | | | |
| BIOCIDE/ | 0.40 | 0.30 | 0.20 | 0.10 |
| PRESERVATIVE | | | | |
| (Table 2) | | | | |
| VP/Hexadecene | 2.70 | 2.70 | 2.70 | 2.70 |
| Copolymer | | | | |
| Cyclomethicone | 1.50 | 1.50 | 1.50 | 1.50 |
| Aqua (and) Tocopheryl | 3.50 | 3.50 | 3.50 | 3.50 |
| Acetate (and) Caprylic/ | | | | |
| Capric Triglyceride (and) | | | | |
| Polysorbate 80 (and) | | | | |
| Lecithin | | | | |
| Fragrance | qs | qs | qs | qs |
| Sodium Hydroxide | qs | qs | qs | qs |

Skin Protection Sunscreen W/O

| | % w/w | | | |
|---|---|---|---|---|
| INCI-Name | (as supplied) | | | |
| Glyceryl Oleate | 4.50 | 4.50 | 4.50 | 4.50 |
| PEG-7 Hydrogenated | 1.50 | 1.50 | 1.50 | 1.50 |
| Castor Oil | | | | |
| Hydrogenated Castor Oil | 0.50 | 0.50 | 0.50 | 0.50 |
| Microcrystalline Wax | 2.00 | 2.00 | 2.00 | 2.00 |
| Titanium Dioxide (and) | 8.10 | 8.10 | 8.10 | 8.10 |
| Stearic Acid (and) | | | | |
| Alumina | | | | |
| Beeswax | 1.50 | 1.50 | 1.50 | 1.50 |
| C12-15 Alkyl Benzoate | 3.00 | 3.00 | 3.00 | 3.00 |
| Isopropyl Isostearate | 2.50 | 2.50 | 2.50 | 2.50 |
| Mineral Oil | 5.00 | 5.00 | 5.00 | 5.00 |
| Aqua | qs to 100 | qs to 100 | qs to 100 | qs to 100 |
| Magnesium Sulfate | 0.50 | 0.50 | 0.50 | 0.50 |
| Citric Acid | 0.05 | 0.05 | 0.05 | 0.05 |
| Pyranopyranone | 0.10 | 0.20 | 0.30 | 0.40 |
| derivative (Table 1) | | | | |
| BIOCIDE/ | 0.40 | 0.30 | 0.20 | 0.10 |
| PRESERVATIVE | | | | |
| (Table 2) | | | | |

W/O Sunscreen Emulsion

| | % w/w | | | |
|---|---|---|---|---|
| INCI-Name | (as supplied) | | | |
| Isododecane | 8.00 | 8.00 | 8.00 | 8.00 |
| C12-15 Alkyl Benzoate | 4.00 | 4.00 | 4.00 | 4.00 |
| PEG-30 Dipolyhydroxy- | 3.00 | 3.00 | 3.00 | 3.00 |
| stearate | | | | |
| PEG-45/Dodecyl | 2.00 | 2.00 | 2.00 | 2.00 |
| Glycol Copolymer | | | | |
| Hydrogenated Castor Oil | 1.00 | 1.00 | 1.00 | 1.00 |
| Tribehenin | 1.00 | 1.00 | 1.00 | 1.00 |
| Titanium Dioxide/ | 12.50 | 12.50 | 12.50 | 12.50 |
| C12-15 Alkyl | | | | |
| Benzoate/ | | | | |
| Polyhydroystearic | | | | |
| Acid/Alumina/Silica | | | | |
| Aqua | Qs to 100 | Qs to 100 | Qs to 100 | Qs to 100 |
| Glycerin | 7.00 | 7.00 | 7.00 | 7.00 |
| Pyranopyranone | 0.10 | 0.20 | 0.30 | 0.40 |
| derivative (Table 1) | | | | |
| BIOCIDE/ | 0.40 | 0.30 | 0.20 | 0.10 |
| PRESERVATIVE | | | | |
| (Table 2) | | | | |
| Sclerotium Gum | 0.50 | 2.50 | 5.00 | 10.00 |
| Quaternium-18 Hectorite | 1.50 | 1.50 | 1.50 | 1.50 |
| Silica | 0.80 | 0.80 | 0.80 | 0.80 |
| Cyclomethicone | 6.50 | 6.50 | 6.50 | 6.50 |
| Dimethicone | 2.50 | 2.50 | 2.50 | 2.50 |

Light W/O Lotion

| Part | Ingredient | Ex. 1 | Ex. 2 | Ex. 3 | Ex. 4 |
|---|---|---|---|---|---|
| A | ABIL ® EM 90 (Cetyl | 2.0 | 2.0 | 2.0 | 2.0 |
| | PEG/PPG-10/1 | | | | |
| | Dimethicone) | | | | |
| A | TEGOSOFT ® DEC | 11.5 | 11.5 | 11.5 | 11.5 |
| | (Diethylhexyl Carbonate) | | | | |
| A | Beeswax | 0.5 | 0.5 | 0.5 | 0.5 |
| A | Hydrogenated Castor Oil | 0.5 | 0.5 | 0.5 | 0.5 |
| A | Tocopheryl Acetate | 0.5 | 0.5 | 0.5 | 0.5 |
| B | GluCare ® S (Sodium | 0.2 | 0.2 | 0.2 | 0.2 |
| | Carboxymethyl Betaglucan) | | | | |
| B | Allantoin | 0.1 | 0.1 | 0.1 | 0.1 |
| B | Glycerin | 1.0 | 1.0 | 1.0 | 1.0 |
| B | Sodium Chloride | 0.5 | 0.5 | 0.5 | 0.5 |
| B | Water | Qsp 100 | Qsp 100 | Qsp 100 | Qsp 100 |
| B | Pyranopyranone | 0.10 | 0.20 | 0.30 | 0.40 |
| | derivative (Table 1) | | | | |
| B | BIOCIDE/ | 0.40 | 0.30 | 0.20 | 0.10 |
| | PRESERVATIVE | | | | |
| | (Table 2) | | | | |
| B | Sclerotium Gum | 0.50 | 2.50 | 5.00 | 10.00 |
| C | Fragrance | q.s. | q.s. | q.s. | q.s. |

W/ Si emulsion

| | | Ex. 1 | Ex. 2 | Ex. 3 | Ex. 4 |
|---|---|---|---|---|---|
| Part | Ingredient | Wt. % | | | |
| A | Masil 756 CG (BASF) | 0.90 | 0.90 | 0.90 | 0.90 |
| | (tetrabutoxypropyl | | | | |
| | methicone) | | | | |
| A | Luvitol BL (BASF) | 2.00 | 2.00 | 2.00 | 2.00 |
| | (benzyl laurate) | | | | |
| A | Cetyl dimethicone | 2.00 | 2.00 | 2.00 | 2.00 |
| A | Polyglyceryl-4, isostearate, | 6.00 | 6.00 | 6.00 | 6.00 |
| | cetyl dimethicone | | | | |
| | copolyol and hexyl laurate | | | | |
| A | Cremophor GO 32 (BASF) | 0.60 | 0.60 | 0.60 | 0.60 |
| | (polyglyceryl-3 dioleate) | | | | |
| A | Glyceryl tribehenate | 3.00 | 3.00 | 3.00 | 3.00 |
| B | Masil SF V5 CG (BASF) | 8.85 | 8.85 | 8.85 | 8.85 |
| | (cyclomethicone) | | | | |
| B | Microfine zinc oxide | 2.00 | 2.00 | 2.00 | 2.00 |
| C | Deionized water | Qsp 100 | Qsp 100 | Qsp 100 | Qsp 100 |
| C | Xanthan gum NF | 0.10 | 0.10 | 0.10 | 0.10 |
| C | Disodium EDTA | 0.10 | 0.10 | 0.10 | 0.10 |
| C | Sodium chloride | 1.00 | 1.00 | 1.00 | 1.00 |
| C | Glycerin | 3.00 | 3.00 | 3.00 | 3.00 |
| C | Pyranopyranone | 0.10 | 0.20 | 0.30 | 0.40 |
| | derivative (Table 1) | | | | |
| | BIOCIDE/ | 0.40 | 0.30 | 0.20 | 0.10 |
| | PRESERVATIVE | | | | |
| | (Table 2) | | | | |
| C | Sclerotium Gum | 0.50 | 2.50 | 5.00 | 10.00 |
| D | Panthenol USP (BASF) | 0.10 | 0.10 | 0.10 | 0.10 |
| | (panthenol) | | | | |
| D | Sodium ascorbyl phosphate | 0.20 | 0.20 | 0.20 | 0.20 |
| E | Vitamin E Acetate C | 0.50 | 0.50 | 0.50 | 0.50 |
| | (BASF) (tocopheryl | | | | |
| | acetate) | | | | |
| E | Phytantriol | 0.30 | 0.30 | 0.30 | 0.30 |
| E | Fragrance | q.s. | q.s. | q.s. | q.s. |

Water/Silicone Sunscreen Lotion

| | | Ex. 1 | Ex. 2 | Ex. 3 | Ex. 4 |
|---|---|---|---|---|---|
| Part | Ingredient | Wt. % | | | |
| A | Polyglyceryl-4 isostearate (and) cety | 10.00 | 10.00 | 10.00 | 10.00 |
| | dimethicone copolyol (and) hexyl | | | | |
| | laurate | | | | |
| A | Cremophor GS 32 (BASF) | 0.60 | 0.60 | 0.60 | 0.60 |
| | (polyglyceryl-3 distearate) | | | | |
| A | Glyceryl tribehenate | 3.00 | 3.00 | 3.00 | 3.00 |
| A | Cetyl methicone | 2.00 | 2.00 | 2.00 | 2.00 |
| A | C12-15 alkyl benzoate | 5.00 | 5.00 | 5.00 | 5.00 |
| B | Cyclomethicone | 8.85 | 8.85 | 8.85 | 8.85 |
| B | Z-Cote HP-1 (BASF) (zinc oxide | 5.00 | 5.00 | 5.00 | 5.00 |
| | (and) dimethicone) | | | | |
| B | Phenyl trimethicone | 7.90 | 7.90 | 7.90 | 7.90 |
| B | Uvinul TiO2 (BASF) (titanium | 3.00 | 3.00 | 3.00 | 3.00 |
| | dioxide (and) trimethoxycapryly- | | | | |
| | silane) | | | | |
| C | Xanthan gum NF | 0.10 | 0.10 | 0.10 | 0.10 |
| C | Glycerin | 3.00 | 3.00 | 3.00 | 3.00 |
| D | Deionized water | Qsp | Qsp | Qsp | Qsp |
| | | 100 | 100 | 100 | 100 |
| D | Disodium EDTA | 0.10 | 0.10 | 0.10 | 0.10 |
| D | Pyranopyranone derivative (Table 1) | 0.10 | 0.20 | 0.30 | 0.40 |
| | BIOCIDE/PRESERVATIVE | 0.40 | 0.30 | 0.20 | 0.10 |
| | (Table 2) | | | | |
| D | Sclerotium Gum | 0.50 | 2.50 | 5.00 | 10.00 |
| D | Sodium chloride | 1.00 | 1.00 | 1.00 | 1.00 |
| E | Vitamin E acetate C (BASF) | 0.50 | 0.50 | 0.50 | 0.50 |
| | (tocopherol acetate) | | | | |
| F | Fragrance | q.s. | q.s. | q.s. | q.s. |

W/O/W Multiple Emulsion

| | | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |

I. Primary emulsion W/O:

| | | | | | | | | | |
|---|---|---|---|---|---|---|---|---|---|
| Isohexadecane | 15.00 | 15.00 | 15.00 | 15.00 | 15.00 | 15.00 | 15.00 | 15.00 | 15.00 |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Triisodecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| Caprylic/capric triglycerides | 4.00 | 4.00 | 4.00 | 4.00 | 4.00 | 4.00 | 4.00 | 4.00 | 4.00 |
| PPG-15 stearyl ether | 7.50 | 7.50 | 7.50 | 7.50 | 7.50 | 7.50 | 7.50 | 7.50 | 7.50 |
| PEG-30 | 4.00 | 4.00 | 4.00 | 4.00 | 4.00 | 4.00 | 4.00 | 4.00 | 4.00 |
| Dipolyhydroxystearate | | | | | | | | | |
| Bis- | 0.00 | 2.50 | 5.00 | 10.00 | 0.00 | 2.50 | 5.00 | 10.00 | 0.00 |
| Ethylhexyloxyphenol | | | | | | | | | |
| Methoxyphenyl Triazine | | | | | | | | | |
| (Tinosorb S) | | | | | | | | | |
| Octocrylene | 0.00 | 5.00 | 10.00 | 0.00 | 5.00 | 10.00 | 0.00 | 5.00 | 10.00 |
| Methylene Bis-Benzo- | 10.00 | 5.00 | 0.00 | 10.00 | 5.00 | 0.00 | 10.00 | 5.00 | 0.00 |
| triazolyl | | | | | | | | | |
| Tetramethylbutylphenol | | | | | | | | | |
| (and) water | | | | | | | | | |
| (and) decyl glucoside | | | | | | | | | |
| (and) propylene glycol | | | | | | | | | |
| (and) xanthan gum | | | | | | | | | |
| (Tinosorb M) | | | | | | | | | |
| Water | Qs to 100 | Qs to 100 | Qs to 100 | Qs to 100 | Qs to 100 | Qs to 100 | Qs to 100 | Qs to 100 | Qs to 100 |
| Sodium chloride | 0.40 | 0.40 | 0.40 | 0.40 | 0.40 | 0.40 | 0.40 | 0.40 | 0.40 |
| Preservative | q.s. | q.s. | q.s. | q.s. | q.s. | q.s. | q.s. | q.s. | q.s. |

II. Secondary emulsion W/O/W:

| | | | | | | | | | |
|---|---|---|---|---|---|---|---|---|---|
| Primary emulsion (W/O) | 70.00 | 70.00 | 70.00 | 70.00 | 70.00 | 70.00 | 70.00 | 70.00 | 70.00 |
| Water | Qs to 100 | Qs to 100 | Qs to 100 | Qs to 100 | Qs to 100 | Qs to 100 | Qs to 100 | Qs to 100 | Qs to 100 |
| Poloxamer 407 | 2.00 | 2.00 | 2.00 | 2.00 | 2.00 | 2.00 | 2.00 | 2.00 | 2.00 |
| (2-{4-[2-(4-Diethylamino-2- | 2.00 | 2.00 | 2.00 | 2.00 | 2.00 | 2.00 | 2.00 | 2.00 | 2.00 |
| hydroxy-benzoyl)-benzoyl]- | | | | | | | | | |
| piperazine-1-carbonyl}- | | | | | | | | | |
| phenyl)-(4-diethylamino-2- | | | | | | | | | |
| hydroxy-phenyl)- | | | | | | | | | |
| nnethanone | | | | | | | | | |
| Carbomer | 0.50 | 0.50 | 0.50 | 0.50 | 0.50 | 0.50 | 0.50 | 0.50 | 0.50 |
| Sodium chloride | 0.20 | 0.20 | 0.20 | 0.20 | 0.20 | 0.20 | 0.20 | 0.20 | 0.20 |
| Triethanolamine | 0.50 | 0.50 | 0.50 | 0.50 | 0.50 | 0.50 | 0.50 | 0.50 | 0.50 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

W/O/W Multiple Emulsion

| | | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |

I. Primary emulsion W/O:

| | | | | | | | | | |
|---|---|---|---|---|---|---|---|---|---|
| Isohexadecane | 15.00 | 15.00 | 15.00 | 15.00 | 15.00 | 15.00 | 15.00 | 15.00 | 15.00 |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Triisodecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| Caprylic/capric triglycerides | 4.00 | 4.00 | 4.00 | 4.00 | 4.00 | 4.00 | 4.00 | 4.00 | 4.00 |
| PPG-15 stearyl ether | 7.50 | 7.50 | 7.50 | 7.50 | 7.50 | 7.50 | 7.50 | 7.50 | 7.50 |
| PEG-30 | 4.00 | 4.00 | 4.00 | 4.00 | 4.00 | 4.00 | 4.00 | 4.00 | 4.00 |
| Dipolyhydroxystearate | | | | | | | | | |
| Ethylhexyl Triazone | 2.50 | 2.50 | 2.50 | 2.50 | 2.50 | 5.00 | 5.00 | 5.00 | 5.00 |
| Methylene Bis-Benzo- | 4.00 | 6.00 | 8.00 | 10.00 | 12.00 | 4.00 | 6.00 | 8.00 | 10.00 |
| triazolyl | | | | | | | | | |
| Tetramethylbutylphenol | | | | | | | | | |
| (and) water | | | | | | | | | |
| (and) decyl glucoside | | | | | | | | | |
| (and) propylene glycol | | | | | | | | | |
| (and) xanthan gum | | | | | | | | | |
| (Tinosorb M) | | | | | | | | | |
| Water | Qs to 100 | Qs to 100 | Qs to 100 | Qs to 100 | Qs to 100 | Qs to 100 | Qs to 100 | Qs to 100 | Qs to 100 |
| Sodium chloride | 0.40 | 0.40 | 0.40 | 0.40 | 0.40 | 0.40 | 0.40 | 0.40 | 0.40 |

II. Secondary emulsion W/O/W:

| | | | | | | | | | |
|---|---|---|---|---|---|---|---|---|---|
| Primary emulsion (W/O) | 70.00 | 70.00 | 70.00 | 70.00 | 70.00 | 70.00 | 70.00 | 70.00 | 70.00 |
| Water | Qs to 100 | Qs to 100 | Qs to 100 | Qs to 100 | Qs to 100 | Qs to 100 | Qs to 100 | Qs to 100 | Qs to 100 |
| Poloxamer 407 | 2.00 | 2.00 | 2.00 | 2.00 | 2.00 | 2.00 | 2.00 | 2.00 | 2.00 |
| Titanium Dioxide | 2.50 | 2.50 | 2.50 | 2.50 | 2.50 | 2.50 | 2.50 | 2.50 | 2.50 |
| Carbomer | 0.50 | 0.50 | 0.50 | 0.50 | 0.50 | 0.50 | 0.50 | 0.50 | 0.50 |
| Sodium chloride | 0.20 | 0.20 | 0.20 | 0.20 | 0.20 | 0.20 | 0.20 | 0.20 | 0.20 |
| Triethanolamine | 0.50 | 0.50 | 0.50 | 0.50 | 0.50 | 0.50 | 0.50 | 0.50 | 0.50 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Lipstick

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Caprylic/Capric Triglyceride | 12.00 | 12.00 | 12.00 | 12.00 | 12.00 | 12.00 | 12.00 | 12.00 | 12.00 |
| Octyldodecanol | 7.00 | 7.00 | 7.00 | 7.00 | 7.00 | 7.00 | 7.00 | 7.00 | 7.00 |
| Phenethyl Benzoate | 4.00 | 4.00 | 4.00 | 4.00 | 4.00 | 4.00 | 4.00 | 4.00 | 4.00 |
| Triisodecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| Pentaerythrityl | 10.00 | 10.00 | 10.00 | 10.00 | 10.00 | 10.00 | 10.00 | 10.00 | 10.00 |
| Tetraisostearate | | | | | | | | | |
| Polyglyceryl-3 | 2.50 | 2.50 | 2.50 | 2.50 | 2.50 | 2.50 | 2.50 | 2.50 | 2.50 |
| Diisostearate | | | | | | | | | |
| Bis-Diglyceryl | 9.00 | 9.00 | 9.00 | 9.00 | 9.00 | 9.00 | 9.00 | 9.00 | 9.00 |
| Polyacyladipate-2 | | | | | | | | | |
| Cetearyl Alcohol | 8.00 | 8.00 | 8.00 | 8.00 | 8.00 | 8.00 | 8.00 | 8.00 | 8.00 |
| Myristyl Myristate | 3.50 | 3.50 | 3.50 | 3.50 | 3.50 | 3.50 | 3.50 | 3.50 | 3.50 |
| Beeswax | 5.00 | 5.00 | 5.00 | 5.00 | 5.00 | 5.00 | 5.00 | 5.00 | 5.00 |
| Copernicia Cerifera | 1.50 | 1.50 | 1.50 | 1.50 | 1.50 | 1.50 | 1.50 | 1.50 | 1.50 |
| (Carnauba) Wax | | | | | | | | | |
| Cera Alba | 0.50 | 0.50 | 0.50 | 0.50 | 0.50 | 0.50 | 0.50 | 0.50 | 0.50 |
| Tocopheryl Acetate | 0.50 | 0.50 | 0.50 | 0.50 | 0.50 | 0.50 | 0.50 | 0.50 | 0.50 |
| Tocopherol; Ascorbyl | 0.05 | 0.05 | 0.05 | 0.05 | 0.05 | 0.05 | 0.05 | 0.05 | 0.05 |
| Palmitate | | | | | | | | | |
| Simmondsia Chinensis | 2.00 | 2.00 | 2.00 | 2.00 | 2.00 | 2.00 | 2.00 | 2.00 | 2.00 |
| (Jojoba) Seed Extract | | | | | | | | | |
| Parfume/BHT | q.s | q.s | q.s | q.s | q.s | q.s | q.s | q.s | q.s |
| Ricinus Communis | QS to 100 | QS to 100 | QS to 100 | QS to 100 | QS to 100 | QS to 100 | QS to 100 | QS to 100 | QS to 100 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Octyldodecanol | 3.00 | 3.00 | 3.00 | 3.00 | 3.00 | 3.00 | 3.00 | 3.00 | 3.00 |
| Phenethyl Benzoate | 5.00 | 5.00 | 5.00 | 5.00 | 5.00 | 5.00 | 5.00 | 5.00 | 5.00 |
| Triisodecyl Trimellitate | 1.00 | 1.00 | 1.00 | 1.00 | 1.00 | 1.00 | 1.00 | 1.00 | 1.00 |
| Dimethyl Capramide | 0.50 | 0.50 | 0.50 | 0.50 | 0.50 | 0.50 | 0.50 | 0.50 | 0.50 |
| Butylene Glycol Dicaprylate/ | 12.00 | 12.00 | 12.00 | 12.00 | 12.00 | 12.00 | 12.00 | 12.00 | 12.00 |
| Dicaprate | | | | | | | | | |
| Pentaerythrityl | 7.00 | 7.00 | 7.00 | 7.00 | 7.00 | 7.00 | 7.00 | 7.00 | 7.00 |
| Tetraisostearate | | | | | | | | | |
| Bis-Diglyceryl | 8.00 | 8.00 | 8.00 | 8.00 | 8.00 | 8.00 | 8.00 | 8.00 | 8.00 |
| Polyacyladipate-2 | | | | | | | | | |
| Cetearyl Alcohol | 7.00 | 7.00 | 7.00 | 7.00 | 7.00 | 7.00 | 7.00 | 7.00 | 7.00 |
| Myristyl Myristate | 3.00 | 3.00 | 3.00 | 3.00 | 3.00 | 3.00 | 3.00 | 3.00 | 3.00 |
| Beeswax | 6.00 | 6.00 | 6.00 | 6.00 | 6.00 | 6.00 | 6.00 | 6.00 | 6.00 |
| Copernicia Cerifera | 1.50 | 1.50 | 1.50 | 1.50 | 1.50 | 1.50 | 1.50 | 1.50 | 1.50 |
| (Carnauba) Wax | | | | | | | | | |
| Cera Alba | 0.50 | 0.50 | 0.50 | 0.50 | 0.50 | 0.50 | 0.50 | 0.50 | 0.50 |
| C20-40 Alkyl Stearate | 1.50 | 1.50 | 1.50 | 1.50 | 1.50 | 1.50 | 1.50 | 1.50 | 1.50 |
| Tocopheryl Acetate | 1.00 | 1.00 | 1.00 | 1.00 | 1.00 | 1.00 | 1.00 | 1.00 | 1.00 |
| Tocopherol; Ascorbyl | 0.05 | 0.05 | 0.05 | 0.05 | 0.05 | 0.05 | 0.05 | 0.05 | 0.05 |
| Palmitate | | | | | | | | | |
| Simmondsia Chinensis | 1.00 | 1.00 | 1.00 | 1.00 | 1.00 | 1.00 | 1.00 | 1.00 | 1.00 |
| (Jojoba) Seed Extract | | | | | | | | | |
| Parfum. BHT | q.s | q.s | q.s | q.s | q.s | q.s | q.s | q.s | q.s |
| Ricinus Communis | QS to 100 | QS to 100 | QS to 100 | QS to 100 | QS to 100 | QS to 100 | QS to 100 | QS to 100 | QS to 100 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Lip balm SPF 15

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Petrolatum | 15.00 | 15.00 | 15.00 | 15.00 | 15.00 | 15.00 | 15.00 | 15.00 | 15.00 |
| Beeswax | 8.00 | 8.00 | 8.00 | 8.00 | 8.00 | 8.00 | 8.00 | 8.00 | 8.00 |
| Candelilla wax | 12.00 | 12.00 | 12.00 | 12.00 | 12.00 | 12.00 | 12.00 | 12.00 | 12.00 |
| Cocoa seed butter | 2.00 | 2.00 | 2.00 | 2.00 | 2.00 | 2.00 | 2.00 | 2.00 | 2.00 |
| Benzophenone-3 | 3.00 | 3.00 | 3.00 | 3.00 | 3.00 | 3.00 | 3.00 | 3.00 | 3.00 |
| pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Carrot extract | 5.00 | 5.00 | 5.00 | 5.00 | 5.00 | 5.00 | 5.00 | 5.00 | 5.00 |
| Aloe barbadensis oil | 7.00 | 7.00 | 7.00 | 7.00 | 7.00 | 7.00 | 7.00 | 7.00 | 7.00 |
| Chamomile oil | 6.00 | 6.00 | 6.00 | 6.00 | 6.00 | 6.00 | 6.00 | 6.00 | 6.00 |
| Caprylic/capric triglyceride | Qs. | Qs. | Qs. | Qs. | Qs. | Qs. | Qs. | Qs. | Qs. |
| | To 100 | To 100 | To 100 | To 100 | To 100 | To 100 | To 100 | To 100 | To 100 |
| Jojoba oil | 14.20 | 14.20 | 14.20 | 14.20 | 14.20 | 14.20 | 14.20 | 14.20 | 14.20 |
| Ethylhexyl Methoxycinnamate | 7.50 | 7.50 | 7.50 | 7.50 | 7.50 | 7.50 | 7.50 | 7.50 | 7.50 |
| Peppermint oil | 0.20 | 0.20 | 0.20 | 0.20 | 0.20 | 0.20 | 0.20 | 0.20 | 0.20 |

CREAM FOUNDATION with UV Protection
Make up applications

| | | a | b | c | d |
|---|---|---|---|---|---|
| Part | Ingredient | Wt. % | | | |
| B | Deionized Water | 15.00 | 15.00 | 15.00 | 15.00 |
| B | Titanium Dioxide | 7.00 | 7.00 | 7.00 | 7.00 |
| B | Iron Oxides | 1.30 | 1.30 | 1.30 | 1.30 |
| B | Iron Oxides | 1.10 | 1.10 | 1.10 | 1.10 |
| B | Talc | 1.00 | 1.00 | 1.00 | 1.00 |
| B | Iron Oxides | 0.20 | 0.20 | 0.20 | 0.20 |
| C | Mineral Oil/Drakeol 7/Penreco | 8.00 | 8.00 | 8.00 | 8.00 |
| C | Glyceryl Stearate SE | 4.00 | 4.00 | 4.00 | 4.00 |
| C | Ethylhexyl Palmitate | 3.00 | 3.00 | 3.00 | 3.00 |
| C | Emulsifying Wax | 2.50 | 2.50 | 2.50 | 2.50 |
| C | Petrolatum/Snow Petrolatum/Penreco | 1.00 | 1.00 | 1.00 | 1.00 |
| D | Pyranopyranone derivative (Table 1) | 0.10 | 0.20 | 0.30 | 0.40 |
| | BIOCIDE/PRESERVATIVE | 0.40 | 0.30 | 0.20 | 0.10 |
| | (Table 2) | | | | |
| D | Vitamins A, C or E | 0.80 | 0.50 | — | 1.00 |
| | (free or as derivative) | | | | |
| D | Fragrance | q.s. | q.s. | q.s. | q.s. |

Foundations: Anhydrous forms

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Isononyl Isononanoate | qsp | qsp | qsp | qsp | qsp | qsp | qsp | qsp | qsp |
| Phenethyl Benzoate | 4.00 | 4.00 | 4.00 | 4.00 | 4.00 | 4.00 | 4.00 | 4.00 | 4.00 |
| Tridecyl Trimellitate | 2.00 | 2.00 | 2.00 | 2.00 | 2.00 | 2.00 | 2.00 | 2.00 | 2.00 |
| Triisodecyl Trimellitate | 2.00 | 2.00 | 2.00 | 2.00 | 2.00 | 2.00 | 2.00 | 2.00 | 2.00 |
| Dimethyl Capramide | 1.00 | 1.00 | 1.00 | 1.00 | 1.00 | 1.00 | 1.00 | 1.00 | 1.00 |
| Sorbitan Sesquioleate | 0.50 | 0.50 | 0.50 | 0.50 | 0.50 | 0.50 | 0.50 | 0.50 | 0.50 |
| Cyclopentasiloxane | 15.00 | 15.00 | 15.00 | 15.00 | 15.00 | 15.00 | 15.00 | 15.00 | 15.00 |
| Cylopentasiloxane (and) | 15.00 | 15.00 | 15.00 | 15.00 | 15.00 | 15.00 | 15.00 | 15.00 | 15.00 |
| Quaternium-18 Hectorite | | | | | | | | | |
| Talc | 5.00 | 5.00 | 5.00 | 5.00 | 5.00 | 5.00 | 5.00 | 5.00 | 5.00 |
| Iron oxides | 2.50 | 2.50 | 2.50 | 2.50 | 2.50 | 2.50 | 2.50 | 2.50 | 2.50 |
| PVP/eicosane copolymer | 1.00 | 1.00 | 1.00 | 1.00 | 1.00 | 1.00 | 1.00 | 1.00 | 1.00 |
| Tocopherol Acetate | 0.50 | 0.50 | 0.50 | 0.50 | 0.50 | 0.50 | 0.50 | 0.50 | 0.50 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Pressed powder

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Petrolatum | 1.00 | 1.00 | 1.00 | 1.00 | 1.00 | 1.00 | 1.00 | 1.00 | 1.00 |
| Titanium dioxide | 12.00 | 12.00 | 12.00 | 12.00 | 12.00 | 12.00 | 12.00 | 12.00 | 12.00 |
| Mica (and) Methicone | 30.00 | 6.00 | 6.00 | 6.00 | 6.00 | 6.00 | 6.00 | 6.00 | 6.00 |
| Mica dimethicone coated | 4.00 | 4.00 | 4.00 | 4.00 | 4.00 | 4.00 | 4.00 | 4.00 | 4.00 |
| Talc (and) Triethoxy- | 4.00 | 4.00 | 4.00 | 4.00 | 4.00 | 4.00 | 4.00 | 4.00 | 4.00 |
| caprylylsilane | | | | | | | | | |
| Talc | Qs. 100 | Qs. 100 | Qs. 100 | Qs. 100 | Qs. 100 | Qs. 100 | Qs. 100 | Qs. 100 | Qs. 100 |
| Silica | 1.00 | 1.00 | 1.00 | 1.00 | 1.00 | 1.00 | 1.00 | 1.00 | 1.00 |
| PTFE | 2.00 | 2.00 | 2.00 | 2.00 | 2.00 | 2.00 | 2.00 | 2.00 | 2.00 |
| Iron Oxides | 3.35 | 3.35 | 3.35 | 3.35 | 3.35 | 3.35 | 3.35 | 3.35 | 3.35 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Full Length Lush Mascara

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| PVP | 1.80 | 1.80 | 1.80 | 1.80 | 1.80 | 1.80 | 1.80 | 1.80 | 1.80 |
| Hydroxyethylcellulose | 0.30 | 0.30 | 0.30 | 0.30 | 0.30 | 0.30 | 0.30 | 0.30 | 0.30 |
| 40% Black iron oxide dispersed | 20.00 | 20.00 | 20.00 | 20.00 | 20.00 | 20.00 | 20.00 | 20.00 | 20.00 |
| in propylene glycol | | | | | | | | | |
| Triethanolamine | 2.00 | 2.00 | 2.00 | 2.00 | 2.00 | 2.00 | 2.00 | 2.00 | 2.00 |
| Water | 53.10 | 53.10 | 53.10 | 53.10 | 53.10 | 53.10 | 53.10 | 53.10 | 53.10 |
| Stearic Acid | 4.50 | 4.50 | 4.50 | 4.50 | 4.50 | 4.50 | 4.50 | 4.50 | 4.50 |
| Glyceryl Monostearate | 2.00 | 2.00 | 2.00 | 2.00 | 2.00 | 2.00 | 2.00 | 2.00 | 2.00 |
| Carnauba Wax | 4.00 | 4.00 | 4.00 | 4.00 | 4.00 | 4.00 | 4.00 | 4.00 | 4.00 |
| Lanolin Alcohol | 1.00 | 1.00 | 1.00 | 1.00 | 1.00 | 1.00 | 1.00 | 1.00 | 1.00 |
| Beeswax | 7.00 | 7.00 | 7.00 | 7.00 | 7.00 | 7.00 | 7.00 | 7.00 | 7.00 |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Polyquaternium-7 | 3.50 | 3.50 | 3.50 | 3.50 | 3.50 | 3.50 | 3.50 | 3.50 | 3.50 |
| Pyranopyranone derivative | 0.10 | 0.25 | 0.50 | 0.10 | 0.25 | 0.50 | 0.10 | 0.25 | 0.50 |
| (Table 1) | | | | | | | | | |

CATIONIC LOTION MAKE-UP WITH SUNSCREEN

| | | | | | Wt. |
|---|---|---|---|---|---|
| Part | Ingredient | | | | % |
| A | Laureth-23 (Brij 35 SP) | 0.50 | 0.50 | 0.50 | 0.50 |
| A | Glyceryl Stearate | 5.00 | 5.00 | 5.00 | 5.00 |
| | (Cerasynt SD) | | | | |
| A | Dimethicone | 1.00 | 1.00 | 1.00 | 1.00 |
| | (Dow Corning 200 Fluid) | | | | |
| A | Octyldodecyl Stearoyl | 4.00 | 4.00 | 4.00 | 4.00 |
| | Stearate (Ceraphyl 847) | | | | |
| A | Cetearyl Octanoate | 2.00 | 4.00 | 6.00 | 3.00 |
| | Tridecyl Trimellitate | 4.00 | 2.00 | 1.00 | 6.00 |
| A | Cetyl Alcohol | 1.00 | 1.00 | 1.00 | 1.00 |
| B-1 | Water, Deionized | Qs | Qs | Qs | Qs |
| | | 100 | 100 | 100 | 100 |
| B-1 | Hydroxyethylcellulose | 0.50 | 0.50 | 0.50 | 0.50 |
| | (Natrosol 250HR) | | | | |
| B-2 | Diethylaminoethyl Stearate | 1.00 | 1.00 | 1.00 | 1.00 |
| | (Cerasynt 303) | | | | |
| B-2 | Lactic Acid (88%) | 0.75 | 0.75 | 0.75 | 0.75 |
| B-2 | Glycerin | 2.50 | 2.50 | 2.50 | 2.50 |
| B-3 | Pigment Blend No. B73-9-1 | 13.00 | 13.00 | 13.00 | 13.00 |
| B-4 | Quaternium-26 | 1.50 | 1.50 | 1.50 | 1.50 |
| | (Ceraphyl 65) | | | | |
| | Pyranopyranone | 0.10 | 0.20 | 0.30 | 0.40 |
| | derivative (Table 1) | | | | |
| | BIOCIDE/ | 0.40 | 0.30 | 0.20 | 0.10 |
| | PRESERVATIVE (Table 2) | | | | |

Eye Make-up Remover

| | | Wt. | Wt. | Wt. | Wt. |
|---|---|---|---|---|---|
| Part | Ingredient | % | % | % | % |
| A | REWOTERIC ® AM KSF 40 | 5.00 | 5.00 | 5.00 | 5.00 |
| | (Sodium Cocoampho-propionate) | | | | |
| A | Propylene Glycol | Qs | Qs | Qs | Qs |
| | | 100 | 100 | 100 | 100 |
| A | Glycerin | 30.00 | 30.00 | 30.00 | 30.00 |
| A | Lipoxol 400 Med | 15.00 | 8.00 | 6.00 | 10.00 |
| | (PEG-8, Sasol) | | | | |
| A | Cetearyl Octanoate | 2.00 | 4.00 | 6.00 | 3.00 |
| | Tridecyl Trimellitate | 4.00 | 2.00 | 1.00 | 6.00 |
| | Pyranopyranone derivative (Table 1) | 0.10 | 0.20 | 0.30 | 0.40 |
| | BIOCIDE/PRESERVATIVE | 0.40 | 0.30 | 0.20 | 0.10 |
| | (Table 2) | | | | |

Alcohol-free anti-acne cleansing wet wipes

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| A | BIOCIDE/PRESERVATIVE (Table | 0.20 | 0.20 | 0.20 | 0.20 | 0.20 | 0.20 | 0.20 | 0.20 | 0.20 |
| | 2) | | | | | | | | | |
| B | Disodium laureth sulfosuccinate | 3.00 | 3.00 | 3.00 | 3.00 | 3.00 | 3.00 | 3.00 | 3.00 | 3.00 |
| | Lauryl glucoside | 3.00 | 3.00 | 3.00 | 3.00 | 3.00 | 3.00 | 3.00 | 3.00 | 3.00 |
| | PEG-7 glyceryl cocoate | 1.00 | 1.00 | 1.00 | 1.00 | 1.00 | 1.00 | 1.00 | 1.00 | 1.00 |
| C | Water | Qs. | Qs. | Qs. | Qs. | Qs. | Qs. | Qs. | Qs. | Qs. |
| | | To 100 | To 100 | To 100 | To 100 | To 100 | To 100 | To 100 | To 100 | To 100 |
| D | Fragance | Qs | Qs | Qs | Qs | Qs | Qs | Qs | Qs | Qs |
| | Pyranopyranone derivative (Table | 0.10 | 0.20 | 0.30 | 0.40 | 0.50 | 0.60 | 0.70 | 0.80 | 0.90 |
| | 1) | | | | | | | | | |

Anti-acne cleanser

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| A | BIOCIDE/PRESERVATIVE (Table | 0.30 | 0.30 | 0.30 | 0.30 | 0.30 | 0.30 | 0.30 | 0.30 | 0.30 |
| | 2) | | | | | | | | | |
| | Salicylic acid | 2.00 | 2.00 | 2.00 | 2.00 | 2.00 | 2.00 | 2.00 | 2.00 | 2.00 |
| B | Sodium laureth sulfate | 28.60 | 28.60 | 28.60 | 28.60 | 28.60 | 28.60 | 28.60 | 28.60 | 28.60 |
| | Decyl glucoside | 5.00 | 5.00 | 5.00 | 5.00 | 5.00 | 5.00 | 5.00 | 5.00 | 5.00 |
| | Cocoamidopropyl betaine | 7.90 | 7.90 | 7.90 | 7.90 | 7.90 | 7.90 | 7.90 | 7.90 | 7.90 |
| | PEG-150 distearate | 1.00 | 1.00 | 1.00 | 1.00 | 1.00 | 1.00 | 1.00 | 1.00 | 1.00 |
| C | Water | Qs. 100 | Qs. 100 | Qs. 100 | Qs. 100 | Qs. 100 | Qs. 100 | Qs. 100 | Qs. 100 | Qs. 100 |
| D | Polyquaternium-7 | 1.25 | 1.25 | 1.25 | 1.25 | 1.25 | 1.25 | 1.25 | 1.25 | 1.25 |
| | Glycerin | 1.00 | 1.00 | 1.00 | 1.00 | 1.00 | 1.00 | 1.00 | 1.00 | 1.00 |
| | Panthenol | 0.30 | 0.30 | 0.30 | 0.30 | 0.30 | 0.30 | 0.30 | 0.30 | 0.30 |
| | Fragance | qs | qs | qs | qs | qs | qs | qs | qs | qs |
| | Sodium Chloride | 3.00 | 3.00 | 3.00 | 3.00 | 3.00 | 3.00 | 3.00 | 3.00 | 3.00 |
| | Pyranopyranone derivative (Table | 0.10 | 0.20 | 0.30 | 0.40 | 0.50 | 0.60 | 0.70 | 0.80 | 0.90 |
| | 1) | | | | | | | | | |
| E | Water (and) sodium hydroxide, | qs | qs | qs | qs | qs | qs | qs | qs | qs |
| | soln 10% to pH 5.50 | | | | | | | | | |

Anti-Acne Stick

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Octyldodecanol | 32.00 | 32.00 | 32.00 | 32.00 | 32.00 | 32.00 | 32.00 | 32.00 | 32.00 |
| Caprylic/Capric Triglyceride | 21.40 | 21.40 | 21.40 | 21.40 | 21.40 | 21.40 | 21.40 | 21.40 | 21.40 |
| Bis-Diglycerylcapry-late/- | 8.00 | 8.00 | 8.00 | 8.00 | 8.00 | 8.00 | 8.00 | 8.00 | 8.00 |
| aprate/Isostearate/Steaate/ | | | | | | | | | |
| Hydroxystearate Adipate | | | | | | | | | |
| Butyrospermum Parkii (Shea | 5.00 | 5.00 | 5.00 | 5.00 | 5.00 | 5.00 | 5.00 | 5.00 | 5.00 |
| Butter) | | | | | | | | | |
| Caprylic/Capric/Myristic/Stearic | 5.00 | 5.00 | 5.00 | 5.00 | 5.00 | 5.00 | 5.00 | 5.00 | 5.00 |
| Triglyceride | | | | | | | | | |
| Tetradibutyl Pentaerithrityl | 0.10 | 0.10 | 0.10 | 0.10 | 0.10 | 0.10 | 0.10 | 0.10 | 0.10 |
| Hydroxyhydrocinnamate | | | | | | | | | |
| Euphorbia Cerifera (Candelilla) | 6.50 | 6.50 | 6.50 | 6.50 | 6.50 | 6.50 | 6.50 | 6.50 | 6.50 |
| Wax | | | | | | | | | |
| Carnauba Wax | 4.50 | 4.50 | 4.50 | 4.50 | 4.50 | 4.50 | 4.50 | 4.50 | 4.50 |
| Beeswax | 12.00 | 12.00 | 12.00 | 12.00 | 12.00 | 12.00 | 12.00 | 12.00 | 12.00 |
| Hydrogenated Coco- | 5.00 | 5.00 | 5.00 | 5.00 | 5.00 | 5.00 | 5.00 | 5.00 | 5.00 |
| Glycerides | | | | | | | | | |
| Pyranopyranone derivative | 0.10 | 0.20 | 0.30 | 0.40 | 0.50 | 0.60 | 0.70 | 0.80 | 0.90 |
| (Table 1) | | | | | | | | | |
| BIOCIDE/PRESERVATIVE | 0.30 | 0.30 | 0.30 | 0.30 | 0.30 | 0.30 | 0.30 | 0.30 | 0.30 |
| (Table 2) | | | | | | | | | |

Foaming antibacterial Hand soap

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| A | Sodium lauryl sulfate | 23.70 | 23.70 | 23.70 | 23.70 | 23.70 | 23.70 | 23.70 | 23.70 | 23.70 |
| B | PEG-16 macadamia glycerides | 3.40 | 3.40 | 3.40 | 3.40 | 3.40 | 3.40 | 3.40 | 3.40 | 3.40 |
| C | Cocamidopropyl betaine | 4.70 | 4.70 | 4.70 | 4.70 | 4.70 | 4.70 | 4.70 | 4.70 | 4.70 |
| D | Triclosan | 0.30 | 1.00 | 1.00 | 0.30 | 0.30 | 1.00 | 1.00 | 1.00 | 0.30 |
| | Benzophenone-4 | 0.20 | 0.20 | 0.20 | 0.20 | 0.20 | 0.20 | 0.20 | 0.20 | 0.20 |
| E | Water | Qs. 100 | Qs. 100 | Qs. 100 | Qs. 100 | Qs. 100 | Qs. 100 | Qs. 100 | Qs. 100 | Qs. 100 |
| | Pyranopyranone derivative | 0.10 | 0.25 | 0.50 | 0.10 | 0.25 | 0.50 | 0.10 | 0.25 | 0.50 |
| | (Table 1) | | | | | | | | | |
| | BIOCIDE/PRESERVATIVE | 0.00 | 0.00 | 0.00 | 0.25 | 0.25 | 0.25 | 0.50 | 0.50 | 0.50 |
| | (Table 2) | | | | | | | | | |
| F | Hydrolyzed jojoba esters | 9.00 | 9.00 | 9.00 | 9.00 | 9.00 | 9.00 | 9.00 | 9.00 | 9.00 |
| | (and) water | | | | | | | | | |
| G | Jojoba wax PEG-120 esters | 1.50 | 1.50 | 1.50 | 1.50 | 1.50 | 1.50 | 1.50 | 1.50 | 1.50 |
| H | Color | qs | qs | qs | qs | qs | qs | qs | qs | qs |
| | Fragance | qs | qs | qs | qs | qs | qs | qs | qs | qs |

Bar Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Titanium dioxide | 0.01 | 2.00 | 5.00 | 0.50 | 2.00 | 5.00 | 1.00 | 3.00 | 4.00 |
| Stearic acid | 1.00 | 5.00 | 10.00 | 1.00 | 5.00 | 10.00 | 1.00 | 5.00 | 10.00 |
| Soap base (e.g. sodium salt | Ad | Ad | Ad | Ad | Ad | Ad | Ad | Ad | Ad |
| of tallow fatty acid or coconut | 100% | 100% | 100% | 100% | 100% | 100% | 100% | 100% | 100% |
| fally acid or glycerol) | | | | | | | | | |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Triclosan | 0 | 0.50 | 0.25 | 1.00 | 0.25 | 0 | 0 | 0.10 | 0.40 |
| Triclocarban | 2.00 | 1.00 | 0.50 | 0 | 0.80 | 0.40 | 0.20 | 0.50 | 0 |

Deodorant shower gel

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| A | BIOCIDE/PRESERVATIVE | 0.30 | 0.30 | 0.30 | 0.30 | 0.30 | 0.30 | 0.30 | 0.30 | 0.30 |
| | (Table 2) | | | | | | | | | |
| B | Sodium laureth sulfate | 28.60 | 28.60 | 28.60 | 28.60 | 28.60 | 28.60 | 28.60 | 28.60 | 28.60 |
| | Decyl glucoside | 5.00 | 5.00 | 5.00 | 5.00 | 5.00 | 5.00 | 5.00 | 5.00 | 5.00 |
| | Cocamidopropyl betaine | 7.90 | 7.90 | 7.90 | 7.90 | 7.90 | 7.90 | 7.90 | 7.90 | 7.90 |
| C | Water | qs 100 | qs 100 | qs 100 | qs 100 | qs 100 | qs 100 | qs 100 | qs 100 | qs 100 |
| D | Glycerin | 1.00 | 1.00 | 1.00 | 1.00 | 1.00 | 1.00 | 1.00 | 1.00 | 1.00 |
| | Polyquaternium-7 | 1.25 | 1.25 | 1.25 | 1.25 | 1.25 | 1.25 | 1.25 | 1.25 | 1.25 |
| | Panthenol | 0.30 | 0.30 | 0.30 | 0.30 | 0.30 | 0.30 | 0.30 | 0.30 | 0.30 |
| | Color | 0.05 | 0.05 | 0.05 | 0.05 | 0.05 | 0.05 | 0.05 | 0.05 | 0.05 |
| | Sodium benzotriazolyl butylphenol | 0.05 | 0.05 | 0.05 | 0.05 | 0.05 | 0.05 | 0.05 | 0.05 | 0.05 |
| | sulfonate (and) | | | | | | | | | |
| | buteth-3 (and) tributyl citrate | | | | | | | | | |
| | Pyranopyranone derivative | 0.10 | 0.20 | 0.30 | 0.40 | 0.50 | 0.60 | 0.70 | 0.80 | 1.00 |
| | (Table 1) | | | | | | | | | |
| | Sodium chloride | 1.00 | 1.00 | 1.00 | 1.00 | 1.00 | 1.00 | 1.00 | 1.00 | 1.00 |
| | Fragance | qs | qs | qs | qs | qs | qs | qs | qs | qs |
| E | Citric acid | qs | qs | qs | qs | qs | qs | qs | qs | qs |

Clear antibacterial hand gel

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| A | BIOCIDE/PRESERVATIVE | 0.30 | 0.20 | 0.10 | 0.00 | 0.30 | 0.30 | 0.30 | 0.30 | 0.30 |
| | (Table 2) | | | | | | | | | |
| | Glycerin | 5.00 | 5.00 | 5.00 | 5.00 | 5.00 | 5.00 | 5.00 | 5.00 | 5.00 |
| | Panthenol | 0.50 | 0.50 | 0.50 | 0.50 | 0.50 | 0.50 | 0.50 | 0.50 | 0.50 |
| | Triclosan | 0.20 | 1.00 | 1.00 | 0.50 | 0.50 | 0.50 | 1.00 | 1.00 | 0 |
| | Cyclomethicone | 0.50 | 0.50 | 0.50 | 0.50 | 0.50 | 0.50 | 0.50 | 0.50 | 0.50 |
| | SD alcohol 39-C | 50.00 | 50.00 | 50.00 | 50.00 | 50.00 | 50.00 | 50.00 | 50.00 | 50.00 |
| B | Fragance | qs | Qs | qs | qs | qs | qs | qs | qs | qs |
| C | Acrylates Copolymer | 4.50 | 4.50 | 4.50 | 4.50 | 4.50 | 4.50 | 4.50 | 4.50 | 4.50 |
| D | Water | Qsp | Qsp | Qsp | Qsp | Qsp | Qsp | Qsp | Qsp | Qsp |
| | | 100 | 100 | 100 | 100 | 100 | 100 | 100 | 100 | 100 |
| | Pyranopyranone derivative | 1.00 | 0.10 | 0.20 | 0.30 | 0.40 | 0.20 | 0.30 | 0.40 | 0.50 |
| | (Table 1) | | | | | | | | | |
| E | Water (and) sodium hydroxide, | qs | qs | qs | qs | qs | qs | qs | qs | qs |
| | soln 10% to pH 7.40 | | | | | | | | | |

Skin Liquid

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Water | qs to 100 | qs to 100 | qs to 100 | qs to 100 | qs to 100 | qs to 100 | qs to 100 | qs to 100 | qs to 100 |
| Sodium Lauryl Sulfate | 2.00 | 2.00 | 2.00 | 2.00 | 2.00 | 2.00 | 2.00 | 2.00 | 2.00 |
| PPG-2 Methyl Ether | 10.00 | 10.00 | 10.00 | 10.00 | 10.00 | 10.00 | 10.00 | 10.00 | 10.00 |
| Propylene Glycol | 10.00 | 10.00 | 10.00 | 10.00 | 10.00 | 10.00 | 10.00 | 10.00 | 10.00 |
| Color and Perfume | qs | qs | qs | qs | qs | qs | qs | qs | qs |
| Pyranopyranone derivative | 0.10 | 0.20 | 0.30 | 0.40 | 0.50 | 0.60 | 0.70 | 0.80 | 0.90 |
| (Table 1) | | | | | | | | | |
| BIOCIDE/PRESERVATIVE | 0.30 | 0.20 | 0.10 | 0.00 | 0.30 | 0.30 | 0.30 | 0.30 | 0.30 |
| (Table 2) | | | | | | | | | |
| Ethanolamine | ad pH | ad pH | ad pH | ad pH | ad pH | ad pH | ad pH | ad pH | ad pH |
| | 5.7 | 5.7 | 5.7 | 5.7 | 5.7 | 5.7 | 5.7 | 5.7 | 5.7 |

Mild Deodorant Bar Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Citric Acid | 1.50 | 1.50 | 1.50 | 1.50 | 1.50 | 1.50 | 1.50 | 1.50 | 1.50 |
| Perfume | qs | qs | qs | qs | qs | qs | qs | qs | qs |
| Pyranopyranone derivative | 0.10 | 0.20 | 0.30 | 0.40 | 0.50 | 0.60 | 0.70 | 0.80 | 0.90 |
| (Table 1) | | | | | | | | | |
| BIOCIDE/PRESERVATIVE | 0.30 | 0.20 | 0.10 | 0.00 | 0.30 | 0.30 | 0.30 | 0.30 | 0.30 |
| (Table 2) | | | | | | | | | |
| Triclosan | 1.00 | 0 | 1.00 | 1.00 | 0 | 0.50 | 0.50 | 0.50 | 1.00 |
| Disodium Lauryl | 93.20 | 93.20 | 93.20 | 93.20 | 93.20 | 93.20 | 93.20 | 93.20 | 93.20 |
| Sulfosuccinate | | | | | | | | | |
| (and) Sodium Coco- | | | | | | | | | |
| Sulfate (and) Corn Starch | | | | | | | | | |
| (and) Cetearyl Alcohol | | | | | | | | | |
| (and) Paraffin (and) Titanium | | | | | | | | | |
| Dioxide | | | | | | | | | |
| Water | qs to 100 | qs to 100 | qs to 100 | qs to 100 | qs to 100 | qs to 100 | qs to 100 | qs to 100 | qs to 100 |

Hand Scrub

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Sodium Laureth Sulfate | 17.9 | 17.9 | 17.9 | 17.9 | 17.9 | 17.9 | 17.9 | 17.9 | 17.9 |
| Cocamidopropyl Betaine | 4.00 | 4.00 | 4.00 | 4.00 | 4.00 | 4.00 | 4.00 | 4.00 | 4.00 |
| Sodium Cumene Sulfonate | 3.00 | 3.00 | 3.00 | 3.00 | 3.00 | 3.00 | 3.00 | 3.00 | 3.00 |
| Pyranopyranone derivative | 0.10 | 0.25 | 0.50 | 1.00 | 1.50 | 2.00 | 3.00 | 4.00 | 5.00 |
| (Table 1) | | | | | | | | | |
| Hydroxypropyl | 1.00 | 1.00 | 1.00 | 1.00 | 1.00 | 1.00 | 1.00 | 1.00 | 1.00 |
| Methylcellulose | | | | | | | | | |
| Water | qs to 100 | qs to 100 | qs to 100 | qs to 100 | qs to 100 | qs to 100 | qs to 100 | qs to 100 | qs to 100 |
| Sodium Benzotriazolyl | 0.05 | 0.05 | 0.05 | 0.05 | 0.05 | 0.05 | 0.05 | 0.05 | 0.05 |
| Butylphenol | | | | | | | | | |
| Sulfonate (and) | | | | | | | | | |
| Buteth-3 (and) | | | | | | | | | |
| Tributyl Citrate | | | | | | | | | |
| BIOCIDE/PRESERVATIVE | 0.30 | 0.20 | 0.10 | 0.00 | 0.30 | 0.30 | 0.30 | 0.30 | 0.30 |
| (Table 2) | | | | | | | | | |
| Triclosan | 0 | 0.50 | 0.25 | 1.00 | 0.10 | 0 | 0 | 0 | 0 |
| Triclocarban | 0 | 0 | 0 | 0 | 0 | 0.20 | 0.40 | 0.80 | 1.50 |
| D&C Red No. 33 | 0.01 | 0.01 | 0.01 | 0.01 | 0.01 | 0.01 | 0.01 | 0.01 | 0.01 |
| Ethanolamine | ad pH | ad pH | ad pH | ad pH | ad pH | ad pH | ad pH | ad pH | ad pH |
| | 5.5 | 5.5 | 5.5 | 5.5 | 5.5 | 5.5 | 5.5 | 5.5 | 5.5 |

Anti-Bacterial Liquid Hand Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| IONIC LIQ (Table 1) | 0.10 | 0.20 | 0.30 | 0.40 | 0.50 | 0.60 | 0.70 | 0.80 | 1.00 |
| Sodium Laureth Sulfate | 28.60 | 28.60 | 28.60 | 28.60 | 28.60 | 28.60 | 28.60 | 28.60 | 28.60 |
| Lauryl Glucoside | 8.00 | 8.00 | 8.00 | 8.00 | 8.00 | 8.00 | 8.00 | 8.00 | 8.00 |
| BIOCIDE/PRESERVATIVE | 0.30 | 0.20 | 0.10 | 0.00 | 0.30 | 0.30 | 0.30 | 0.30 | 0.30 |
| (Table 2) | | | | | | | | | |
| Water | qs to 100 | qs to 100 | qs to 100 | qs to 100 | qs to 100 | qs to 100 | qs to 100 | qs to 100 | qs to 100 |
| Polyquaternium-7 | 1.25 | 1.25 | 1.25 | 1.25 | 1.25 | 1.25 | 1.25 | 1.25 | 1.25 |
| FD&C Green No. 8 | 0.05 | 0.05 | 0.05 | 0.05 | 0.05 | 0.05 | 0.05 | 0.05 | 0.05 |
| Sodium Benzotriazolyl Butyl- | 0.05 | 0.05 | 0.05 | 0.05 | 0.05 | 0.05 | 0.05 | 0.05 | 0.05 |
| phenol Sulfonate (and) | | | | | | | | | |
| Buteth-3 (and) Tributyl | | | | | | | | | |
| Citrate | | | | | | | | | |
| Laureth 02 | 1.50 | 1.50 | 1.50 | 1.50 | 1.50 | 1.50 | 1.50 | 1.50 | 1.50 |
| Pyranopyranone derivative | 0.10 | 0.25 | 0.50 | 1.00 | 1.50 | 2.00 | 3.00 | 4.00 | 5.00 |
| (Table 1) | | | | | | | | | |
| Sodium Chloride | 1.00 | 1.00 | 1.00 | 1.00 | 1.00 | 1.00 | 1.00 | 1.00 | 1.00 |
| Citric Acid | ad | ad | ad | ad | ad | ad | ad | ad | ad |
| | pH | pH | pH | pH | pH | pH | pH | pH | pH |
| | 6.0 | 6.0 | 6.0 | 6.0 | 6.0 | 6.0 | 6.0 | 6.0 | 6.0 |

Moisturizing Antibacterial liquid Hand Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Water | 18.20 | 18.20 | 18.20 | 18.20 | 18.20 | 18.20 | 18.20 | 18.20 | 18.20 |
| Sodium C14-16 Olefin Sulfonate | 30.00 | 30.00 | 30.00 | 30.00 | 30.00 | 30.00 | 30.00 | 30.00 | 30.00 |
| (and) Sodium Laueth | | | | | | | | | |
| Sulfate (and) Lauramide | | | | | | | | | |
| DEA | | | | | | | | | |
| Cocamidopropyl Betaine | 10.00 | 10.00 | 10.00 | 10.00 | 10.00 | 10.00 | 10.00 | 10.00 | 10.00 |
| Glycerin | 3.00 | 3.00 | 3.00 | 3.00 | 3.00 | 3.00 | 3.00 | 3.00 | 3.00 |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Aloe Vera | 0.50 | 0.50 | 0.50 | 0.50 | 0.50 | 0.50 | 0.50 | 0.50 | 0.50 |
| Lauramide DEA | 3.00 | 3.00 | 3.00 | 3.00 | 3.00 | 3.00 | 3.00 | 3.00 | 3.00 |
| Water | 30.00 | 30.00 | 30.00 | 30.00 | 30.00 | 30.00 | 30.00 | 30.00 | 30.00 |
| Acrylamidopropyltrimonium | 0.20 | 0.20 | 0.20 | 0.20 | 0.20 | 0.20 | 0.20 | 0.20 | 0.20 |
| Chloride/Acrylamide Copoymer | | | | | | | | | |
| Polysorbate 20 | 2.00 | 2.00 | 2.00 | 2.00 | 2.00 | 2.00 | 2.00 | 2.00 | 2.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| Sodium Chloride | 1.00 | 1.00 | 1.00 | 1.00 | 1.00 | 1.00 | 1.00 | 1.00 | 1.00 |
| Citric Acid | 0.65 | 0.65 | 0.65 | 0.65 | 0.65 | 0.65 | 0.65 | 0.65 | 0.65 |

Liquid Hand Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Pyranopyranone derivative | 0.10 | 0.20 | 0.30 | 0.40 | 0.50 | 0.60 | 0.70 | 0.80 | 1.00 |
| (Table 1) | | | | | | | | | |
| Sodium Laureth Sulfate | 28.60 | 28.60 | 28.60 | 28.60 | 28.60 | 28.60 | 28.60 | 28.60 | 28.60 |
| Lauryl Glucoside | 8.00 | 8.00 | 8.00 | 8.00 | 8.00 | 8.00 | 8.00 | 8.00 | 8.00 |
| BIOCIDE/PRESERVATIVE | 0.10 | 0.25 | 0.20 | 0.30 | 0.30 | 0.30 | 0.30 | 0.30 | 0.40 |
| (Table 2) | | | | | | | | | |
| Water | qs to 100 | qs to 100 | qs to 100 | qs to 100 | qs to 100 | qs to 100 | qs to 100 | qs to 100 | qs to 100 |
| Polyquaternium-7 | 1.25 | 1.25 | 1.25 | 1.25 | 1.25 | 1.25 | 1.25 | 1.25 | 1.25 |
| FD&C Green No. 8 | 0.05 | 0.05 | 0.05 | 0.05 | 0.05 | 0.05 | 0.05 | 0.05 | 0.05 |
| Sodium Benzotriazolyl Butyl- | 0.05 | 0.05 | 0.05 | 0.05 | 0.05 | 0.05 | 0.05 | 0.05 | 0.05 |
| phenol Sulfonate (and) Bueth- | | | | | | | | | |
| 3 (and) Tributyl Citrate | | | | | | | | | |
| Laureth 02 | 1.50 | 1.50 | 1.50 | 1.50 | 1.50 | 1.50 | 1.50 | 1.50 | 1.50 |
| Sodium Chloride | 1.00 | 1.00 | 1.00 | 1.00 | 1.00 | 1.00 | 1.00 | 1.00 | 1.00 |
| Citric Acid | ad pH | ad pH | ad pH | ad pH | ad pH | ad pH | ad pH | ad pH | ad pH |
| | 6.0 | 6.0 | 6.0 | 6.0 | 6.0 | 6.0 | 6.0 | 6.0 | 6.0 |

Moisturizing liquid Hand Soap

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Sodium C14-16 Olefin Sulfonate | 30.00 | 30.00 | 30.00 | 30.00 | 30.00 | 30.00 | 30.00 | 30.00 | 30.00 |
| (and) Sodium Laureth | | | | | | | | | |
| Sulfate (and) Lauramide DEA | | | | | | | | | |
| Cocamidopropyl Betaine | 10.00 | 10.00 | 10.00 | 10.00 | 10.00 | 10.00 | 10.00 | 10.00 | 10.00 |
| Glycerin | 3.00 | 3.00 | 3.00 | 3.00 | 3.00 | 3.00 | 3.00 | 3.00 | 3.00 |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Aloe Vera | 0.50 | 0.50 | 0.50 | 0.50 | 0.50 | 0.50 | 0.50 | 0.50 | 0.50 |
| Lauramide DEA | 3.00 | 3.00 | 3.00 | 3.00 | 3.00 | 3.00 | 3.00 | 3.00 | 3.00 |
| Water | 48.20 | 48.20 | 48.20 | 48.20 | 48.20 | 48.20 | 48.20 | 48.20 | 48.20 |
| Acrylamidopropyltrimonium | 0.20 | 0.20 | 0.20 | 0.20 | 0.20 | 0.20 | 0.20 | 0.20 | 0.20 |
| Chloride/Acrylamide Copoymer | | | | | | | | | |
| Polysorbate 20 | 2.00 | 2.00 | 2.00 | 2.00 | 2.00 | 2.00 | 2.00 | 2.00 | 2.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| Sodium Chloride | 1.00 | 1.00 | 1.00 | 1.00 | 1.00 | 1.00 | 1.00 | 1.00 | 1.00 |
| Citric Acid | 0.65 | 0.65 | 0.65 | 0.65 | 0.65 | 0.65 | 0.65 | 0.65 | 0.65 |

Shampoo Shower gel

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Decyl Glucoside | 15.00 | 10.00 | 8.00 | 5.00 | 10.00 | 12.00 | 15.00 | 20.00 | 8.00 |
| Cocamidopropyl Betaine | 7.00 | 5.00 | 4.00 | 6.00 | 4.00 | 8.00 | 6.00 | 4.00 | 8.00 |
| Water | Qsp | Qsp | Qsp | Qsp | Qsp | Qsp | Qsp | Qsp | Qsp |
| | 100 | 100 | 100 | 100 | 100 | 100 | 100 | 100 | 100 |
| Piroctone Olamine | — | 0.3 | 1.00 | — | 0.5 | — | — | 0.75 | — |
| Climbazole | — | 0.5 | — | — | 0.75 | — | — | — | — |
| Zinc Pyrithion, 48% | — | 2.00 | — | 2.00 | 2.00 | — | 2.00 | 2.00 | — |
| Glycerin | 1.00 | 1.00 | 1.00 | 1.00 | 1.00 | 1.00 | 1.00 | 1.00 | 1.00 |
| Sodium Chloride | 1.00 | 1.00 | 1.00 | 1.00 | 1.00 | 1.00 | 1.00 | 1.00 | 1.00 |
| Pyranopyranone derivative | 0.10 | 0.20 | 0.30 | 0.40 | 0.50 | 0.60 | 0.70 | 0.80 | 1.00 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Citric Acid | Qs to | Qs to | Qs | Qs | Qs to | Qs to | Qs to | Qs to | Qs to |
| | pH 6 | pH 6 | to | to | pH 6 | pH 6 | pH 6 | pH 6 | pH 6 |
| | | | pH 6 | pH 6 | | | | | |

Clear shampoo

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Sodium Lauryl Sulfate | 1.00 | 1.00 | 1.00 | 1.00 | 1.00 | 1.00 | 1.00 | 1.00 | 1.00 |
| Sodium Polyoxyethylene | 12.00 | 12.00 | 12.00 | 12.00 | 12.00 | 12.00 | 12.00 | 12.00 | 12.00 |
| lauryl sulfate | | | | | | | | | |
| Lauroamidopropyl betaine | 1.00 | 1.00 | 1.00 | 1.00 | 1.00 | 1.00 | 1.00 | 1.00 | 1.00 |
| POE(16) lauryl ether | 1.00 | 1.00 | 1.00 | 1.00 | 1.00 | 1.00 | 1.00 | 1.00 | 1.00 |
| Glyceryl monoisodecyl | 0.50 | 0.50 | 0.50 | 0.50 | 0.50 | 0.50 | 0.50 | 0.50 | 0.50 |
| ether | | | | | | | | | |
| Cationized cellulose | 0.50 | 0.50 | 0.50 | 0.50 | 0.50 | 0.50 | 0.50 | 0.50 | 0.50 |
| Polyvinyl Alcohol | 0.20 | 0.20 | 0.20 | 0.20 | 0.20 | 0.20 | 0.20 | 0.20 | 0.20 |
| Glycerin | 10.00 | 10.00 | 10.00 | 10.00 | 10.00 | 10.00 | 10.00 | 10.00 | 10.00 |
| Tocopherol Acetate | 0.24 | 0.24 | 0.24 | 0.24 | 0.24 | 0.24 | 0.24 | 0.24 | 0.24 |
| Sodium Sulfate | 3.00 | 3.00 | 3.00 | 3.00 | 3.00 | 3.00 | 3.00 | 3.00 | 3.00 |
| Perfume | 0.10 | 0.10 | 0.10 | 0.10 | 0.10 | 0.10 | 0.10 | 0.10 | 0.10 |
| Sodium Hydroxide | q.s | q.s | q.s | q.s | q.s | q.s | q.s | q.s | q.s |
| | pH = 7 | pH = 7 | pH = 7 | pH = 7 | pH = 7 | pH = 7 | pH = 7 | pH = 7 | pH = 7 |
| Piroctone Olamine | 1.00 | 0.3 | 1.00 | — | 0.5 | — | — | 0.75 | — |
| Climbazole | — | 0.5 | — | 0.3 | 0.75 | 1.00 | — | — | 1.00 |
| Water | Qsp | Qsp | Qsp | Qsp | Qsp | Qsp | Qsp | Qsp | Qsp |
| | 100 | 100 | 100 | 100 | 100 | 100 | 100 | 100 | 100 |
| Pyranopyranone derivative | 0.10 | 0.20 | 0.30 | 0.40 | 0.50 | 0.60 | 0.70 | 0.80 | 1.00 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Sclerotium Gum | 0.10 | 0.10 | 0.10 | 0.10 | 0.10 | 0.10 | 0.10 | 0.10 | 0.10 |

Anti-Dandruff shampoo

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Water | Qs. 100 | Qs. 100 | Qs. 100 | Qs. 100 | Qs. 100 | Qs. 100 | Qs. 100 | Qs. 100 | Qs. 100 |
| Magnesium Aluminium | 2.00 | 2.00 | 2.00 | 2.00 | 2.00 | 2.00 | 2.00 | 2.00 | 2.00 |
| Silicate | | | | | | | | | |
| Ammonium Lauryl Sulfate | 20.00 | 20.00 | 20.00 | 20.00 | 20.00 | 20.00 | 20.00 | 20.00 | 20.00 |
| Ammonium Laureth Sulfate | 10.00 | 10.00 | 10.00 | 10.00 | 10.00 | 10.00 | 10.00 | 10.00 | 10.00 |
| Cocamidopropyl hydroxy- | 10.00 | 10.00 | 10.00 | 10.00 | 10.00 | 10.00 | 10.00 | 10.00 | 10.00 |
| sultaine | | | | | | | | | |
| Sodium Lauryol Sarcosinate | 3.00 | 3.00 | 3.00 | 3.00 | 3.00 | 3.00 | 3.00 | 3.00 | 3.00 |
| Di-PPG-2 Myreth-10 Adipate | 2.00 | 2.00 | 2.00 | 2.00 | 2.00 | 2.00 | 2.00 | 2.00 | 2.00 |
| PEG-150 Pentaerythrityl | 3.00 | 3.00 | 3.00 | 3.00 | 3.00 | 3.00 | 3.00 | 3.00 | 3.00 |
| Tetrastearate and PEG-6 | | | | | | | | | |
| Capriliy/Capric Glycerides | | | | | | | | | |
| and water | | | | | | | | | |
| Dimethicone | 0.20 | 0.20 | 0.20 | 0.20 | 0.20 | 0.20 | 0.20 | 0.20 | 0.20 |
| DMDM Hydantoin | 0.30 | 0.30 | 0.30 | 0.30 | 0.30 | 0.30 | 0.30 | 0.30 | 0.30 |
| Zinc Pyrithion, 48% | 2.00 | 2.00 | — | 2.00 | 2.00 | 2.00 | — | 2.00 | 2.00 |
| Piroctone Olamine | — | 0.3 | 1.00 | — | 0.5 | — | — | 0.75 | — |
| Climbazole | — | 0.5 | — | — | 0.75 | — | 0.80 | — | — |
| Citric Acid, 30% | qs. | qs. | qs. | qs. | qs. | qs. | qs. | qs. | qs. |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Shampoo

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Sodium Lauryl ether sulfate | 20.00 | 20.00 | 20.00 | 20.00 | 20.00 | 20.00 | 20.00 | 20.00 | 20.00 |
| Cocamidopropyl betaine | 7.00 | 7.00 | 7.00 | 7.00 | 7.00 | 7.00 | 7.00 | 7.00 | 7.00 |
| Polyquaternium-7 | 1.00 | 1.00 | 1.00 | 1.00 | 1.00 | 1.00 | 1.00 | 1.00 | 1.00 |
| Sodium chloride | 3.00 | 3.00 | 3.00 | 3.00 | 3.00 | 3.00 | 3.00 | 3.00 | 3.00 |
| Polysorbate 20 | 0.75 | 0.75 | 0.75 | 0.75 | 0.75 | 0.75 | 0.75 | 0.75 | 0.75 |
| Fragance | 0.20 | 0.20 | 0.20 | 0.20 | 0.20 | 0.20 | 0.20 | 0.20 | 0.20 |
| Pyranopyranone derivative | 0.00 | 0.10 | 0.20 | 0.30 | 0.40 | 0.50 | 0.25 | 0.50 | 0.40 |
| (Table 1) | | | | | | | | | |
| BIOCIDE/PRESERVATIVE | 0.50 | 0.40 | 0.30 | 0.20 | 0.10 | 0.00 | 0.25 | 0.50 | 0.40 |
| (Table 2) | | | | | | | | | |
| Lactic acid, 88%, to pH | qs | qs | qs | qs | qs | qs | qs | qs | qs |
| 5.00 | | | | | | | | | |
| FD&C blue #1 | 0.01 | 0.01 | 0.01 | 0.01 | 0.01 | 0.01 | 0.01 | 0.01 | 0.01 |
| Water | qs 100 | qs 100 | qs 100 | qs 100 | qs 100 | qs 100 | qs 100 | qs 100 | qs 100 |

Conditioning Shampoo

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Xanthan Gum | qs | qs | qs | qs | qs | qs | qs | qs | qs |
| Starch/acrylates/acrylamide co-polymer | 0.10 | 0.10 | 0.10 | 0.10 | 0.10 | 0.10 | 0.10 | 0.10 | 0.10 |
| Pyranopyranone derivative (Table 1) | 0.00 | 0.10 | 0.20 | 0.30 | 0.40 | 0.50 | 0.60 | 0.50 | 0.40 |
| BIOCIDE/PRESERVATIVE (Table 2) | 0.60 | 0.50 | 0.40 | 0.30 | 0.20 | 0.10 | 0.00 | 0.50 | 0.40 |
| TEA-lauryl sulfate | 25.00 | 25.00 | 25.00 | 25.00 | 25.00 | 25.00 | 25.00 | 25.00 | 25.00 |
| Lauramide DEQ | 3.50 | 3.50 | 3.50 | 3.50 | 3.50 | 3.50 | 3.50 | 3.50 | 3.50 |
| Mica and Titanium dioxode and | 0.05 | 0.05 | 0.05 | 0.05 | 0.05 | 0.05 | 0.05 | 0.05 | 0.05 |
| iron oxides | | | | | | | | | |
| Zinc Pyrithion, 48% | — | 1.00 | 0.50 | 0.80 | 1.00 | 2.00 | — | 2.00 | 2.00 |
| Piroctone Olamine | — | 0.3 | 1.00 | — | 0.5 | — | — | 0.75 | — |
| Climbazole | — | 0.25 | — | — | 0.75 | — | 1.00 | — | — |
| Panthenol | 0.80 | 0.80 | 0.80 | 0.80 | 0.80 | 0.80 | 0.80 | 0.80 | 0.80 |
| Green 3 FD&C, 0.5% | 0.02 | 0.02 | 0.02 | 0.02 | 0.02 | 0.02 | 0.02 | 0.02 | 0.02 |
| Fragance | 0.50 | 0.50 | 0.50 | 0.50 | 0.50 | 0.50 | 0.50 | 0.50 | 0.50 |
| Citric Acid, 20% | qs | qs | qs | qs | qs | qs | qs | qs | qs |
| Water | qs. 100 | qs. 100 | qs. 100 | qs. 100 | qs. 100 | qs. 100 | qs. 100 | qs. 100 | qs. 100 |

Pearlized Mild Body Wash

| Ingredients | Ex. 1 | Ex. 2 | Ex. 3 | Ex. 4 |
|---|---|---|---|---|
| Sodium Laureth | 15% | 15% | 15% | 15% |
| Sulfate | | | | |
| Cocoyl Isethionate | 4% | 5% | 3% | 6% |
| Sodium | 6% | 4% | 6% | 4% |
| Lauroamphoacetate | | | | |
| Sodium Xylene | 1% | 2% | 4% | 5% |
| Sulfonate | | | | |
| Sodium Methyl | 5% | 3% | 4% | 5% |
| Cocoyl Taurate | | | | |
| Acrylates | 1.5% | 1.5% | 1.5% | 1.5% |
| Crosspolymer | | | | |
| Dimethiconol and | 4% | 4% | 4% | 4% |
| TEA- | | | | |
| Dodecylbenzene- | | | | |
| sulfonate | | | | |
| Sodium | 2% | 2% | 2% | 2% |
| Cocoamphoacetate | | | | |
| Polyquaternium-7 | 2% | 2% | 2% | 2% |
| Sodium Hydroxide | 0.05% | 0.05% | 0.05% | 0.05% |
| (18% solution) | | | | |
| Guar Hydroxypropyl | 0.15% | 0.15% | 0.15% | 0.15% |
| trimonium Chloride | | | | |
| Citric acid | 0.05 | 0.05 | 0.05 | 0.05 |
| (50% solution) | | | | |
| Mica and Titanium | 0.2% | 0.2% | 0.2% | 0.2% |
| Dioxide | | | | |
| Perfume | 0.5% | 0.5% | 0.5% | 0.5% |
| Citric acid | 0.6% | 0.6% | 0.6% | 0.6% |
| (50% solution) | | | | |
| Pyranopyranone | 0.10% | 0.20% | 0.30% | 0.40% |
| derivative (Table 1) | | | | |
| BIOCIDE/ | 0.40% | 0.30% | 0.20% | 0.10% |
| PRESERVATIVE | | | | |
| (Table 2) | | | | |
| Sclerotium Gum | 0.50% | 2.50% | 5.00% | 10.00% |
| Water deionized | qs 100 | qs 100 | qs 100 | qs 100 |

Clear bath gel with suspended beads

| Ingredients | Ex. 1 | Ex. 2 | Ex. 3 | Ex. 4 |
|---|---|---|---|---|
| Sodium Laureth Sulfate | 30% | 30% | 30% | 30% |
| (28% solution) | | | | |
| Acrylates Crosspolymer | 10% | 10% | 10% | 10% |
| (30% solution) | | | | |
| Propylene Glycol | 2% | 2% | 2% | 2% |
| Sodium Hydroxide (18% solution) | 1.9% | 1.9% | 1.9% | 1.9% |
| Disodium EDTA | 0.1% | 0.1% | 0.1% | 0.1% |
| Benzophenone-4 | 0.02% | 0.02% | 0.02% | 0.02% |
| Cocamidopropyl Betaine | 4% | 4% | 4% | 4% |
| (35% solution) | | | | |
| Polysorbate 20 | 0.8% | 0.8% | 0.8% | 0.8% |
| White beads | 1% | 1% | 1% | 1% |
| Perfume | 0.6% | 0.6% | 0.6% | 0.6% |
| Citric acid (50% solution) | 0.6% | 0.6% | 0.6% | 0.6% |
| Preservative | 1% | 1% | 1% | 1% |
| Water deionized | Qsp 100 | Qsp 100 | Qsp 100 | Qsp 100 |
| Pyranopyranone derivative | 0.10% | 0.20% | 0.30% | 0.40% |
| (Table 1) | | | | |
| BIOCIDE/PRESERVATIVE | 0.40% | 0.30% | 0.20% | 0.10% |
| (Table 2) | | | | |

Feminine Hygiene cleansing formula

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Chamomile extract | 1.50 | 1.50 | 1.50 | 1.50 | 1.50 | 1.50 | 1.50 | 1.50 | 1.50 |
| Lactic acid (80% solution) | 0.01 | 0.01 | 0.01 | 0.01 | 0.01 | 0.01 | 0.01 | 0.01 | 0.01 |
| Disodium laureth sulfoccinate | 2.00 | 2.00 | 2.00 | 2.00 | 2.00 | 2.00 | 2.00 | 2.00 | 2.00 |
| PEG 150 disterarate | 1.00 | 1.00 | 1.00 | 1.00 | 1.00 | 1.00 | 1.00 | 1.00 | 1.00 |
| Water deioniz. | qs. 100 | qs. 100 | qs. 100 | qs. 100 | qs. 100 | qs. 100 | qs. 100 | qs. 100 | qs. 100 |
| Propylene glycol | 0.80 | 0.80 | 0.80 | 0.80 | 0.80 | 0.80 | 0.80 | 0.80 | 0.80 |
| Fragrance | qs | qs | qs | qs | qs | qs | qs | qs | qs |
| Sclerotium Gum | 0.10 | 0.10 | 0.10 | 0.10 | 0.10 | 0.10 | 0.10 | 0.10 | 0.10 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Feminine Hygiene Wash

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Water | qs to 100 | qs to 100 | qs to 100 | qs to 100 | qs to 100 | qs to 100 | qs to 100 | qs to 100 | qs to 100 |
| Pyranopyranone derivative | 0.10 | 0.20 | 0.30 | 0.40 | 0.50 | 0.50 | 0.50 | 0.50 | 0.50 |
| (Table 1) | | | | | | | | | |
| BIOCIDE/PRESERVATIVE | 0.50 | 0.50 | 0.50 | 0.50 | 0.50 | 0.10 | 0.20 | 0 | 0 |
| (Table 2) | | | | | | | | | |
| Triclosan | 0.10 | 0.30 | 0 | 0 | 0.10 | 0 | 0 | 0 | 0 |
| Fragrance | qs | qs | qs | qs | qs | qs | qs | qs | qs |
| Sodium Laureth Sulfate | 25.00 | 25.00 | 25.00 | 25.00 | 25.00 | 25.00 | 25.00 | 25.00 | 25.00 |
| Cocamidopropyl Betaine | 5.00 | 5.00 | 5.00 | 5.00 | 5.00 | 5.00 | 5.00 | 5.00 | 5.00 |
| (and) Glyceryl Laurate | | | | | | | | | |
| Lactic Acid | 0.50 | 0.50 | 0.50 | 0.50 | 0.50 | 0.50 | 0.50 | 0.50 | 0.50 |
| Sodium Chloride | 1.00 | 1.00 | 1.00 | 1.00 | 1.00 | 1.00 | 1.00 | 1.00 | 1.00 |
| Allantoin | 0.10 | 0.10 | 0.10 | 0.10 | 0.10 | 0.10 | 0.10 | 0.10 | 0.10 |
| Water (and) Sodium | ad pH | ad pH | ad pH | ad pH | ad pH | ad pH | ad pH | ad pH | ad pH |
| Hydroxide | 5.0 | 5.0 | 5.0 | 5.0 | 5.0 | 5.0 | 5.0 | 5.0 | 5.0 |

Mild Feminine Hygiene Wash

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Water | qs to 100 | qs to 100 | qs to 100 | qs to 100 | qs to 100 | qs to 100 | qs to 100 | qs to 100 | qs to 100 |
| Sodium Laureth Sulfate | 20.00 | 20.00 | 20.00 | 20.00 | 20.00 | 20.00 | 20.00 | 20.00 | 20.00 |
| Cocamidopropyl Betaine | 7.00 | 7.00 | 7.00 | 7.00 | 7.00 | 7.00 | 7.00 | 7.00 | 7.00 |
| Laureth 02 | 1.25 | 1.25 | 1.25 | 1.25 | 1.25 | 1.25 | 1.25 | 1.25 | 1.25 |
| Sodium Laureth-11 | 1.25 | 1.25 | 1.25 | 1.25 | 1.25 | 1.25 | 1.25 | 1.25 | 1.25 |
| Carboxylate | | | | | | | | | |
| PEG-7 Glyceryl Cocoate | 1.00 | 1.00 | 1.00 | 1.00 | 1.00 | 1.00 | 1.00 | 1.00 | 1.00 |
| Propylene Glycol | 3.00 | 3.00 | 3.00 | 3.00 | 3.00 | 3.00 | 3.00 | 3.00 | 3.00 |
| Pyranopyranone derivative | 0.10 | 0.20 | 0.30 | 0.40 | 0.50 | 0.50 | 0.50 | 0.50 | 0.50 |
| (Table 1) | | | | | | | | | |
| BIOCIDE/PRESERVATIVE | 0.50 | 0 | 0.50 | 0.50 | 0 | 0.10 | 0.20 | 0.30 | 0.40 |
| (Table 2) | | | | | | | | | |
| Fragrance | qs | qs | qs | qs | qs | qs | qs | qs | qs |
| Lactic Acid | 0.50 | 0.50 | 0.50 | 0.50 | 0.50 | 0.50 | 0.50 | 0.50 | 0.50 |
| Sodium Chloride | 1.00 | 1.00 | 1.00 | 1.00 | 1.00 | 1.00 | 1.00 | 1.00 | 1.00 |
| Water (and) Sodium | ad pH | ad pH | ad pH | ad pH | ad pH | ad pH | ad pH | ad pH | ad pH |
| Hydroxide | 5.0 | 5.0 | 5.0 | 5.0 | 5.0 | 5.0 | 5.0 | 5.0 | 5.0 |

Personal Lubricant

| Ingredients | Ex. 1 | Ex. 2 | Ex. 3 | Ex. 4 |
|---|---|---|---|---|
| Lubricating | 5% | 10% | 8% | 15% |
| agents | | | | |
| Lanolin | 8% | 8% | 8% | 8% |
| anhydrous | | | | |
| Sweet almond | 5% | 5% | 5% | 5% |
| oil | | | | |
| Coconut oil | 10.75% | 10.75% | 10.75% | 10.75% |
| Cetyl alcohol | 0.5% | 0.5% | 0.5% | 0.5% |
| Mineral oil | 1% | 1% | 1% | 1% |
| Preservative | 0.3% | 0.3% | 0.3% | 0.3% |
| Water deioniz. | Qsp | Qsp | Qsp | Qsp |
| Sodium lauryl | 1.5% | 1.5% | 1.5% | 1.5% |
| sulfate | | | | |
| Propylene | 3% | 3% | 3% | 3% |
| glycol | | | | |
| Aloe vera gel | 9% | 9% | 9% | 9% |
| Sorbitol | 1% | 1% | 1% | 1% |
| Allantoin | 2.5% | 2.5% | 2.5% | 2.5% |
| Polyethylene | 10% | 13% | 12% | 15% |
| oxides of | | | | |
| MW < 100 000 | | | | |
| Xanthan gum | 0.5% | 0.5% | 0.5% | 0.5% |
| Coconut flavor | 0.9% | 0.9% | 0.9% | 0.9% |
| Pyranopyranone | 0.10% | 0.20% | 0.30% | 0.40% |
| derivative | | | | |
| (Table 1) | | | | |
| BIOCIDE/ | 0.40% | 0.30% | 0.20% | 0.10% |
| PRESERV- | | | | |
| ATIVE | | | | |
| (Table 2) | | | | |
| Sclerotium | 0.50% | 2.50% | 5.00% | 10.00% |
| Gum | | | | |

Liquid Dentrifrice formulation

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Sodium Lauryl Sulfate | 1.50 | 1.50 | 1.50 | 1.50 | 1.50 | 1.50 | 1.50 | 1.50 | 1.50 |
| Triclosan | 0.10 | 0.30 | 0 | 0 | 0.15 | 0.05 | 0 | 0 | 0.05 |
| Benzalkonium chloride | 0 | 0 | 0 | 0.10 | 0 | 0 | 0 | 0 | 0.03 |
| (or bromide) | | | | | | | | | |
| Cetrimonium chloride | 0 | 0 | 0.10 | 0 | 0 | 0.03 | 0 | 0 | 0 |
| (or bromide) | | | | | | | | | |
| Pyranopyranone derivative | 0.01 | 0.02 | 0.05 | 0.08 | 0.10 | 0.15 | 0.20 | 0.30 | 0.50 |
| (Table 1) | | | | | | | | | |
| Propylene Glycol | 0.50 | 0.50 | 0.50 | 0.50 | 0.50 | 0.50 | 0.50 | 0.50 | 0.50 |
| Glycerin | 23.40 | 23.40 | 23.40 | 23.40 | 23.40 | 23.40 | 23.40 | 23.40 | 23.40 |
| Sodium Fluoride | 0.24 | 0.24 | 0.24 | 0.24 | 0.24 | 0.24 | 0.24 | 0.24 | 0.24 |
| Sorbitol 70% | 20.00 | 20.00 | 20.00 | 20.00 | 20.00 | 20.00 | 20.00 | 20.00 | 20.00 |
| BIOCIDE/PRESERVATIVE | 0.01 | 0.02 | 0.03 | 0.04 | 0.05 | 0.06 | 0.07 | 0.10 | 0.15 |
| (Table 2) | | | | | | | | | |
| Water | Qs to 100 | Qs to 100 | Qs to 100 | Qs to 100 | Qs to 100 | Qs to 100 | Qs to 100 | Qs to 100 | Qs to 100 |

Mouth Rinse formulation

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Sodium Lauryl Sulfate | 0.60 | 0.60 | 0.60 | 0.60 | 0.60 | 0.60 | 0.60 | 0.60 | 0.60 |
| Ethanol | 15.00 | 15.00 | 15.00 | 15.00 | 15.00 | 15.00 | 15.00 | 15.00 | 15.00 |
| Pyranopyranone derivative | 0.01 | 0.02 | 0.05 | 0.08 | 0.10 | 0.15 | 0.20 | 0.30 | 0.50 |
| (Table 1) | | | | | | | | | |
| Triclosan | 0.05 | 0 | 0 | 0 | 0.10 | 0 | 0.05 | 0 | 0 |
| Benzalkonium chloride | 0 | 0.05 | 0.10 | 0 | 0 | 0 | 0 | 0 | 0.01 |
| (or bromide) | | | | | | | | | |
| Cetrimonium chloride | 0.05 | 0 | 0 | 0.05 | 0.05 | 0.10 | 0 | 0 | 0 |
| (or bromide) | | | | | | | | | |
| Propylene Glycol | 7.00 | 7.00 | 7.00 | 7.00 | 7.00 | 7.00 | 7.00 | 7.00 | 7.00 |
| Glycerin | 11.70 | 11.70 | 11.70 | 11.70 | 11.70 | 11.70 | 11.70 | 11.70 | 11.70 |
| Sorbitol 70% | 10.00 | 10.00 | 10.00 | 10.00 | 10.00 | 10.00 | 10.00 | 10.00 | 10.00 |
| Sodium Saccharine | 0.03 | 0.03 | 0.03 | 0.03 | 0.03 | 0.03 | 0.03 | 0.03 | 0.03 |
| BIOCIDE/PRESERVATIVE | 0.01 | 0.02 | 0.03 | 0.04 | 0.05 | 0.06 | 0.07 | 0.10 | 0.15 |
| (Table 2) | | | | | | | | | |
| Water | Qs to 100 | Qs to 100 | Qs to 100 | Qs to 100 | Qs to 100 | Qs to 100 | Qs to 100 | Qs to 100 | Qs to 100 |

Mouthwash

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Pyranopyranone derivative | 0.05 | 0.05 | 0.05 | 0.05 | 0.05 | 0.05 | 0.10 | 0.05 | 0.20 |
| (Table 1) | | | | | | | | | |
| Glycerin | 10.00 | 10.00 | 10.00 | 10.00 | 10.00 | 10.00 | 10.00 | 10.00 | 10.00 |
| Triclosan | 0.10 | 0 | 0 | 0 | 0 | 0 | 0 | 0 | 0 |
| SD Alcohol 39-C | 4.00 | 4.00 | 4.00 | 4.00 | 4.00 | 4.00 | 4.00 | 4.00 | 4.00 |
| Flavour | qs | qs | qs | qs | qs | qs | qs | qs | qs |
| Poloxamer 407 | 2.00 | 2.00 | 2.00 | 2.00 | 2.00 | 2.00 | 2.00 | 2.00 | 2.00 |
| Sodium Saccharin | 0.01 | 0.01 | 0.01 | 0.01 | 0.01 | 0.01 | 0.01 | 0.01 | 0.01 |
| Sodium Fluoride | 0.05 | 0.05 | 0.05 | 0.05 | 0.05 | 0.05 | 0.05 | 0.05 | 0.05 |
| Sodium Benzoate | 0.10 | 0.10 | 0.10 | 0.10 | 0.10 | 0.10 | 0.10 | 0.10 | 0.10 |
| BIOCIDE/PRESERVATIVE | 0.01 | 0.02 | 0.03 | 0.04 | 0.05 | 0.06 | 0.07 | 0.10 | 0.15 |
| (Table 2) | | | | | | | | | |
| Water | qs to 100 | qs to 100 | qs to 100 | qs to 100 | qs to 100 | qs to 100 | qs to 100 | qs to 100 | qs to 100 |

Mild transparent Toothgel

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Sorbitol | 23.50 | 23.50 | 23.50 | 23.50 | 23.50 | 23.50 | 23.50 | 23.50 | 23.50 |
| Sodium Saccharin | 0.20 | 0.20 | 0.20 | 0.20 | 0.20 | 0.20 | 0.20 | 0.20 | 0.20 |
| Sodium Citrate | 0.20 | 0.20 | 0.20 | 0.20 | 0.20 | 0.20 | 0.20 | 0.20 | 0.20 |
| Betaine | 2.00 | 2.00 | 2.00 | 2.00 | 2.00 | 2.00 | 2.00 | 2.00 | 2.00 |
| Triclosan | 0.05 | 0.10 | 0 | 0 | 0 | 0 | 0 | 0 | 0 |
| Glycerin | 25.00 | 25.00 | 25.00 | 25.00 | 25.00 | 25.00 | 25.00 | 25.00 | 25.00 |
| Water | qs to 100 | qs to 100 | qs to 100 | qs to 100 | qs to 100 | qs to 100 | qs to 100 | qs to 100 | qs to 100 |
| PEG-8 | 1.75 | 1.75 | 1.75 | 1.75 | 1.75 | 1.75 | 1.75 | 1.75 | 1.75 |
| Gellan Gum | 0.25 | 0.25 | 0.25 | 0.25 | 0.25 | 0.25 | 0.25 | 0.25 | 0.25 |
| Xanthan Gum | 0.25 | 0.25 | 0.25 | 0.25 | 0.25 | 0.25 | 0.25 | 0.25 | 0.25 |
| Hydrated Silica | 15.00 | 15.00 | 15.00 | 15.00 | 15.00 | 15.00 | 15.00 | 15.00 | 15.00 |
| FD&C Blue No. 1 | 0.10 | 0.10 | 0.10 | 0.10 | 0.10 | 0.10 | 0.10 | 0.10 | 0.10 |
| Propylene Glycol (and) | 3.00 | 3.00 | 3.00 | 3.00 | 3.00 | 3.00 | 3.00 | 3.00 | 3.00 |
| Olaflur | | | | | | | | | |
| Aroma | 0.75 | 0.75 | 0.75 | 0.75 | 0.75 | 0.75 | 0.75 | 0.75 | 0.75 |
| Pyranopyranone derivative | 0.05 | 0.10 | 0.20 | 0.30 | 0.40 | 0.50 | 0.60 | 0.70 | 0.80 |
| (Table 1) | | | | | | | | | |
| BIOCIDE/PRESERVATIVE | 0.01 | 0.02 | 0.03 | 0.04 | 0.05 | 0.06 | 0.07 | 0.10 | 0.15 |
| (Table 2) | | | | | | | | | |
| Sodium Lauroyl Sarcosinate | 6.00 | 6.00 | 6.00 | 6.00 | 6.00 | 6.00 | 6.00 | 6.00 | 6.00 |

Opaque silica-based dentifrice

| Ingredients | a | b | c | d |
|---|---|---|---|---|
| Sodium Lauryl Sulfate | 1.50% | 2.00% | 1.00% | 2.50% |
| Sodium Carboxymethyl cellulose | 0.70% | 1.00% | 0.90% | 0.80% |
| or Xanthan gum | | | | |
| Sorbitol (70%) | 45% | 45% | 45% | 45% |
| Abrasive silica | 10% | 10% | 10% | 10% |
| Thickening silica | 8-10% | 8-10% | 8-10% | 8-10% |
| PEG 1500 | 5% | 5% | 5% | 5% |
| Titanium Dioxide (opacifier) | 1% | 1% | 1% | 1% |
| Sodium Saccharinate | qs | qs | qs | qs |
| Flavouring agents | <1.5% | <1.5% | <1.5% | <1.5% |
| Colorants | qs | qs | qs | qs |
| Pyranopyranone derivative | 0.10 | 0.20 | 0.30 | 0.40 |
| (Table 1) | | | | |
| BIOCIDE/PRESERVATIVE | 0.40 | 0.30 | 0.20 | 0.10 |
| (Table 2) | | | | |
| Triclosan | 0 | 0.05 | 0 | 0 |
| Water deionized | Qsp 100 | Qsp 100 | Qsp 100 | Qsp 100 |
| Vitamins A, C or E | 0.80% | 0.50% | — | 1.00% |
| (free or as derivative) | | | | |

Deodorant Stick

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Propylene Glycol | 18.10 | 18.10 | 18.10 | 18.10 | 18.10 | 18.10 | 18.10 | 18.10 | 18.10 |
| PPG-3 Myristyl Ether | Q | 46.70 | 46.70 | 46.70 | 46.70 | 46.70 | 46.70 | 46.70 | 46.70 |
| Cyclomethicone | 28.90 | 28.90 | 28.90 | 28.90 | 28.90 | 28.90 | 28.90 | 28.90 | 28.90 |
| Sodium Stearate | 6.00 | 6.00 | 6.00 | 6.00 | 6.00 | 6.00 | 6.00 | 6.00 | 6.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Deodorant Pump spray

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| PEG-40 Hydrogenated | 4.50 | 4.50 | 4.50 | 4.50 | 4.50 | 4.50 | 4.50 | 4.50 | 4.50 |
| castor Oil | | | | | | | | | |
| PEG-08 | 1.00 | 1.00 | 1.00 | 1.00 | 1.00 | 1.00 | 1.00 | 1.00 | 1.00 |
| Glyceryl Laurate | 0.50 | 0.50 | 0.50 | 0.50 | 0.50 | 0.50 | 0.50 | 0.50 | 0.50 |
| Cetearyl Octanoate (and) | 1.00 | 1.00 | 1.00 | 1.00 | 1.00 | 1.00 | 1.00 | 1.00 | 1.00 |
| Isopropyl Myristate | | | | | | | | | |
| Alcohol | 30.00 | 30.00 | 30.00 | 30.00 | 30.00 | 30.00 | 30.00 | 30.00 | 30.00 |
| Water | Qs to 100 | Qs to 101 | Qs to 102 | Qs to 103 | Qs to 104 | Qs to 105 | Qs to 106 | Qs to 107 | Qs to 108 |
| Aluminum Chlorohydrate | 30.00 | 31.00 | 32.00 | 33.00 | 34.00 | 35.00 | 36.00 | 37.00 | 38.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

O/W-Deodorant

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Gylceryl Stearate (and) | 3.00 | 3.00 | 3.00 | 3.00 | 3.00 | 3.00 | 3.00 | 3.00 | 3.00 |
| Ceteth-20 | | | | | | | | | |
| Cetearyl Ethylhexanoate | 3.00 | 3.00 | 3.00 | 3.00 | 3.00 | 3.00 | 3.00 | 3.00 | 3.00 |
| Stearyl Alcohol | 1.00 | 1.00 | 1.00 | 1.00 | 1.00 | 1.00 | 1.00 | 1.00 | 1.00 |
| Dicaprylyl Ether | 6.00 | 6.00 | 6.00 | 6.00 | 6.00 | 6.00 | 6.00 | 6.00 | 6.00 |
| Dimethicone | 0.50 | 0.50 | 0.50 | 0.50 | 0.50 | 0.50 | 0.50 | 0.50 | 0.50 |
| Water | Qsp to | Qsp to | Qsp to | Qsp to | Qsp to | Qsp to | Qsp to | Qsp to | Qsp to |
| | 100 | 100 | 100 | 100 | 100 | 100 | 100 | 100 | 100 |
| Farnesol | 0.05 | 0.10 | 0 | 0 | 0 | 0 | 0.25 | 0 | 0 |
| Sodium Polyacrylate | 0.10 | 0.10 | 0.10 | 0.10 | 0.10 | 0.10 | 0.10 | 0.10 | 0.10 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Antiperspirant roll-on

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| PEG 60 Hydrogenated | 2.50 | 2.50 | 2.50 | 2.50 | 2.50 | 2.50 | 2.50 | 2.50 | 2.50 |
| Castor Oil | | | | | | | | | |
| PEG-6 Caprylic Capric | 2.00 | 2.00 | 2.00 | 2.00 | 2.00 | 2.00 | 2.00 | 2.00 | 2.00 |
| Glyceride | | | | | | | | | |
| Hydroxyethylcellulose | 0.50 | 0.50 | 0.50 | 0.50 | 0.50 | 0.50 | 0.50 | 0.50 | 0.50 |
| Water | Qs to 100 | Qs to 100 | Qs to 100 | Qs to 100 | Qs to 100 | Qs to 100 | Qs to 100 | Qs to 100 | Qs to 100 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Aluminum Chlorohydrate | 30.00 | 30.00 | 30.00 | 30.00 | 30.00 | 30.00 | 30.00 | 30.00 | 30.00 |
| Glycerin | 2.00 | 2.00 | 2.00 | 2.00 | 2.00 | 2.00 | 2.00 | 2.00 | 2.00 |
| Propylene Glycol | 2.00 | 2.00 | 2.00 | 2.00 | 2.00 | 2.00 | 2.00 | 2.00 | 2.00 |

Alcohol-free Deodorant roll-on

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| PEG 60 Hydrogenated Castor | 3.00 | 3.00 | 3.00 | 3.00 | 3.00 | 3.00 | 3.00 | 3.00 | 3.00 |
| Oil | | | | | | | | | |
| PEG-6 Caprylic Capric | 3.00 | 3.00 | 3.00 | 3.00 | 3.00 | 3.00 | 3.00 | 3.00 | 3.00 |
| Glyceride | | | | | | | | | |
| Propylene Glycol | 4.00 | 4.00 | 4.00 | 4.00 | 4.00 | 4.00 | 4.00 | 4.00 | 4.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| Steareth-10 allyl | 5.00 | 5.00 | 5.00 | 5.00 | 5.00 | 5.00 | 5.00 | 5.00 | 5.00 |
| ether/acrylates copolymer | | | | | | | | | |
| Ethanolamine | qs | qs | qs | qs | qs | qs | qs | qs | qs |
| Water | Qs to 100 | Qs to 100 | Qs to 100 | Qs to 100 | Qs to 100 | Qs to 100 | Qs to 100 | Qs to 100 | Qs to 100 |

Formulation for Tissues, Moisturizing Face Mask

| | A | B | C | D | E | F | G | H | I |
|---|---|---|---|---|---|---|---|---|---|
| INCI-Name | % w/w | % w/w | % w/w | % w/w | % w/w | % w/w | % w/w | % w/w | % w/w |
| Water | qs to 100 | qs to 100 | qs to 100 | qs to 100 | qs to 100 | qs to 100 | qs to 100 | qs to 100 | qs to 100 |
| Sclerotium Gum | 10.0 | 10.0 | 10.0 | 10.0 | 10.0 | 10.0 | 10.0 | 10.0 | 10.0 |
| Glycerin | 4.00 | 4.00 | 4.00 | 4.00 | 4.00 | 4.00 | 4.00 | 4.00 | 4.00 |
| Butylene Glycol | 2.00 | 2.00 | 2.00 | 2.00 | 2.00 | 2.00 | 2.00 | 2.00 | 2.00 |
| Allantoin | 0.10 | 0.10 | 0.10 | 0.10 | 0.10 | 0.10 | 0.10 | 0.10 | 0.10 |
| Xanthan Gum | 0.10 | 0.10 | 0.10 | 0.10 | 0.10 | 0.10 | 0.10 | 0.10 | 0.10 |
| Aqua (and) Tocopheryl | 3.00 | 3.00 | 3.00 | 3.00 | 3.00 | 3.00 | 3.00 | 3.00 | 3.00 |
| Acetate (and) Caprylic/Caric | | | | | | | | | |
| Triglyceride (and) Poly- | | | | | | | | | |
| sorbate 80 (and) Lecithin | | | | | | | | | |
| Aqua (and) Retinyl Palmitate | 1.00 | 1.00 | 1.00 | 1.00 | 1.00 | 1.00 | 1.00 | 1.00 | 1.00 |
| (and) Caprylic/Capric | | | | | | | | | |
| Triglyceride (and) Polysorbate | | | | | | | | | |
| 80 (and) Lecithin | | | | | | | | | |
| (and) Benzalkonium Chloride | | | | | | | | | |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Antibacterial Shaving Foam

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Stearic Acid | 7.00 | 7.00 | 7.00 | 7.00 | 7.00 | 7.00 | 7.00 | 7.00 | 7.00 |
| Lauric Acid | 2.00 | 2.00 | 2.00 | 2.00 | 2.00 | 2.00 | 2.00 | 2.00 | 2.00 |
| Triethanolamine | 7.00 | 7.00 | 7.00 | 7.00 | 7.00 | 7.00 | 7.00 | 7.00 | 7.00 |
| Glycerin | 9.00 | 9.00 | 9.00 | 9.00 | 9.00 | 9.00 | 9.00 | 9.00 | 9.00 |
| PEG-14 Dimethicone | 2.00 | 2.00 | 2.00 | 2.00 | 2.00 | 2.00 | 2.00 | 2.00 | 2.00 |
| Perfume | qs | qs | qs | qs | qs | qs | qs | qs | qs |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Pyranopyranone derivative | 0.10 | 0.25 | 0.50 | 0.10 | 0.25 | 0.50 | 0.10 | 0.25 | 0.50 |
| (Table 1) | | | | | | | | | |
| Water | qs to 100 | qs to 100 | qs to 100 | qs to 100 | qs to 100 | qs to 100 | qs to 100 | qs to 100 | qs to 100 |

Foot Cream

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Polyglyceryl-3 | 4.00 | 4.00 | 4.00 | 4.00 | 4.00 | 4.00 | 4.00 | 4.00 | 4.00 |
| Diisostearate | | | | | | | | | |
| Beeswax | 3.00 | 3.00 | 3.00 | 3.00 | 3.00 | 3.00 | 3.00 | 3.00 | 3.00 |
| Glyceryl Oleate | 2.00 | 2.00 | 2.00 | 2.00 | 2.00 | 2.00 | 2.00 | 2.00 | 2.00 |
| Zinc Stearate | 2.00 | 2.00 | 2.00 | 2.00 | 2.00 | 2.00 | 2.00 | 2.00 | 2.00 |
| Hexyldecanol | 10.00 | 10.00 | 10.00 | 10.00 | 10.00 | 10.00 | 10.00 | 10.00 | 10.00 |
| C12-15 Alkyl Benzoate | 10.00 | 10.00 | 10.00 | 10.00 | 10.00 | 10.00 | 10.00 | 10.00 | 10.00 |
| Glycerin | 4.00 | 4.00 | 4.00 | 4.00 | 4.00 | 4.00 | 4.00 | 4.00 | 4.00 |
| Magnesium Sulfate | 1.00 | 1.00 | 1.00 | 1.00 | 1.00 | 1.00 | 1.00 | 1.00 | 1.00 |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Sodium Hydroxide | qs | qs | qs | qs | qs | qs | qs | qs | qs |
| Water | Qs to 100 | Qs to 100 | Qs to 100 | Qs to 100 | Qs to 100 | Qs to 100 | Qs to 100 | Qs to 100 | Qs to 100 |

Deodorant and Antiseptic Foot Cream

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Polyglyceryl-3 | 4.00 | 4.00 | 4.00 | 4.00 | 4.00 | 4.00 | 4.00 | 4.00 | 4.00 |
| Diisostearate | | | | | | | | | |
| Glyceryl Oleate | 2.00 | 2.00 | 2.00 | 2.00 | 2.00 | 2.00 | 2.00 | 2.00 | 2.00 |
| Zinc Stearate | 2.00 | 2.00 | 2.00 | 2.00 | 2.00 | 2.00 | 2.00 | 2.00 | 2.00 |
| Caprylic/Capric Triglyceride | 10.00 | 10.00 | 10.00 | 10.00 | 10.00 | 10.00 | 10.00 | 10.00 | 10.00 |
| Hexyldecanol | 10.00 | 10.00 | 10.00 | 10.00 | 10.00 | 10.00 | 10.00 | 10.00 | 10.00 |
| Beeswax | 3.00 | 3.00 | 3.00 | 3.00 | 3.00 | 3.00 | 3.00 | 3.00 | 3.00 |
| Pyranopyranone derivative | 0.30 | 0.30 | 0.30 | 0.30 | 0.10 | 0.25 | 0.20 | 0.30 | 0.30 |
| (Table 1) | | | | | | | | | |
| BIOCIDE/PRESERVATIVE | 0.10 | 0.25 | 0.20 | 0.30 | 0.30 | 0.30 | 0.30 | 0.30 | 0.00 |
| (Table 2) | | | | | | | | | |
| Glycerin | 4.00 | 4.00 | 4.00 | 4.00 | 4.00 | 4.00 | 4.00 | 4.00 | 4.00 |
| Water | qs to 100 | qs to 100 | qs to 100 | qs to 100 | qs to 100 | qs to 100 | qs to 100 | qs to 100 | qs to 100 |
| Magnesium Sulfate | 1.00 | 1.00 | 1.00 | 1.00 | 1.00 | 1.00 | 1.00 | 1.00 | 1.00 |

Deodorant Foot Powder

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Pyranopyranone derivative | 0.1 | 0.2 | 0.3 | 0.4 | 0.5 | 0.6 | 0.75 | 1 | 1.5 |
| (Table 1) | | | | | | | | | |
| Talc | qs to 100 | qs to 100 | qs to 100 | qs to 100 | qs to 100 | qs to 100 | qs to 100 | qs to 100 | qs to 100 |
| Magnesium Carbonate | 10.00 | 10.00 | 10.00 | 10.00 | 10.00 | 10.00 | 10.00 | 10.00 | 10.00 |
| Rice Starch | 5.00 | 5.00 | 5.00 | 5.00 | 5.00 | 5.00 | 5.00 | 5.00 | 5.00 |
| Zinc Undecylinate | 10.00 | 10.00 | 10.00 | 10.00 | 10.00 | 10.00 | 10.00 | 10.00 | 10.00 |
| Potassium Sulfate | 4.00 | 4.00 | 4.00 | 4.00 | 4.00 | 4.00 | 4.00 | 4.00 | 4.00 |
| Allantoin | 0.50 | 0.50 | 0.50 | 0.50 | 0.50 | 0.50 | 0.50 | 0.50 | 0.50 |
| Pinus Palustris Oil | 0.50 | 0.50 | 0.50 | 0.50 | 0.50 | 0.50 | 0.50 | 0.50 | 0.50 |
| BIOCIDE/PRESERVATIVE | 0.10 | 0.25 | 0.20 | 0.30 | 0.30 | 0.30 | 0.30 | 0.30 | 0.00 |
| (Table 2) | | | | | | | | | |

Deodorant Footspray

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Pyranopyranone derivative | 0.1 | 0.2 | 0.3 | 0.4 | 0.5 | 0.6 | 0.75 | 1 | 1.5 |
| (Table 1) | | | | | | | | | |
| PEG-40 Hydrogenated | 4.50 | 4.50 | 4.50 | 4.50 | 4.50 | 4.50 | 4.50 | 4.50 | 4.50 |
| Castor Oil | | | | | | | | | |
| PEG-8 | 1.00 | 1.00 | 1.00 | 1.00 | 1.00 | 1.00 | 1.00 | 1.00 | 1.00 |
| Glyceryl Laurate | 0.50 | 0.50 | 0.50 | 0.50 | 0.50 | 0.50 | 0.50 | 0.50 | 0.50 |
| Cetearyl Ethylhexanoate | 1.00 | 1.00 | 1.00 | 1.00 | 1.00 | 1.00 | 1.00 | 1.00 | 1.00 |
| (and) Isopropyl Myristate | | | | | | | | | |
| SD Alcohol 39-C | 30.00 | 30.00 | 30.00 | 30.00 | 30.00 | 30.00 | 30.00 | 30.00 | 30.00 |
| Perfume | qs | qs | qs | qs | qs | qs | qs | qs | qs |
| Water | qs to 100 | qs to 100 | qs to 100 | qs to 100 | qs to 100 | qs to 100 | qs to 100 | qs to 100 | qs to 100 |
| BIOCIDE/PRESERVATIVE | 0.10 | 0.25 | 0.20 | 0.30 | 0.30 | 0.30 | 0.30 | 0.30 | 0.40 |
| (Table 2) | | | | | | | | | |

Non-aqueous antiperspirant spray

| | A | B | C | D | E | F | G | H | I |
|---|---|---|---|---|---|---|---|---|---|
| | % | % | % | % | % | % | % | % | % |
| Silicone oil DC ® 245 | 28 | 28 | 23 | 23 | 23 | 38 | 42 | 47 | 31 |
| Eutanol ® G 16 | 10 | — | — | 15 | 10 | — | 10 | — | 10 |
| Cetiol ® OE | — | 10 | 15 | — | — | — | — | — | — |
| Ucon Fluid ® AP | 5 | 5 | 5 | 5 | 5 | 5 | 5 | 5 | 5 |
| Cutina ® HR | 6 | 6 | 6 | 6 | 6 | 6 | 2 | 5 | 6 |
| Lorol ® C 18 | 20 | 20 | 20 | — | 20 | 20 | — | — | 20 |
| Lanette ® O | — | — | — | 20 | — | — | 10 | 12 | — |
| Eumulgin ® B 3 | 3 | 3 | 3 | 3 | 3 | 3 | 3 | 3 | — |
| Cutina ® E 24 PF | — | — | — | — | 5 | — | — | — | — |
| Aluminium chlorohydrate | 20 | 20 | 20 | 20 | 20 | 20 | 20 | — | — |
| Talcum | 7.995 | 7.9 | 7.9 | 7.9 | 7.5 | 7.5 | 7.995 | 27.9 | 27.5 |
| Pyranopyranone derivative | 0.005 | 0.01 | 0.01 | 0.005 | 0.10 | 0.01 | 0.05 | 0.50 | 0.25 |
| (Table 1) | | | | | | | | | |
| BIOCIDE/PRESERVATIVE | 0.10 | 0.25 | 0.50 | 1.00 | 0.10 | 0 | 0 | 0 | 0 |
| (Table 2) | | | | | | | | | |

Deodorant Microemulsion Spray

| | A | B | C | D | E | F | G | H | I |
|---|---|---|---|---|---|---|---|---|---|
| | % | % | % | % | % | % | % | % | % |
| Plantaren ® 1200 | 1.71 | 1.71 | — | 1.71 | 1.71 | — | 1.71 | 1.71 | 1.71 |
| Plantaren ® 2000 | 1.14 | 1.39 | 2.40 | 1.14 | 1.39 | 2.40 | 1.14 | 1.39 | 1.39 |
| Glycerol monooleate | 0.71 | 0.71 | — | 0.71 | 0.71 | — | 0.71 | 0.71 | 0.71 |
| Dioctyl ether | 4.00 | 4.00 | 0.09 | 4.00 | 4.00 | 0.09 | 4.00 | 4.00 | 4.00 |
| Octyl dodecanol | 1.00 | 1.00 | 0.02 | 1.00 | 1.00 | 0.02 | 1.00 | 1.00 | 1.00 |
| Perfume oil | 1.00 | 1.00 | 1.00 | 1.00 | 1.00 | 1.00 | 1.00 | 1.00 | 1.00 |
| Aluminium chlorohydrate | 8.00 | 5.00 | 5.00 | — | — | — | 8.00 | 5.00 | 5.00 |
| 1,2-Propylene glycol | 5.00 | 5.00 | — | 5.00 | 5.00 | — | 5.00 | 5.00 | 5.00 |
| Glycerol | — | — | 5.00 | — | — | 5.00 | — | — | — |
| Tocopheryl acetate | 0.6 | 0.6 | 0.6 | 0.6 | 0.6 | 0.6 | 0.6 | 0.6 | 0.6 |
| Pyranopyranone derivative | 0.05 | 0.01 | 0.10 | 0.20 | 0.30 | 0.50 | 0.15 | 0.25 | 1.00 |
| (Table 1) | | | | | | | | | |
| BIOCIDE/PRESERVATIVE | 0.10 | 0.25 | 0.50 | 1.00 | 0 | 0 | 0.10 | 0.05 | 0 |
| (Table 2) | | | | | | | | | |
| Water | ad | ad | ad | ad | ad | ad | ad | ad | ad |
| | 100 | 100 | 100 | 100 | 100 | 100 | 100 | 100 | 100 |

Soap-containing deodorant stick

| | A | B | C | D | E | F | G | H |
|---|---|---|---|---|---|---|---|---|
| | % | % | % | % | % | % | % | % |
| Ethanol | 22.5 | 22.5 | 22.5 | 22.5 | 22.5 | 22.5 | 22.5 | 22.5 |
| Cutina ® | 4.4 | 4.4 | 4.4 | 4.4 | 4.4 | 4.4 | 4.4 | 4.4 |
| FS 45 | | | | | | | | |
| 1,3-Butanediol | 31.7 | 31.7 | 31.7 | 31.7 | 31.7 | 31.7 | 31.7 | 31.7 |
| 1,2-Propylene | 21.0 | 21.0 | 21.0 | 21.0 | 21.0 | 21.0 | 21.0 | 21.0 |
| glycol | | | | | | | | |
| Eutanol ® G | 2.0 | 2.0 | 2.0 | 2.0 | 2.0 | 2.0 | 2.0 | 2.0 |
| Aethoxal ® B | 3.0 | 3.0 | 3.0 | 3.0 | 3.0 | 3.0 | 3.0 | 3.0 |
| Cremophor ® | 0.05 | 0.05 | 0.05 | 0.05 | 0.05 | 0.05 | 0.05 | 0.05 |
| RH 455 | | | | | | | | |
| NaOH 45% | 1.44 | 1.44 | 1.44 | 1.44 | 1.44 | 1.44 | 1.44 | 1.44 |
| Phenoxy- | 1.0 | 1.0 | 1.0 | 1.0 | 1.0 | 1.0 | 1.0 | 1.0 |
| ethanol | | | | | | | | |
| Sensiva ® | 0.3 | 0.3 | 0.3 | 0.3 | 0.3 | 0.3 | 0.3 | 0.3 |
| SC 50 | | | | | | | | |
| Perfume oil | 2.0 | 2.0 | 2.0 | 2.0 | 2.0 | 2.0 | 2.0 | 2.0 |
| Pyrano- | 0.05 | 0.10 | 0.20 | 0.25 | 0.50 | 1.00 | 0.10 | 0.30 |
| pyranone | | | | | | | | |
| derivative | | | | | | | | |
| (Table 1) | | | | | | | | |
| BIOCIDE/ | 0.50 | 0.30 | 0.10 | 0.05 | 0.50 | 0 | 0 | 0.25 |
| PRESERVA- | | | | | | | | |
| TIVE | | | | | | | | |
| (Table2) | | | | | | | | |
| Water dist. | ad | ad | ad | ad | ad | ad | ad | ad |
| | 100 | 100 | 100 | 100 | 100 | 100 | 100 | 100 |

Deodorant Pump Atomizer

| | A | B | C | D | E | F | G |
|---|---|---|---|---|---|---|---|
| | % | % | % | % | % | % | % |
| Ethanol 96%, | 55.0 | 55.0 | 55.0 | 55.0 | 55.0 | 55.0 | 55.0 |
| (DEP denatured) | | | | | | | |
| Triethyl citrate | 3.0 | 3.0 | 3.0 | 3.0 | 3.0 | 3.0 | 3.0 |
| Cremophor ® RH 455 | 0.5 | 0.5 | 0.5 | 0.5 | 0.5 | 0.5 | 0.5 |
| BIOCIDE/ | 0.05 | 0.10 | 0.20 | 0.25 | 0.50 | 0 | 0.80 |
| PRESERVATIVE | | | | | | | |
| (Table 2) | | | | | | | |
| Pyranopyranone | 1.00 | 0.05 | 0.10 | 0.25 | 0.50 | 0.25 | 0.50 |
| derivative | | | | | | | |
| (Table 1) | | | | | | | |
| Perfume oil | 1.0 | 1.0 | 1.0 | 1.0 | 1.0 | 1.0 | 1.0 |
| Water | ad | ad | ad | ad | ad | ad | ad |
| | 100 | 100 | 100 | 100 | 100 | 100 | 100 |

| | Non-aqueous doedorant spray | | |
|---|---|---|---|
| | 5.1 | 5.2 | 5.3 |
| | % | % | % |
| 2-Octyl dodecanol | 0.5 | 0.5 | 0.5 |
| Ethanol 99% (DEP denatured) | 38.1 | 39.495 | 38.85 |
| Pyranopyranone derivative (Table 1) | 0.05 | 0.10 | 0.30 |
| BIOCIDE/PRESERVATIVE (Table 2) | 0.50 | 0.10 | 0 |
| n-Butane | 60 | 60 | 60 |

Antiperspirant Roll-on

| | A | B | C | D | E | F |
|---|---|---|---|---|---|---|
| | % | % | % | % | % | % |
| Ethanol 96% (DEP denatured) | 30.0 | 30.0 | 30.0 | 30.0 | 30.0 | 30.0 |
| Mergital ® CS 11 | 2.0 | 2.0 | 2.0 | 2.0 | 2.0 | 2.0 |
| Eumulgin ® B 3 | 2.0 | 2.0 | 2.0 | 2.0 | 2.0 | 2.0 |
| Aluminium chlorohydrate | 20.0 | 20.0 | 20.0 | 20.0 | 20.0 | 20.0 |
| Hydroxyethylcellulose | 0.5 | 0.5 | 0.5 | 0.5 | 0.5 | 0.5 |
| Pyranopyranone derivative | 0.05 | 0.10 | 0.25 | 0.50 | 0.10 | 0.50 |
| (Table 1) | | | | | | |
| BIOCIDE/PRESERVATIVE | 1.00 | 0.50 | 0.25 | 0.10 | 0 | 0 |
| (Table 2) | | | | | | |
| Perfume oil | 0.8 | 0.8 | 0.8 | 0.8 | 0.8 | 0.8 |
| Water | ad | ad | ad | ad | ad | ad |
| | 100 | 100 | 100 | 100 | 100 | 100 |

Antiperspirant spray suspension

| | A | B | C |
|---|---|---|---|
| | % | % | % |
| DC-245 | 10.0 | 10.0 | 10.0 |
| Isopropyl myristate | 5.0 | 5.0 | 5.0 |
| Aluminium chlorohydrate powder | 5.0 | 5.0 | 5.0 |
| Aerosil ® R 972 | 2.0 | 2.0 | 2.0 |
| Pyranopyranone derivative (Table 1) | 0.05 | 0.10 | 0.25 |
| BIOCIDE/PRESERVATIVE (Table 2) | 0.50 | 1.00 | 0 |
| n-Butane | 77.9 | 77.995 | 77.9 |

Transparent Antiperspirant Gel

| | | A | B | C | D | E |
|---|---|---|---|---|---|---|
| | | % | % | % | % | % |
| Phase 1 | DC-245 | 7.0 | | | | |
| | DC-3225 | 10.0 | | | | |
| | Pyranopyranone derivative | 0.05 | 0.10 | 0.20 | 0.50 | 0.25 |
| | (Table 1) | | | | | |
| | BIOCIDE/ | 1.00 | 0.50 | 0.10 | 0 | 0 |
| | PRESERVATIVE | | | | | |
| | (Table 2) | | | | | |
| Phase 2 | Chlorhydrol ® | 50.0 | | | | |
| | 1,2-Propylene glycol | 16.0 | | | | |
| | Water | 16.9 | | | | |

List of raw materials used

| | | |
|---|---|---|
| Aerosil R 972 | Silica dimethyl silylate | Degussa |
| Aethoxal ® B | PPG-5 laureth-5 | Cognis |
| Cetiol ® OE | Dicaprylyl ether | Cognis |
| Chlorhydrol ® | 50% Aluminium | Reheis |
| | chlorohydrate, 50% water | |
| Cremophor ® RH 455 | Hydrogenated castor oil with | BASF |
| | 40 EO, containing propylene | |
| | glycol | |
| Cutina ® FS 45 | Fatty acid mixture of palmitic | Cognis |
| | and stearic acid | |
| Cutina ® E 24 PF | PEG-20 glyceryl stearate | Cognis |
| Cutina ® HR | Hydrogenated castor oil | Cognis |
| DC ® 245 | Cyclopentasiloxane | Dow Corning |
| DC ® 3225 | Cyclomethicone/dimethicone | Dow Corning |
| | copolyol | |
| Eumulgin ® B 3 | Ceteareth-30 | Cognis |
| Eutanol ® G | 2-Octyl dodecanol | Cognis |
| Eutanol ® G 16 | 2-Hexyl decanol | Cognis |
| Lanette ® O | Cetyl/stearyl alcohol in a ratio | Cognis |
| | of 1:1 | |
| Lorol ® C 18 | Stearyl alcohol | Cognis |
| Mergital ® CS 11 | Ceteareth-11 | Cognis |
| Plantaren ® 1200 | Lauryl glucoside, about 50% | Cognis |
| | active substance | |
| Plantaren ® 2000 | Decyl glucoside, about 50% | Cognis |
| | active subst. | |
| Sensiva ® SC 50 | 2-Ethylhexylglycerol ether | Schülke & Mayr |
| Ucon Fluid ® AP | PPG-14 butyl ether | Amerchol (Union |
| | | Carbide) |

Anti-bacterial Fabric Softener

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.10 | 0.25 | 0.50 | 0.10 | 0.25 | 0.50 | 0.10 | 0.25 | 0.50 |
| (Table 1) | | | | | | | | | |
| C12-15 Pareth-7 | 0.50 | 0.50 | 0.50 | 0.50 | 0.50 | 0.50 | 0.50 | 0.50 | 0.50 |
| Quaternium 18 (and) Iso- | 4.00 | 4.00 | 4.00 | 4.00 | 4.00 | 4.00 | 4.00 | 4.00 | 4.00 |
| propyl Alcohol | | | | | | | | | |
| Water | qs to 100 | qs to 100 | qs to 100 | qs to 100 | qs to 100 | qs to 100 | qs to 100 | qs to 100 | qs to 100 |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| Perfume | qs | qs | qs | qs | qs | qs | qs | qs | qs |

Concentrated Fabric Softener

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.50 | 0.50 | 0.50 | 0.50 | 0.50 | 0.50 | 0.50 | 0.50 | 0.50 |
| (Table 1) | | | | | | | | | |
| Dihydrogenated Tallowethyl | 15.00 | 15.00 | 15.00 | 15.00 | 15.00 | 15.00 | 15.00 | 15.00 | 15.00 |
| Hydroxyethylamonium | | | | | | | | | |
| Oleth-10 | 2.00 | 2.00 | 2.00 | 2.00 | 2.00 | 2.00 | 2.00 | 2.00 | 2.00 |
| Simethicone | 0.50 | 0.50 | 0.50 | 0.50 | 0.50 | 0.50 | 0.50 | 0.50 | 0.50 |
| Isopropyl Alcohol | 3.00 | 3.00 | 3.00 | 3.00 | 3.00 | 3.00 | 3.00 | 3.00 | 3.00 |
| Magnesium Chloride | 1.00 | 1.00 | 1.00 | 1.00 | 1.00 | 1.00 | 1.00 | 1.00 | 1.00 |
| Water | qs to 100 | qs to 100 | qs to 100 | qs to 100 | qs to 100 | qs to 100 | qs to 100 | qs to 100 | qs to 100 |
| Perfume | qs | qs | qs | qs | qs | qs | qs | qs | qs |
| BIOCIDE/PRESERVATIVE | 0.00 | 0.10 | 0.20 | 0.30 | 0.40 | 0.50 | 0.60 | 0.70 | 0.80 |
| (Table 2) | | | | | | | | | |

Softener

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Stepantex VL 90A (Esterquat) | 17.20 | 17.20 | 17.20 | 17.20 | 17.20 | 17.20 | 17.20 | 17.20 | 17.20 |
| TINOTEX ® CMA | 1.00 | 1.00 | 1.00 | 1.00 | 1.00 | 1.00 | 1.00 | 1.00 | 1.00 |
| (Silicone emulsion) | | | | | | | | | |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |
| IRAGON ® Blue ABL80 sol. | 0.30 | 0.30 | 0.30 | 0.30 | 0.30 | 0.30 | 0.30 | 0.30 | 0.30 |
| 1% (Acid blue 80) | | | | | | | | | |
| Perfume | 0.40 | 0.40 | 0.40 | 0.40 | 0.40 | 0.40 | 0.40 | 0.40 | 0.40 |
| Calcium chloride 25% | 0.15 | 0.15 | 0.15 | 0.15 | 0.15 | 0.15 | 0.15 | 0.15 | 0.15 |
| solution | | | | | | | | | |
| Tap water | qs to 100 | qs to 100 | qs to 100 | qs to 100 | qs to 100 | qs to 100 | qs to 100 | qs to 100 | qs to 100 |

Liquid Laundry Detergent

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.10 | 0.20 | 0.30 | 0.40 | 0.50 | 0.60 | 0.70 | 0.80 | 1.00 |
| (Table 1) | | | | | | | | | |
| Sodium Dodecylbenzene- | 8.7 | 8.7 | 8.7 | 8.7 | 8.7 | 8.7 | 8.7 | 8.7 | 8.7 |
| sulfonate | | | | | | | | | |
| Trideceth-12 | 3.50 | 3.50 | 3.50 | 3.50 | 3.50 | 3.50 | 3.50 | 3.50 | 3.50 |
| Penta Sodium Triphosphate | 24.00 | 24.00 | 24.00 | 24.00 | 24.00 | 24.00 | 24.00 | 24.00 | 24.00 |
| Sodium Sulfate | 1.20 | 1.20 | 1.20 | 1.20 | 1.20 | 1.20 | 1.20 | 1.20 | 1.20 |
| Water (and) Sodium | 1.60 | 1.60 | 1.60 | 1.60 | 1.60 | 1.60 | 1.60 | 1.60 | 1.60 |
| Hydroxide | | | | | | | | | |
| Glycerin | 1.00 | 1.00 | 1.00 | 1.00 | 1.00 | 1.00 | 1.00 | 1.00 | 1.00 |
| Oleic Acid | 0.90 | 0.90 | 0.90 | 0.90 | 0.90 | 0.90 | 0.90 | 0.90 | 0.90 |
| Sodium Borate | 1.80 | 1.80 | 1.80 | 1.80 | 1.80 | 1.80 | 1.80 | 1.80 | 1.80 |
| Cellulose Gum | 0.40 | 0.40 | 0.40 | 0.40 | 0.40 | 0.40 | 0.40 | 0.40 | 0.40 |
| Water | qs to 100 | qs to 100 | qs to 100 | qs to 100 | qs to 100 | qs to 100 | qs to 100 | qs to 100 | qs to 100 |
| BIOCIDE/PRESERVATIVE | 0.00 | 0.00 | 0.00 | 0.25 | 0.25 | 0.25 | 0.50 | 0.50 | 0.50 |
| (Table 2) | | | | | | | | | |

Heavy Duty Liquid Detergent

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Pyranopyranone derivative | 0.10 | 0.20 | 0.30 | 0.40 | 0.50 | 0.60 | 0.70 | 0.80 | 1.00 |
| (Table 1) | | | | | | | | | |
| Sodium | 15.00 | 15.00 | 15.00 | 15.00 | 15.00 | 15.00 | 15.00 | 15.00 | 15.00 |
| Dodecylbenzenesulfonate | | | | | | | | | |
| C14-15 Pareth-7 | 14.00 | 14.00 | 14.00 | 14.00 | 14.00 | 14.00 | 14.00 | 14.00 | 14.00 |
| SD Alcohol 39-C | 9.00 | 9.00 | 9.00 | 9.00 | 9.00 | 9.00 | 9.00 | 9.00 | 9.00 |
| Sodium Tallowate (and) | 10.00 | 10.00 | 10.00 | 10.00 | 10.00 | 10.00 | 10.00 | 10.00 | 10.00 |
| Sodium Cocoate | | | | | | | | | |
| Trisodium Citrate Dihydrate | 4.00 | 4.00 | 4.00 | 4.00 | 4.00 | 4.00 | 4.00 | 4.00 | 4.00 |
| Triethanolamine | 5.00 | 5.00 | 5.00 | 5.00 | 5.00 | 5.00 | 5.00 | 5.00 | 5.00 |
| Fluorescent Whitening | 0.10 | 0.10 | 0.10 | 0.10 | 0.10 | 0.10 | 0.10 | 0.10 | 0.10 |
| Agent (FWA) | | | | | | | | | |
| Water | qs to 100 | qs to 100 | qs to 100 | qs to 100 | qs to 100 | qs to 100 | qs to 100 | qs to 100 | qs to 100 |
| BIOCIDE/PRESERVATIVE | 0.50 | 0.20 | 0.00 | 0.25 | 0.25 | 0.25 | 0.50 | 0 | 0 |
| (Table 2) | | | | | | | | | |

Liquid Laundry Detergent

| | | A | B | C | D | E | F | G | H | I |
|---|---|---|---|---|---|---|---|---|---|---|
| | | % | % | % | % | % | % | % | % | % |
| | | w/w | w/w | w/w | w/w | w/w | w/w | w/w | w/w | w/w |
| | Lauryl-myristyl sulfate | 25.00 | — | 25.00 | — | 25.00 | — | 25.00 | — | 25.00 |
| | Na-salt (Texapon LS35) | | | | | | | | | |
| | Alkyl Ether Sulfate Sodium | — | 8.00 | — | 5.00 | — | 10.00 | — | 15.00 | — |
| | salt | | | | | | | | | |
| | Coconut Fatty Acid | 5.00 | 5.00 | 5.00 | 5.00 | 5.00 | 5.00 | 5.00 | 5.00 | 5.00 |
| | (Edenor K12-18) | | | | | | | | | |
| | C-14,15 alcohol ethoxylate | 20.00 | 20.00 | 20.00 | 20.00 | 20.00 | 20.00 | 20.00 | 20.00 | 20.00 |
| | 7 EO (Neodol 45-7) | | | | | | | | | |
| Part B | Isopropyl Alcohol | 6.00 | 6.00 | 6.00 | 6.00 | 6.00 | 6.00 | 6.00 | 6.00 | 6.00 |
| | Pyranopyranone derivative | 0.10 | 0.25 | 0.50 | 0.10 | 0.25 | 0.50 | 0.10 | 0.25 | 0.50 |
| | (Table 1) | | | | | | | | | |
| | BIOCIDE/ | 0.25 | 0.00 | 0.05 | 0.25 | 0.25 | 0.25 | 0.50 | 0.00 | 0.00 |
| | PRESERVATIVE (Table 2) | | | | | | | | | |
| | Propylene Glycol | 2.00 | 2.00 | 2.00 | 2.00 | 2.00 | 2.00 | 2.00 | 2.00 | 2.00 |
| | Ethanolamine | 3.00 | 3.00 | 3.00 | 3.00 | 3.00 | 3.00 | 3.00 | 3.00 | 3.00 |
| | Triethanolamine | 2.00 | 2.00 | 2.00 | 2.00 | 2.00 | 2.00 | 2.00 | 2.00 | 2.00 |
| | Citric acid (sol. 50%) | 6.00 | 6.00 | 6.00 | 6.00 | 6.00 | 6.00 | 6.00 | 6.00 | 6.00 |
| | Potassium Hydroxide | 1.00 | 1.00 | 1.00 | 1.00 | 1.00 | 1.00 | 1.00 | 1.00 | 1.00 |
| | (sol. 45%) | | | | | | | | | |
| | Fluorescent Whitening | 0.15 | 0.15 | 0.15 | 0.15 | 0.15 | 0.15 | 0.15 | 0.15 | 0.15 |
| | Agent (FWA) | | | | | | | | | |
| | Acid blue | 0.20 | 0.20 | 0.20 | 0.20 | 0.20 | 0.20 | 0.20 | 0.20 | 0.20 |
| | Anionic acrylic polymer | 3.00 | 3.00 | 3.00 | 3.00 | 3.00 | 3.00 | 3.00 | 3.00 | 3.00 |
| | (Rheovis ATA) | | | | | | | | | |
| | Deionized Water | Qs | Qs | Qs | Qs to 100 | Qs to 100 | Qs to 100 | Qs to 100 | Qs to 100 | Qs to 100 |
| | | to 100 | to 100 | to 100 | | | | | | |

### Manufacturing Instruction

Mix 2-Propanol and Propylene glycol. Then add successively while stirring Edenor (melted at 40° C.), Texapon, Triethanolamine, Ethanolamine, Neodol, potassium hydroxide and citric acid.

Finally add deionized water to the former solution while powerful stirring. Incorporate TINOPAL, IRAGON Blue, followed by RHEOVIS ATA slowly while stirring

Powdery Laundry Detergent

| | | A | B | C | D | E | F | G | H | I |
|---|---|---|---|---|---|---|---|---|---|---|
| | | % | % | % | % | % | % | % | % | % |
| | | w/w | w/w | w/w | w/w | w/w | w/w | w/w | w/w | w/w |
| | Alkyl benzene | 7.00 | 7.00 | 7.00 | 7.00 | 7.00 | 7.00 | 7.00 | 7.00 | 7.00 |
| | sulfonate, sodium salt | | | | | | | | | |
| | (Marlon A 375) | | | | | | | | | |
| | Alkyl Ether Sulfate Sodium | 4.00 | 4.00 | 4.00 | 4.00 | 4.00 | 4.00 | 4.00 | 4.00 | 4.00 |
| | salt (Empicol ESC | | | | | | | | | |
| | 70) | | | | | | | | | |
| | Coconut Fatty Acid | 2.00 | 2.00 | 2.00 | 2.00 | 2.00 | 2.00 | 2.00 | 2.00 | 2.00 |
| | (Edenor K12-18) | | | | | | | | | |
| | C14-15 Pareth-7 | 8.00 | 8.00 | 8.00 | 8.00 | 8.00 | 8.00 | 8.00 | 8.00 | 8.00 |
| | (Neodol 45-7 E) | | | | | | | | | |
| Part B | Sodium Aluminium Silicate | 30.00 | 30.00 | 30.00 | 30.00 | 30.00 | 30.00 | 30.00 | 30.00 | 30.00 |
| | (zeolithe 4 A) | | | | | | | | | |
| | Sodium Carbonate | 20.00 | 20.00 | 20.00 | 20.00 | 20.00 | 20.00 | 20.00 | 20.00 | 20.00 |
| | Sodium Sulfate | 10.00 | 10.00 | 10.00 | 10.00 | 10.00 | 10.00 | 10.00 | 10.00 | 10.00 |
| | Acrylic acid based | 4.00 | 4.00 | 4.00 | 4.00 | 4.00 | 4.00 | 4.00 | 4.00 | 4.00 |
| | polymer (Antiprex | | | | | | | | | |
| | A100) | | | | | | | | | |
| | Sodium Silicate | 12.00 | 12.00 | 12.00 | 12.00 | 12.00 | 12.00 | 12.00 | 12.00 | 12.00 |
| | (Britesil C20) | | | | | | | | | |
| | Pyranopyranone derivative | 0.10 | 0.25 | 0.50 | 0.10 | 0.25 | 0.50 | 0.10 | 0.25 | 0.50 |
| | (Table 1) | | | | | | | | | |
| | BIO- | 0.20 | 0.10 | 0.00 | 0.00 | 0.00 | 0.00 | 0.00 | 0.00 | 0.00 |
| | CIDE/PRESERVATIVE | | | | | | | | | |
| | (Table 2) | | | | | | | | | |
| | TINOFIX CL SOLID | 1.00 | 1.00 | 1.00 | 1.00 | 1.00 | 1.00 | 1.00 | 1.00 | 1.00 |
| | Deionized Water | Qs | Qs | Qs | Qs | Qs to 100 | Qs to 100 | Qs to 100 | Qs to 100 | Qs to 100 |
| | | to 100 | to 100 | to 100 | to 100 | | | | | |

Manufacturing Instruction:

Make two premixes:

A 30% solution of Marlon in deionized water is mixed with 50% of the amount of Zeolith, dried in oven and crushed to small pieces.

A 30% solution of Empicol in deionized water is mixed with 50% of the amount of Zeolith, dried in oven and crushed to small pieces.

The dried premixes are mixed with Antiprex, Britesil and Sodium carbonate. The melted Neodol and Edenor are added step by step, then Sodium sulfate and finally TINOFIX CL solid.

Laundry Sanitizer

| | | A | B | C | D | E | F | G | H | I |
|---|---|---|---|---|---|---|---|---|---|---|
| | | % | % | % | % | % | % | % | % | % |
| | | w/w | w/w | w/w | w/w | w/w | w/w | w/w | w/w | w/w |
| | Sodium Lauryl Sulfate | 2.00 | 2.00 | 2.00 | 2.00 | 2.00 | 2.00 | 2.00 | 2.00 | 2.00 |
| | Sodium Cumene Sulfonate | 5.00 | 5.00 | 5.00 | 5.00 | 5.00 | 5.00 | 5.00 | 5.00 | 5.00 |
| | Water deioniz | Qs | Qs | Qs | Qs to 100 | Qs to 100 | Qs to 100 | Qs to 100 | Qs to 100 | Qs to 100 |
| | | to 100 | to 100 | to 100 | | | | | | |
| Part B | PPG-2 Methyl Ether | 10.00 | 10.00 | 10.00 | 10.00 | 10.00 | 10.00 | 10.00 | 10.00 | 10.00 |
| | (Dowanol DPM) | | | | | | | | | |
| | Propylene Glycol | 10.00 | 10.00 | 10.00 | 10.00 | 10.00 | 10.00 | 10.00 | 10.00 | 10.00 |
| | Color and Perfume | qs | qs | qs | qs | qs | qs | qs | qs | qs |
| § | Citric Acid | 3.00 | 3.00 | 3.00 | 3.00 | 3.00 | 3.00 | 3.00 | 3.00 | 3.00 |
| | Pyranopyranone derivative | 0.10 | 0.25 | 0.50 | 0.10 | 0.25 | 0.50 | 0.10 | 0.25 | 0.50 |
| | (Table 1) | | | | | | | | | |
| | BIO- | 0.00 | 0.00 | 0.00 | 0.25 | 0.25 | 0.25 | 0.50 | 0.00 | 0.00 |
| | CIDE/PRESERVATIVE | | | | | | | | | |
| | (Table 2) | | | | | | | | | |
| | Sodium Hydroxide | Qs | Qs | Qs | Qs | Qs | Qs | Qs | Qs | Qs |
| | (10% solution) | to | to | to | to | to | to | to | to | to |
| | | pH 5 | pH 5 | pH 5 | pH 5 | pH 5 | pH 5 | pH 5 | pH 5 | pH 5 |

Antibacterial Liquid Cleaner

| | A | B | C | D | E | |
|---|---|---|---|---|---|---|
| | % | % | % | % | % | F |
| | w/w | w/w | w/w | w/w | w/w | % w/w |
| Octylalcohol 4EO | 3.00 | 5.00 | 1.00 | 3.00 | 5.00 | 1.00 |
| Fatty alcohol C₈-C₁₀- | 1.00 | 1.50 | 3.00 | 1.50 | 1.00 | 5.00 |
| Polyglucosid | | | | | | |
| Isopropanol | 3.00 | 3.00 | 10.00 | 1.00 | 1.00 | 5.00 |
| Pyranopyranone | 0.10 | 0.10 | 0.10 | 0.50 | 0.50 | 0.50 |
| derivative (Table 1) | | | | | | |
| BIOCIDE/ | 0.20 | 0.00 | 0.00 | 0.00 | 0.00 | 0.00 |
| PRESERVATIVE (Table 2) | | | | | | |
| Water | 92.90 | 90.40 | 85.90 | 94.00 | 92.50 | 88.50 |

Antibacterial Dishwashing Paste

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Water | 24.00 | 24.00 | 24.00 | 24.00 | 24.00 | 24.00 | 24.00 | 24.00 | 24.00 |
| Sodium Carbonate | 16.00 | 16.00 | 16.00 | 16.00 | 16.00 | 16.00 | 16.00 | 16.00 | 16.00 |
| Calcium Carbonate | 28.00 | 28.00 | 28.00 | 28.00 | 28.00 | 28.00 | 28.00 | 28.00 | 28.00 |
| Sodium Silicate | 5.00 | 5.00 | 5.00 | 5.00 | 5.00 | 5.00 | 5.00 | 5.00 | 5.00 |
| Nonoxynol-10 | 0.50 | 0.50 | 0.50 | 0.50 | 0.50 | 0.50 | 0.50 | 0.50 | 0.50 |
| Fragrance | 0.20 | 0.20 | 0.20 | 0.20 | 0.20 | 0.20 | 0.20 | 0.20 | 0.20 |
| Pyranopyranone derivative | 0.10 | 0.20 | 0.30 | 0.40 | 0.50 | 0.60 | 0.70 | 0.80 | 1.00 |
| (Table 1) | | | | | | | | | |
| BIO- | 0.00 | 0.00 | 0.00 | 0.25 | 0.25 | 0.25 | 0.00 | 0.00 | 0.00 |
| CIDE/PRESERVATIVE | | | | | | | | | |
| (Table 2) | | | | | | | | | |
| C.I.15510 (Acid Orange 7) | 0.10 | 0.10 | 0.10 | 0.10 | 0.10 | 0.10 | 0.10 | 0.10 | 0.10 |
| Glycerin 85% | 1.00 | 1.00 | 1.00 | 1.00 | 1.00 | 1.00 | 1.00 | 1.00 | 1.00 |
| Dodecylbenzene Sulfonic | 25.00 | 25.00 | 25.00 | 25.00 | 25.00 | 25.00 | 25.00 | 25.00 | 25.00 |
| Acid | | | | | | | | | |

Bactericidal Dishwashing Liquid

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.10 | 0.25 | 0.50 | 0.10 | 0.25 | 0.50 | 0.10 | 0.25 | 0.50 |
| (Table 1) | | | | | | | | | |
| BIO- | 0.00 | 0.00 | 0.00 | 0.25 | 0.25 | 0.25 | 0.50 | 0.50 | 0.50 |
| CIDE/PRESERVATIVE | | | | | | | | | |
| (Table 2) | | | | | | | | | |
| Sodium Laureth Sulfate | 50.00 | 50.00 | 50.00 | 50.00 | 50.00 | 50.00 | 50.00 | 50.00 | 50.00 |
| Sodium Dodecylbenzene- | 7.00 | 7.00 | 7.00 | 7.00 | 7.00 | 7.00 | 7.00 | 7.00 | 7.00 |
| sulfonate | | | | | | | | | |
| Sodium Cumene Sulfonate | 5.00 | 5.00 | 5.00 | 5.00 | 5.00 | 5.00 | 5.00 | 5.00 | 5.00 |
| Citric Acid | 3.00 | 3.00 | 3.00 | 3.00 | 3.00 | 3.00 | 3.00 | 3.00 | 3.00 |
| Benzoic Acid | 0.50 | 0.50 | 0.50 | 0.50 | 0.50 | 0.50 | 0.50 | 0.50 | 0.50 |
| Sodium Chloride | 1.00 | 1.00 | 1.00 | 1.00 | 1.00 | 1.00 | 1.00 | 1.00 | 1.00 |
| Sodium Sulfate | 3.50 | 3.50 | 3.50 | 3.50 | 3.50 | 3.50 | 3.50 | 3.50 | 3.50 |
| Water (and) Sodium | ad | ad | ad | ad | ad | ad | ad | ad | ad |
| Hydroxide | pH 5.0 | pH 5.0 | pH 5.0 | pH 5.0 | pH 5.0 | pH 5.0 | pH 5.0 | pH 5.0 | pH 5.0 |
| Water | qs to 100 | qs to 100 | qs to 100 | qs to 100 | qs to 100 | qs to 100 | qs to 100 | qs to 100 | qs to 100 |

Dishwashing liquid and Anti-bacterial Hand Soap

| | A | B | C | D | E | F | G | H | |
|---|---|---|---|---|---|---|---|---|---|
| | % | % | % | % | % | % | % | % | I |
| | w/w | w/w | w/w | w/w | w/w | w/w | w/w | w/w | % w/w |
| Pyranopyranone derivative | 0.10 | 0.25 | 0.50 | 0.10 | 0.25 | 0.50 | 0.10 | 0.25 | 0.50 |
| (Table 1) | | | | | | | | | |
| Lauryl-myristyl sulfate Na- | 7.00 | 7.00 | 7.00 | 7.00 | 7.00 | 7.00 | 7.00 | 7.00 | 7.00 |
| salt | | | | | | | | | |
| Sodium Myreth Sulfate | 7.00 | 7.00 | 7.00 | 7.00 | 7.00 | 7.00 | 7.00 | 7.00 | 7.00 |
| Lauryl Glucoside | 4.00 | 4.00 | 4.00 | 4.00 | 4.00 | 4.00 | 4.00 | 4.00 | 4.00 |
| Coco Betaine | 1.1 | 1.1 | 1.1 | 1.1 | 1.1 | 1.1 | 1.1 | 1.1 | 1.1 |
| SD Alcohol 39-C | 5.00 | 5.00 | 5.00 | 5.00 | 5.00 | 5.00 | 5.00 | 5.00 | 5.00 |
| Sodium Chloride | 1.00 | 1.00 | 1.00 | 1.00 | 1.00 | 1.00 | 1.00 | 1.00 | 1.00 |
| Citric Acid | ad | ad | ad | ad | ad | ad | ad | ad | ad |
| | pH 6.5 | pH 6.5 | pH 6.5 | pH 6.5 | pH 6.5 | pH 6.5 | pH 6.5 | pH 6.5 | pH 6.5 |
| BIO- | 0.10 | 0.10 | 0.10 | 0.25 | 0.25 | 0.25 | 0.00 | 0.00 | 0.00 |
| CIDE/PRESERVATIVE | | | | | | | | | |
| (Table 2) | | | | | | | | | |
| Water | qs to 100 | qs to 100 | qs to 100 | qs to 100 | qs to 100 | qs to 100 | qs to 100 | qs to 100 | qs to 100 |

Transparent Acidic Cleaner

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Capryleth-4 (Dehydol 04 Deo) | 2.00 | 2.00 | 2.00 | 2.00 | 2.00 | 2.00 | 2.00 | 2.00 | 2.00 |
| Alkyl Polyglycoside | 8.00 | 8.00 | 8.00 | 8.00 | 8.00 | 8.00 | 8.00 | 8.00 | 8.00 |
| (Glucopon 215 CS UP) | | | | | | | | | |
| Acetic acid sol. 50% | 5.0 | 5.0 | 5.0 | 5.0 | 5.0 | 5.0 | 5.0 | 5.0 | 5.0 |
| Citric acid sol. 50% | 5.00 | 5.00 | 5.00 | 5.00 | 5.00 | 5.00 | 5.00 | 5.00 | 5.00 |
| Acid Green 25 VI- | 0.20 | 0.20 | 0.20 | 0.20 | 0.20 | 0.20 | 0.20 | 0.20 | 0.20 |
| BRACOLOR ® Green | | | | | | | | | |
| AGR25 (1% sol) | | | | | | | | | |
| Cationic Polymer | 2.00 | 2.00 | 2.00 | 2.00 | 2.00 | 2.00 | 2.00 | 2.00 | 2.00 |
| RHEOVIS ® CSP | | | | | | | | | |
| Pyranopyranone derivative | 0.05 | 0.1 | 0.2 | 0.3 | 0.4 | 0.5 | 0.75 | 0.85 | 1 |
| (Table 1) | | | | | | | | | |
| BIOCIDE/PRESERVATIVE | 0.50 | 0.25 | 0.10 | 0.00 | 0.00 | 0.10 | 0.00 | 0.00 | 0.00 |
| (Table 2) | | | | | | | | | |
| Deionized Water | Qs to 100 | Qs to 100 | Qs to 100 | Qs to 100 | Qs to 100 | Qs to 100 | Qs to 100 | Qs to 100 | Qs to 100 |

Milky acidic Cleaner

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| C-12,15 alcohol ethoxylate | 2.50 | 2.50 | 2.50 | 2.50 | 2.50 | 2.50 | 2.50 | 2.50 | 2.50 |
| 7 EO (Neodol 25-7E) | | | | | | | | | |
| C-12,13 alcohol ethoxylate | 2.5 | 2.5 | 2.5 | 2.5 | 2.5 | 2.5 | 2.5 | 2.5 | 2.5 |
| 6.5 EO (Neodol 23-6.5E) | | | | | | | | | |
| Citric acid sol. 50% | 5.00 | 5.00 | 5.00 | 5.00 | 5.00 | 5.00 | 5.00 | 5.00 | 5.00 |
| Acid Green 25 VI- | 0.20 | 0.20 | 0.20 | 0.20 | 0.20 | 0.20 | 0.20 | 0.20 | 0.20 |
| BRACOLOR ® Green | | | | | | | | | |
| AGR25 (1% sol) | | | | | | | | | |
| Cationic Polymer | 2.00 | 2.00 | 2.00 | 2.00 | 2.00 | 2.00 | 2.00 | 2.00 | 2.00 |
| RHEOVIS ® CDP | | | | | | | | | |
| Pyranopyranone derivative | 0.05 | 0.1 | 0.2 | 0.3 | 0.4 | 0.5 | 0.75 | 0.85 | 1 |
| (Table 1) | | | | | | | | | |
| BIOCIDE/PRESERVATIVE | 0.50 | 1.00 | 0.00 | 0.00 | 0.00 | 0.00 | 0.00 | 0.10 | 0.00 |
| (Table 2) | | | | | | | | | |
| Deionized Water | Qs | Qs to 100 | Qs to 100 | Qs to 100 | Qs to 100 | Qs to 100 | Qs to 100 | Qs to 100 | Qs to 100 |
| | to 100 | | | | | | | | |

Manufacturing Instructions

Firstly add Neodol 25-7 to the water at 40° C. while stirring (100 rpm) and wait until the formulation is homogeneous, then add Neodol 23-6.5 and wait until the formulation is homogeneous. Stop heating.

Finally add citric acid and after 5 minutes incorporate VIBRACOLOR Green and RHEOVIS CDP slowly while stirring.

Desinfectant Toilet Cleaner

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Alkyl Polyglycoside | 3.50 | 3.50 | 3.50 | 3.50 | 3.50 | 3.50 | 3.50 | 3.50 | 3.50 |
| C9-11 pareth-8 | 1.70 | 1.70 | 1.70 | 1.70 | 1.70 | 1.70 | 1.70 | 1.70 | 1.70 |
| (Neodol 91-8E) | | | | | | | | | |
| Cationic Polymer | 11.30 | 11.30 | 11.30 | 11.30 | 11.30 | 11.30 | 11.30 | 11.30 | 11.30 |
| Rheovis ® CSP | | | | | | | | | |
| Acetic Acid | 2.40 | 2.40 | 2.40 | 2.40 | 2.40 | 2.40 | 2.40 | 2.40 | 2.40 |
| Citric Acid | 9.00 | 9.00 | 9.00 | 9.00 | 9.00 | 9.00 | 9.00 | 9.00 | 9.00 |
| Pyranopyranone derivative | 0.05 | 0.1 | 0.2 | 0.3 | 0.4 | 0.5 | 0.75 | 0.85 | 1 |
| (Table 1) | | | | | | | | | |
| BIOCIDE/PRESERVATIVE | 0.25 | 1.00 | 0.00 | 0.00 | 0.00 | 0.00 | 0.00 | 0.00 | 0.05 |
| (Table 2) | | | | | | | | | |
| Deionized Water | Qs | Qs to 100 | Qs to 100 | Qs to 100 | Qs to 100 | Qs to 100 | Qs to 100 | Qs to 100 | Qs to 100 |
| | to 100 | | | | | | | | |
| Acid blue 80 | 0.50 | 0.50 | 0.50 | 0.50 | 0.50 | 0.50 | 0.50 | 0.50 | 0.50 |
| (IRAGON ® Blue | | | | | | | | | |
| ABL80 sol. 1%) | | | | | | | | | |

All Purpose Cleaner

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Hydoxydichlorodiphenyl Ether | 0.30 | 0.30 | 0.30 | 0.30 | 0.30 | 0.30 | 0.30 | 0.30 | 0.30 |
| Alkoxylated Alcohol | 10.0 | 10.0 | 10.0 | 10.0 | 10.0 | 10.0 | 10.0 | 10.0 | 10.0 |
| Sodium C14-17 Alkyl Sec | 14.0 | 14.0 | 14.0 | 14.0 | 14.0 | 14.0 | 14.0 | 14.0 | 14.0 |
| Sulfonate | | | | | | | | | |
| Lauryl-myristyl sulfate Na-salt | 5.00 | 5.00 | 5.00 | 5.00 | 5.00 | 5.00 | 5.00 | 5.00 | 5.00 |
| Diethylene Glycol Monobutyl | 5.00 | 5.00 | 5.00 | 5.00 | 5.00 | 5.00 | 5.00 | 5.00 | 5.00 |
| Ether | | | | | | | | | |
| Pyranopyranone | 0.05 | 0.1 | 0.2 | 0.3 | 0.4 | 0.5 | 0.75 | 0.85 | 1 |
| derivative (Table 1) | | | | | | | | | |
| BIOCIDE/PRESERVATIVE | 0.50 | 0.10 | 0.00 | 0.00 | 0.00 | 0.00 | 0.00 | 0.00 | 0.05 |
| (Table 2) | | | | | | | | | |
| Citric Acid | ad | ad | ad | ad | ad | ad | ad | ad | ad |
| | pH 6.5 | pH 6.5 | pH 6.5 | pH 6.5 | pH 6.5 | pH 6.5 | pH 6.5 | pH 6.5 | pH 6.5 |
| Acid Blue | 0.01 | 0.01 | 0.01 | 0.01 | 0.01 | 0.01 | 0.01 | 0.01 | 0.01 |
| Benzotriazole Derivative | 0.05 | 0.05 | 0.05 | 0.05 | 0.05 | 0.05 | 0.05 | 0.05 | 0.05 |
| Perfume | qs | qs | qs | qs | qs | qs | qs | qs | qs |
| Water | qs | qs to 100 | qs to 100 | qs to 100 | qs to 100 | qs to 100 | qs to 100 | qs to 100 | qs to 100 |
| | to 100 | | | | | | | | |

Antibacterial All Purpose Cleaner

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.30 | 0.30 | 1.00 | 0.30 | 0.25 | 0.30 | 0.30 | 2.00 | 0.05 |
| (Table 1) | | | | | | | | | |
| Lauryl-myristyl sulfate | 10.00 | 10.00 | 10.00 | 10.00 | 10.00 | 10.00 | 10.00 | 10.00 | 10.00 |
| Na-salt | | | | | | | | | |
| Alkyl Polyglycoside | 3.00 | 3.00 | 3.00 | 3.00 | 3.00 | 3.00 | 3.00 | 3.00 | 3.00 |
| Sodium Cumene Sulfonate | 5.00 | 5.00 | 5.00 | 5.00 | 5.00 | 5.00 | 5.00 | 5.00 | 5.00 |
| BIOCIDE/PRESERVATIVE | 0 | 0.10 | 0 | 1.00 | 0 | 2.00 | 0.05 | 0 | 0 |
| (Table 2) | | | | | | | | | |
| Citric Acid | ad | ad | ad | ad | ad | ad | ad | ad | ad |
| | pH 3.0 | pH 3.0 | pH 3.0 | pH 3.0 | pH 3.0 | pH 3.0 | pH 3.0 | pH 3.0 | pH 3.0 |
| Water | qs | qs to 100 | qs to 100 | qs to 100 | qs to 100 | qs to 100 | qs to 100 | qs to 100 | qs to 100 |
| | to 100 | | | | | | | | |

Surface disinfectant spray

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.10 | 0.30 | 1.00 | 2.00 | 0.10 | 0.30 | 1.00 | 2.00 | 0.50 |
| (Table 1) | | | | | | | | | |
| Isopropyl Alcohol | 10.00 | 10.00 | 10.00 | 10.00 | 10.00 | 10.00 | 10.00 | 10.00 | 10.00 |
| Sodium Lauryl Sulfate | 1.00 | 1.00 | 1.00 | 1.00 | 1.00 | 1.00 | 1.00 | 1.00 | 1.00 |
| Citric Acid | 3.00 | 3.00 | 3.00 | 3.00 | 3.00 | 3.00 | 3.00 | 3.00 | 3.00 |
| BIOCIDE/PRESERVATIVE | 2.00 | 1.00 | 0.50 | 0.10 | 0.00 | 0.00 | 0.00 | 0.00 | 0.05 |
| (Table 2) | | | | | | | | | |
| Water (and) Sodium | ad | ad | ad | ad | ad | ad | ad | ad | ad |
| Hydroxide | pH 5.0 | pH 5.0 | pH 5.0 | pH 5.0 | pH 5.0 | pH 5.0 | pH 5.0 | pH 5.0 | pH 5.0 |
| Water | qs to 100 | qs to 100 | qs to 100 | qs to 100 | qs to 100 | qs to 100 | qs to 100 | qs to 100 | qs to 100 |

Wet wipes

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Pyranopyranone derivative | 0.01 | 0.05 | 0.10 | 0.50 | 0.01 | 0.05 | 0.10 | 0.50 | 1.00 |
| (Table 1) | | | | | | | | | |
| PEG-5 Glyceryl Stearate | 1.00 | 1.00 | 1.00 | 1.00 | 1.00 | 1.00 | 1.00 | 1.00 | 1.00 |
| Steareth-20 | 1.00 | 1.00 | 1.00 | 1.00 | 1.00 | 1.00 | 1.00 | 1.00 | 1.00 |
| Steareth-21 | 1.00 | 1.00 | 1.00 | 1.00 | 1.00 | 1.00 | 1.00 | 1.00 | 1.00 |
| Mineral Oil | 1.00 | 1.00 | 1.00 | 1.00 | 1.00 | 1.00 | 1.00 | 1.00 | 1.00 |
| Isopropyl Myristate | 1.00 | 1.00 | 1.00 | 1.00 | 1.00 | 1.00 | 1.00 | 1.00 | 1.00 |
| Perfume | qs | qs | qs | qs | qs | qs | qs | qs | qs |
| Propylene Glycol | 1.00 | 1.00 | 1.00 | 1.00 | 1.00 | 1.00 | 1.00 | 1.00 | 1.00 |
| Glycerin | 1.50 | 1.50 | 1.50 | 1.50 | 1.50 | 1.50 | 1.50 | 1.50 | 1.50 |
| BIOCIDE/PRESERVATIVE | 2.00 | 1.00 | 0.50 | 0.20 | 0 | 0 | 0 | 0 | 0 |
| (Table 2) | | | | | | | | | |
| Water | qs to 100 | qs to 100 | qs to 100 | qs to 100 | qs to 100 | qs to 100 | qs to 100 | qs to 100 | qs to 100 |

### EXAMPLES

### Example 1

### Preparation of P-7

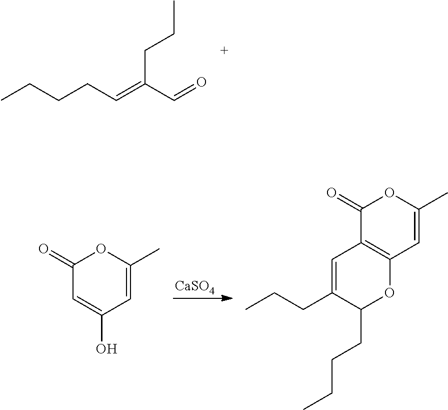

5.66 g (60.0 mmol) 4-hydroxy-6-methyl-2H-pyran-2-one, 6.36 g 2-propyl-2-heptenal, 1.80 g of -alanine and 2.75 g (90.0 mmol) calcium sulfate suspended in 200 ml of ethylacetate are heated under reflux for 24 hours. After cooling to room temperature the mixture is extracted with 100 mL of a saturated aqueous sodium hydrogen carbonate solution. The aqueous phase is extracted twice with 100 mL of ethyl acetate. The combined organic phases are washed with 200 mL brine and finally dried over sodium sulfate. The solvent is removed in vacuum. Purification was achieved by column chromatography (silica gel, toluene/ethyl acetate 9:1 as eluent). Yellow oil, yield 3.95 g.

UV-Vis (ethanol): ₘₐₓ=341 nm, =4039.

1H-NMR (CDCl₃, [ppm]): 6.19 (s, 1H), 5.80 (m, 1H), 4.79 (dd, 1H), 2.21 (d, 3H), 1.92-2.04 (m, 2H), 1.72-1.82 (m, 1H), 1.42-1.62 (m, 4H), 1.28-1.42 (m, 3H), 0.95 (t, 3H), 0.91 (t, 3H).

### Example 2

Tests for Antimicrobial Efficacy

The bactericidal activity of the compounds of the present invention is measured in a suspension test according to trivial modifications of the European Standard method EN 1040.

Substances listed in Table 1 are tested against gram positive and gram negative bacteria, yeasts and moulds. A selection of the results is shown in the following.

### Suspension Test

The pyrano pyranone derivatives are dissolved in ethanol or DMSO resulting in 1% stock solutions of the compounds.

The cell densities of the bacterial suspensions used as inoculums for the suspension test are as follows (cfu=colony forming units):

| | |
|---|---|
| Staphylococcus aureus ATCC 6538 | 1.8 × 10⁸ cfu/ml |
| Escherichia coli ATCC 10536 | 1.4 × 10⁸ cfu/ml |
| Pseudomonas aeruginosa ATCC15442 | 2.2 × 10⁸ cfu/ml |

For the suspension tests, an appropriate concentration of the test compound is mixed with a bacterial suspension resulting in a final cell count in the mixture of about 10⁵ cfu/ml. These test mixtures are incubated at room temperature (22° C.+/−2° C.) and aliquots of 1 ml test mixture containing the test bacteria were taken after set contact times for determination of the residual cell count of test bacteria. For inactivation of the antimicrobial compounds, the 1 ml aliquots are diluted in 1:10 steps in a solution with 10% Tween, 3% lecithin, 0.1% histidine and 0.5% sodium thiosulfate. The results of the determination of residual cell counts are given in the table below.

The fungicidal activity of the compounds of the present invention is measured in a suspension test according to trivial modifications of the European Standard method EN 1275.

The pyrano pyranone derivatives are dissolved in ethanol or DMSO resulting in 1% stock solutions of the compounds.

The cell density of Candida albicans ATCC 10231 used as inoculum for the suspension test was 2.4×10⁸ cfu/ml and of A. niger ATCC 6275 3.8×10⁸ cfu/ml.

For the suspension tests, an appropriate concentration of the test compound is mixed with a Candida albicans suspension resulting in a final cell count of about 10⁵ cfu/ml. These test mixtures are incubated at room temperature (22° C.+/−2° C.) and aliquots of 1 ml test mixture containing Candida albicans were taken after set contact times for determination of the residual cell count.

For inactivation of the antimicrobial compounds, the 1 ml aliquots are diluted in 1:10 steps in a solution with 10% Tween, 3% lecithin, 0.1% histidine and 0.5% sodium thiosulfate.

Aliquots of 1 ml were taken of the A. niger containing test mixtures after set contact times for determination of the residual cell count. For inactivation of the antimicrobial compounds, the 1 ml aliquots were diluted in 1:10 steps in a solution of the following composition:

| | | |
|---|---|---|
| 10% preformulation | preformulation: | 30% Tween 80 |
| 6.5% Sabouraudagar | | 3% lecithin |
| 0.1% Histidine | | |

The results of the determination of residual cell counts are given in the below table.

TABLE A
Results of the suspension tests
conducted to determine microbicidal activities

| | Test Compound (concentration tested) | | | | |
|---|---|---|---|---|---|
| | Com- | Com- | Com- | Com- | Com- |
| Test Organism*/ | pound | pound | pound | pound | pound |
| [Contact time] | P-6 | P-5 | P-4 | P-3 | P-1 |
| S. aureus/ | <100 | | <100 | <100 | <100 |
| [24 h] | >3 | | >3 | >3 | >3 |
| S. aureus/ | | <100 | | | |
| [7 d] | | >3 | | | |
| E. coli/ | <100 | 4.0 × 10² | <100 | <100 | <100 |
| [24 h] | >3 | >3 | >3 | >3 | >3 |
| P. aeruginosa/ | <100 | <100 | <100 | <100 | <100 |
| [24 h] | >3 | >3 | >3 | >3 | >3 |
| C. albicans/ | <100 | | <100 | <100 | <100 |
| [24 h] | >3 | | >3 | >3 | >3 |
| C. albicans/ | | <100 | | | |
| [7 d] | | >3 | | | |
| A. niger/ | <10 | | <10 | | 1.2 × 10³ |
| [24 h] | >3 | | >3 | | 2.2 |
| A. niger/ | | | | 1.2 × 10³ | |
| [7 d] | | | | 2.2 | |

*(cfu/ml and log reduction after set contact times)

The results show a strong microbicidal activity of the tested pyrano pyranone derivatives.

For determination of the microbistatic activity, the Minimum Inhibitory Concentrations (MIC) of the test compounds is determined.

The pyrano pyranone derivatives are also tested in an agar incorporation test in order to determine their minimum inhibitory concentrations (MIC) against various tests organisms. A selection of the results is shown in the following.

For this compound, a 1% stock solutions i9s prepared in ethanol.

A dilution series of the test compound in 15 ml still liquid agar media are prepared with 400 ppm, 200 ppm, 100 ppm, 50 ppm, 25 ppm, 12.5 ppm, 6.25 ppm, 3.125 ppm and 1.56 pmm of active.

The media used for the agar plates are Müller-Hinton (Merck) for the determination of the MIC against bacteria and C. albicans and Sabouraud Agar (Merck) for the determination of the MIC against A. niger. For the determination of the MIC against Malassezia furfur DSM 6171, CIP 1636-86 Medium 472 (DSMZ—Deutsche Sammlung von Mikroorganismen and Zellkulturen GmbH) was used.

After solidification of the agar, all agar plates containing the test compounds were stored over night in the laboratory (at about 22° C.) for drying.

The test bacteria Staphylococcus aureus ATCC 6538, Escherichia coli ATCC 10536, Pseudomonas aeruiginosa ATCC 15442 and the yeast Candida albicans ATCC 10231 are grown in liquid nutrient media at 37° C. for 24 hours. The test fungi Aspergillus niger ATCC 6275 is grown in Sabouraud media for 3 days at 28° C. In case of Malassezia furfur DSM 6171, CIP 1636-86 an agarpetridish grown for 8 days at 30° C. is washed with 10 ml sterile water and the suspension is stirred for 30 minutes.

The cell suspensions of the test organisms are diluted in 0.85% sodium chloride as follows:

1:100 dilution of Staphylococcus aureus ATCC 6538

1:1000 dilution of Escherichia coli ATCC 10536

1:1000 dilution of Pseudomonas aeruginosa ATCC 15442

1:10 dilution of Candida albicans ATCC 10231

1:10 dilution of Aspergillus niger ATCC 6275

1:10 dilution of Malassezia furfur DSM 6171, CIP 1636-86

10 μl of these cell dilutions are then dotted onto the surface of the agar media. The so inoculated agar plates are then incubated for 24 hours at 37° C. (for bacteria and the yeast) or 3 days at 28° C. (for Aspergillus niger), 4 days at 30° C. and then the highest dilution of the test compound at which no more growth was observed (corresponds to the MIC) is determined.

Growth controls are conducted for all strains with the pure growth medium (Müller-Hinton for S. aureus, E. coli, Ps. aeruginosa, C. albicans; Sabouraud for A. niger and Medium 472 for M. furfur) as well as with ethanol.

TABLE B
MIC-values [in ppm] conducted to determine the microstatic activities:

| | M. fufur |
|---|---|
| Compound P-5 | 50 |

1. A method for protecting skin, mucosa and integumentary appendages against the action of microbes including protecting the scalp from dandruff, which comprises applying thereto an effective amount of pyranopyranone derivatives of formula (1) 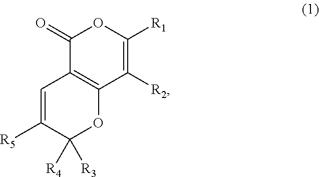
wherein
R₁, R₂, R₃, R₄ and R₅ independently of one another are hydrogen; C₁-C₃₀alkyl, C₂-C₃₀alkenyl, or C₃-C₁₂cycloalkyl, each of which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₇-C₂₅aralkyl, or —CO—R₆; —SiR₈R₉R₁₀; or
R₄ and R₅ together form a five or six membered ring; or
R₃ and R₄ together form a five or six membered ring;
D is —CO—; —COO—; —S—; —SO—; —SO₂—; —O—; —NR₇—; —SiR₈R₉—; —POR₁₀—; —CR₁₂═CR₁₃—; or —C≡C—; and
E is —OR₆; —SR₆; —NR₁₄R₁₅; —NR₁₄COR₁₅; —COR₆; —COOR₆; —CONR₁₄R₁₆; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈perfluoroalkyl; C₁-C₁₈alkoxy, which is optionally substituted by E and/or interrupted by D; wherein
R₆ is H; C₆-C₁₈aryl which is optionally substituted by OH, C₁-C₃₀alkyl or C₁-C₃₀alkoxy; or C₁-C₃₀alkyl which is optionally interrupted by —O—;
R₇ is H; C₆-C₁₈aryl which is optionally substituted by OH, C₁-C₃₀alkyl or C₁-C₃₀alkoxy; or C₁-C₃₀alkyl which is optionally interrupted by —O—;
R₈, R₉ and R₁₀ independently of each other are hydrogen; C₁-C₃₀alkyl; C₆-C₁₈aryl which is optionally substituted by C₁-C₃₀alkyl; and
R₁₁ is hydrogen; C₆-C₁₈aryl, which is optionally substituted by OH, C₁-C₃₀alkyl or C₁-C₃₀alkoxy; or C₁-C₃₀alkyl, which is optionally interrupted by —O—;
R₁₂, R₁₃, R₁₄ and R₁₅ independently of each other are hydrogen; C₆-C₁₈aryl which is optionally substituted by OH, C₁-C₃₀alkyl or C₁-C₃₀alkoxy; C₁-C₃₀alkyl, which is optionally interrupted by —O—; or
R₁₄ and R₁₅together form a five or six membered ring;
as an antimicrobial and/or preserving agent.

2. The method according to claim 1, wherein
R₁ is C₁-C₆alkyl;
R₂ is hydrogen;
R₃, R₄ and R₅ independently of one another are hydrogen; C₁-C₃₀alkyl, C₂-C₃₀alkenyl, or C₃-C₁₂cycloalkyl, which may be substituted by one or more E and/or interrupted by one or more D; C₆-C₂₀aryl, which may be substituted by one or more G; C₄-C₂₀heteroaryl, which may be substituted by one or more G, C₂-C₁₈alkenyl, C₂-C₁₈alkynyl, C₇-C₂₅aralkyl, or —CO—R₆; or
R₄ and R₅ together form a five or six membered ring;
D is —CO—; —COO—; —O—; NR₇—; —CR₁₂═CR₁₃—; or —C≡C—; and
E is —OR₆; —NR₁₄R₁₅; —NR₁₄COR₁₅; —COR₆; —COOR₆; —CONR₁₄R₁₅; —CN; halogen; or OSO₃R₁₁; SO₃R₁₁; SO₂R₁₁; PO₃(R₁₁)₂; OPO₃(R₁₁)₂;
G is E; C₁-C₁₈alkyl, which is optionally interrupted by D; C₁-C₁₈perfluoroalkyl; C₁-C₁₈alkoxy, which is optionally substituted by E and/or interrupted by D; wherein
R₆, R₇, R₁₁, R₁₂, R₁₃, R₁₄ and R₁₅ are defined as in formula (1).

3. The method according to claim 1, wherein
R₁ is methyl;
R₂ is hydrogen;
R₃, R₄ and R₅ independently of one another are hydrogen; or C₁-C₃₀alkyl, C₂-C₃₀alkenyl, phenyl, pyridinyl, pyrimidinyl, triazinyl, pyrrolyl, furanyl, thiophenyl or chinolinyl, each of which is unsubstituted or substituted by G; or Si(C₁-C₄alkyl)₃; and
R₃ and R₄ together, or R₄ and R₅ together, may be C₂-C₈alkylene, C₂-C₈alkenylene, C₂-C₈oxaalkylene, C₂-C₈oxaalkenylene, C₄-C₈cycloalkylene, C₄-C₈cycloalkenylene, each of which is unsubstituted or substituted by G;
G is C₁-C₈alkyl, C₁-C₈alkoxy, OH, where two adjacent residues G may be interconnected to form C₃-C₄alkylene or a residue —O—CH₂—O—.

4. The method according to claim 1, wherein
R₁ is methyl;
R₂ is hydrogen;
R₃, R₄ and R₅ independently of one another are hydrogen; C₁-C₃₀alkyl, or C₂-C₃₀alkenyl, or
R₃ and R₄ together form a five or six membered ring, which may be interrupted by —O—; or
R₄ and R₅ together form a five or six membered ring.

5. The method according to claim 1, wherein
R₁ is methyl
R₂ is hydrogen
R₃ is hydrogen; C₁-C₁₂alkyl; C₂-C₄alkenyl; or a radical of formula 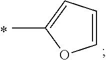
R₄ is hydrogen; C₂-C₄alkenyl;
R₅ is hydrogen; C₁-C₄alkyl; phenyl; —SiR₈R₈R₁₀; or a radical of formula 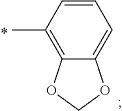
or
R₃ and R₄ together form the rings 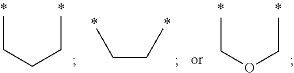
or
R₄ and R₅ together form the rings

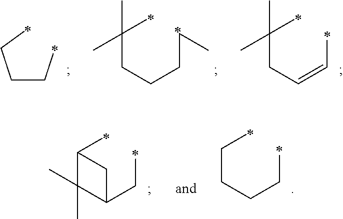

6. A method for protecting paper, wood, leather, synthetic textile materials or natural textile materials against the action of microbes comprising incorporating or applying an effective amount thereto of pyranopyranone derivatives of formula (1) according to claim 1.

7. A method for cleaning and disinfecting hard surfaces which comprises applying a preparation comprising an effective amount thereto of pyranopyranone derivatives of formula (1) according to claim 1 as an antimicrobial and/or preserving agent.

8. A method for preventing bio-fouling of an article comprising incorporating the pyranopyranone derivatives of formula (1) according to claim 1 into the article or surface of the article or by applying the pyranopyranone derivative of formula (1) to these surfaces either directly or as part of a coating or film.

9. Personal care preparations comprising
0.01 to 15 weight % of the pyranopyranone derivative of formula (1) according to claim 1 and a cosmetically compatible carrier and/or adjuvants.

10. The compound of formula [chemical structure]
